(19) United States
(12) Patent Application Publication
(10) Pub. No.: US 2019/0241659 A1
(43) Pub. Date: Aug. 8, 2019

(54) BISPECIFIC FUSION POLYPEPTIDES AND METHODS OF USE THEREOF

(71) Applicant: MEDIMMUNE, LLC, Gaithersburg, MD (US)

(72) Inventors: JOHN MUMM, GAITHERSBURG, MD (US); YUE WANG, GAITHERSBURG, MD (US); RYAN GILBRETH, GAITHERSBURG, MD (US); RONALD HERBST, GAITHERSBURG, MD (US); SCOTT HAMMOND, GAITHERSBURG, MD (US); MICHAEL OBERST, GAITHERSBURG, MD (US); GODFREY JONAH RAINEY, Gaithersburg, MD (US); SIMON DOVEDI, CAMBRIDGE (GB); JONATHAN SEAMAN, CAMBRIDGE (GB); MICHELLE TURNHAM, CAMBRIDGE (GB)

(21) Appl. No.: 16/184,251

(22) Filed: Nov. 8, 2018

Related U.S. Application Data
(60) Provisional application No. 62/583,723, filed on Nov. 9, 2017.

(51) Int. Cl. C07K 16/28; A61K 39/395; A61P 35/00; A61K 45/06

(52) U.S. Cl. CPC C07K 16/2818; C07K 16/2827; A61K 39/39541; A61P 35/00; A61K 45/06; A61K 2039/505; C07K 2317/622; C07K 2317/52; C07K 2317/55; C07K 2317/53; C07K 2317/31

(57) ABSTRACT

Provided herein are bispecific fusion proteins and methods of using the bispecific fusion proteins for treating cancer.

### BACKGROUND OF THE INVENTION

Cancer continues to be a major global health burden. In 2016, in the United States alone, it was estimated that more than 1.5 million new cases would be diagnosed and more than 500,000 people would die from the disease (see Cancer Statistics from the National Cancer Institute, National Institutes of Health). Globally, it is estimated that nearly 1 in 6 deaths can be attributable to cancer (see Cancer Fact Sheet, February 2017, World Health Organization).

Despite the significant progress made over the past decade in developing strategies for combatting cancer and other diseases, patients with advanced, refractory and metastatic disease have limited clinical options. Chemotherapy, irradiation, and high dose chemotherapy have become dose limiting (and in many cases merely extend patient life albeit with significantly debilitating side-effects). Therefore, there continues to be an unmet medical need for new, effective anticancer therapies for those patients with advanced and/or treatment-resistant cancers. Moreover, there is also a need for less toxic, more targeted anticancer therapies.

A potential source for new, non-toxic anticancer therapies is a patient's own immune system. The role of the immune system, in particular T cell-mediated cytotoxicity, in tumor control is well recognized. There is mounting evidence that T cells can control tumor growth and survival in cancer patients, both in early and late stages of the disease. However, tumor-specific T-cell responses are difficult to mount and sustain in cancer patients.

Examples of T cell signaling pathways that can influence tumor-specific T cell responses involve signaling proteins such as cytotoxic T lymphocyte antigen-4 (CTLA-4, CD152), programmed death ligand 1 (PD-L1, also known as B7-H1 or CD274), CD40 ligand (CD40L), glucocorticoid-induced TNF receptor (TNFR)-related protein (GITR), OX40, and CD137 (4-1BB).

CD40L is a member of the tumor necrosis factor (TNF) family of molecules which is primarily expressed on activated T cells (including Th0, Th1, and Th2 subtypes), and forms homotrimers similar to other members of this family. Further, CD40L has also been found expressed on mast cells, and activated basophils and eosinophils. CD40L binds to its receptor CD40 on antigen-presenting cells (APC), which leads to many effects depending on the target cell type. In general, CD40L plays the role of a costimulatory molecule and induces activation in APC in association with T cell receptor stimulation by MHC molecules on the APC.

Signaling through CD40 by CD40L initiates a cascade of events that results in the activation of the CD40-bearing cells and optimal T cell priming. More specifically, CD40L/CD40 signaling promotes differentiation of B cells into antibody secreting and memory B cells (Burkly, In Adv. Exp. Med. Bio., Vol. 489., D.M. Monroe, U. Hedner, M.R. Hoffman, C. Negrier, G.F. Savidge, and G.C.I. White, eds. Klower Academic/Plenum Publishers, 2001, p. 135). Additionally, CD40L/CD40 signaling promotes cell-mediated immunity through activation of macrophages and dendritic cells, which promote anti-tumor immune responses through natural killer cells and the stimulation of tumor antigen specific cytotoxic T lymphocytes (see Burkly, supra).

PD-L1 is also part of a complex system of receptors and ligands involved in controlling T cell activation. In normal tissue, PD-L1 is expressed on T cells, B cells, dendritic cells, macrophages, mesenchymal stem cells, bone marrow-derived mast cells, and various non-hematopoietic cells. Its normal function is to regulate the balance between T-cell activation and tolerance through interaction with its two receptors: programmed death 1 (also known as PD-1 or CD279) and CD80 (also known as B7-1 or B7.1). PD-L1 is also expressed in a broad range of cancers with a high frequency and acts at multiple sites to help tumors evade detection and elimination by the host immune system. In some cancers, expression of PD-L1 has been associated with reduced survival and unfavorable prognosis. Antibodies that block the interaction between PD-L1 and its receptors (e.g., PD-1) are able to relieve PD-L1 -dependent immunosuppressive effects and enhance the cytotoxic activity of antitumor T cells in vitro and in vivo.

GITR (also known as TNFRSF18, AITR or CD357) is expressed on regulatory T cells and is up-regulated on antigen experienced CD4⁺ helper cells and CD8⁺ cytotoxic T cells as well as activated natural killer cells (Stephens et al. J. Immunol. (2004) 173(8): 5008-5020; Clothier and Watts, Cytokine Growth Factor Rev. (2014)). GITR is part of a complex system of receptors and ligands that are involved in controlling T cell activation by antigen exposure. GITR has one known endogenous ligand, GITR ligand (GITRL), that exists in a loosely trimeric form and can cluster GITR resulting in potent cell signaling events within T cells (Chattopadhyay et al. (2007) Proc. Natl. Acad Sci. USA 104(49): 19452-19457). The interaction between GITR and GITRL delivers a positive costimulatory signal to T cells, which enhances their proliferation and activation by antigen exposure, helps to promote memory cell generation, and reprograms regulatory T cells to reduce their suppressive functions (Clothier and Watts, Cytokine Growth Factor Rev. (2014) January 4; Schaer et al. Curr Opin Immunol. (2012)).

OX40 (CD134; TNFRSF4) is another TNF receptor found primarily on activated CD4⁺ and CD8⁺ T-cells, regulatory T cells (Treg) and natural killer cells (Croft et al., 2009, Immunol Rev. 229:173-91). OX40 has one known endogenous ligand, OX40 ligand (OX40L; CD152; TNFSF4), that exists in a trimeric form and can cluster OX40 resulting in potent cell signaling events within T cells (Croft et al., 2009, Immunol Rev. 229:173-91). Signaling through OX40 on activated CD4⁺ and CD8⁺ T cells leads to enhanced cytokine production, granzyme and perforin release, and expansion of effector and memory T cell pools (Jensen et al., 2010, Semin Oncol. 37:524-32). In addition, OX40 signaling on Treg cells inhibits expansion of Tregs, shuts down the induction of Tregs and blocks Treg-suppressive function (Voo et al., 2013, J Immunol. 191:3641-50; Vu et al., 2007, Blood. 110:2501-10).

Immunohistochemistry studies and early flow cytometry analyses showed that OX40 is expressed on T cells infiltrating a broad range of human cancers (Baruah et al., 2011, Immunobiology 217:668-675; Curti et al, 2013, Cancer Res. 73:7189-98; Ladanyi et al, 2004, Clin Cancer Res. 10:521-30; Petty et al, 2002, Am J Surg. 183:512-8; Ramstad et al, 2000, Am J Surg. 179:400-6; Sarff et al, 2008, Am J Surg. 195:621-5; discussion 625; Vetto et al, 1997, Am J Surg. 174:258-65). OX40 expression on tumor-infiltrating lymphocytes correlates with longer survival in several human cancers, suggesting that OX40 signals may play a critical role in establishing an antitumor immune response (Ladanyi et al., 2004, Clin Cancer Res. 10:521-30; Petty et al., 2002, Am J Surg. 183:512-8).

CD137 (4-1BB), like GITR, is a costimulatory checkpoint molecule that is expressed on activated T cells and NK cells. CD137L (CD137 ligand) is expressed by antigen presenting cells and has been associated with enabling the immune system to eliminate tumors in multiple cancer types. CD137 is expressed at higher levels on CD8⁺ than CD4⁺ T cells, and it mainly co-stimulates CD8⁺ T cells. Crosslinking of CD137 strongly enhances proliferation, IFN-γ secretion and cytolytic activity of T cells. Moreover, CD137 agonists, such as antibodies, have been reported to work synergistically with cancer vaccines and immune check point inhibitors to boost anticancer immune responses. (Dharmadhikari et al., 2016, Oncoimmunology 5(4): e1113367).

The aforementioned T cell signaling pathways (and others) each plays a role in controlling tumor-specific T cell responses. However, the relative importance of different T cell signaling pathways in the context of inducing and maintaining a desired T cell-mediated antitumor response remains to be elucidated. Indeed, interplay between different T cell signaling pathways may lead to synergistic effects in the context of treating cancer. Therefore, there is a need in the art for novel agents capable of maximizing T cell-mediated cytotoxicity via improved control of T cell signaling pathways. Such agents could provide less toxic, more targeted anticancer therapies.

### SUMMARY OF THE INVENTION

Provided herein are bispecific fusion proteins and methods of their use for controlling T cell-mediated cytotoxicity.

In one aspect, the disclosure herein provides a bispecific fusion protein, comprising a single chain fusion protein comprising a first binding region specific for a first cell surface target, an Fc monomer, and a second binding region specific for a second cell surface target, wherein the first binding region and the second binding region are covalently linked to the Fc monomer via a peptide linker, and wherein the bispecific fusion protein is capable of binding the first cell surface target and the second cell surface target at the same time.

In certain aspects, the at least one of the first binding region and the second binding region is a Fab fragment or a receptor ligand.

In further aspects, the Fab fragment is an anti-PD-1 or anti-PD-L1 Fab fragment.

In additional aspects, the at least one of the one or more ligand subunits is GITRL, OX40L, TNF-α. CD137L or CD40L.

In another aspect, the disclosure herein provides a method of treating cancer comprising treating a patient in need thereof with the bispecific fusion protein as disclosed herein.

These and other features and advantages of the present disclosure will be more fully understood from the following detailed description of the disclosure taken together with the accompanying claims. It is noted that the scope of the claims is defined by the recitations therein and not by the specific discussion of features and advantages set forth in the present description.

### BRIEF DESCRIPTION OF THE DRAWINGS

FIG. 1. Schematic representation of a contemplated bispecific fusion protein (BFP) including a first binding domain (BD1), a second binding domain (BD2), and an immunoglobulin Fc region. BD2 can be attached to the Fc region via a hinge region portion of the Fc region. BD1 can be attached to the Fc region via peptide linkers. Similarly, subunits of the BD1 portion (here shown with 3 subunits, though fewer or more are contemplated) can be interconnected via peptide linkers.

FIG. 2. Different structures of contemplated fusion proteins including monospecific and bispecific fusion proteins. One monospecific fusion protein is MEDI5083, which has a BD1_Fc region format akin to an IgG antibody and that is produced as a dimerized single chain fusion protein, where BD1=2×CD40L trimer, with the CD40L subunits connected by peptide linkers, and Fc is a single IgG4 Fc region (CH2 and CH3)). MEDI5083 is structurally comparable to MEDI4736 (Durvalumab, an anti-PD-L1 antibody, where BD1=anti-PD-L1 F(ab)2; Fc=IgG1 TM (“triple mutation,” with L234F/L235E/P331S in human IgG1). In contrast are two iterations (BFP2 and BFP3) of bispecific fusion protein formats. BFP2, which is 203 kDa in size in the iteration shown, has a BD1_Fc_BD2 format (N to C), where BD1 is 2×CD40L trimers, Fc is an IgG4 Fc region, and BD2 is 2×anti-PD-L1 scFv. BFP3, which is 242 kDa in size in the iteration shown, has a BD2_Fc_BD1 format, where BD2 is an anti-PD-L1 F(ab)2 (e.g., taken from MEDI4736), Fc is a single IgG4 Fc region, and BD1 is 2×CD40L trimer.

FIGS. 3A-3T. Specific embodiments of contemplated BFP3s. FIG. 3A depicts a bispecific fusion protein including 2 Fab fragments targeting PD-1, an IgG1 Fc polypeptide core, and 6 GITRL subunits. FIG. 3B depicts a bispecific fusion protein including 2 Fab fragments targeting PD-L1, an IgG1 Fc polypeptide core, and 6 GITRL subunits. FIG. 3C depicts a bispecific fusion protein including 2 Fab fragments targeting PD-1, an IgG1 Fc polypeptide core, and 6 OX40L subunits. FIG. 3D depicts a bispecific fusion protein including 2 Fab fragments targeting PD-L1, an IgG1 Fc polypeptide core, and 6 OX40L subunits. FIG. 3E depicts a bispecific fusion protein including 2 Fab fragments targeting PD-1, an IgG1 Fc polypeptide core, and 6 CD40L subunits. FIG. 3F depicts a bispecific fusion protein including 2 Fab fragments targeting PD-L1, an IgG1 Fc polypeptide core, and 6 CD40L subunits. FIG. 3G depicts a bispecific fusion protein including 2 Fab fragments targeting PD-1, an IgG1 Fc polypeptide core, and 6 TNF-α subunits. FIG. 3H depicts a bispecific fusion protein including 2 Fab fragments targeting PD-L1, an IgG1 Fc polypeptide core, and 6 TNF-α subunits. FIG. 3I depicts a bispecific fusion protein including 2 Fab fragments targeting PD-1, an IgG1 Fc polypeptide core, and 6 CD137L subunits. FIG. 3J depicts a bispecific fusion protein including 2 Fab fragments targeting PD-L1, an IgG1 Fc polypeptide core, and 6 CD137L subunits. FIG. 3K depicts a bispecific fusion protein including 2 Fab fragments targeting PD-1, an IgG4 Fc polypeptide core, and 6 GITRL subunits. FIG. 3L depicts a bispecific fusion protein including 2 Fab fragments targeting PD-L1, an IgG4 Fc polypeptide core, and 6 GITRL subunits. FIG. 3M depicts a bispecific fusion protein including 2 Fab fragments targeting PD-1, an IgG4 Fc polypeptide core, and 6 OX40L subunits. FIG. 3N depicts a bispecific fusion protein including 2 Fab fragments targeting PD-L1, an IgG4 Fc polypeptide core, and 6 OX40L subunits. FIG. 30 depicts a bispecific fusion protein including 2 Fab fragments targeting PD-1, an IgG4 Fc polypeptide core, and 6 CD40L subunits. FIG. 3P depicts a bispecific fusion protein including 2 Fab fragments targeting PD-L1, an IgG4 Fc polypeptide core, and 6 CD40L subunits. FIG. 3Q depicts a bispecific fusion protein including 2 Fab fragments targeting PD-1, an IgG4 Fc polypeptide core, and 6 TNF-α subunits. FIG. 3R depicts a bispecific fusion protein including 2 Fab fragments targeting PD-L1, an IgG4 Fc polypeptide core, and 6 TNF-α subunits. FIG. 3S depicts a bispecific fusion protein including 2 Fab fragments targeting PD-1, an IgG4 Fc polypeptide core, and 6 CD137L subunits. FIG. 3T depicts a bispecific fusion protein including 2 Fab fragments targeting PD-L1, an IgG4 Fc polypeptide core, and 6 CD137L subunits.

FIGS. 4A-4C. Additional specific BFP3 formatted bispecific fusion proteins: anti-PD-L1_Fc_TNF-α (FIG. A), anti-PD1_Fc_OX40L (FIG. B); and anti-PD1_Fc_GITRL (FIG. C). The anti-PD-L1 F(ab) fragment was derived from MEDI4736, and the anti-PD-1 F(ab) fragments were derived from an anti-PD1 antibody (LO115). The TNF-α, OX40L, and GITRL binding domains each include 2 sets of trimer repeats of each protein subunit linked together via peptide linkers.

FIGS. 5A-5D. BFP 2 and 3 bind with CD40 and PD-L1. FIGS. 5A-5D demonstrate the concurrent binding of CD40 and PD-L1 proteins by anti-PD-L1_IgG4 Fc_CD40L BFP 2 and 3 molecules by an Octet assay.

FIGS. 6A and 6B. BFP2 & BFP3 retain the ability to bind CD40. Anti-PD-L1_IgG4 Fc_CD40L BFP 2 and 3 molecules demonstrate similar abilities to bind to cell surface CD40 compared to the parental CD40L FP (fusion protein) in a flow cytometry based assay.

FIGS. 7A and 7B. BFP2 & BFP3 Have Lower Binding to PDL1 on Cell Surface. FIGS. 7A and 7B demonstrates that anti-PD-L1_IgG4 Fc_CD40L FP BFP 2 and 3 molecules can bind to cell surface PD-L1 protein in a flow cytometry based assay.

FIG. 8A and 8B. BFP Binding to Mixed PBMCs. FIGS. 8A and 8B demonstrates that anti-PD-L1_IgG4 Fc_CD40L FP BFP 2 and 3 molecules bind to human PBMC subsets in a dose-dependent manner, similar to the parental anti-PD-L1 and CD40L FP in a flow cytometry-based assay.

FIG. 9. BFP2 & BFP3 Retain the Capacity to Stimulate CD40 Signaling Pathway. FIG. 9 demonstrates that anti-PD-L1_IgG4 Fc_CD40L FP BFP 2 and 3 molecules activate the NF-κB signaling pathway, which is downstream of CD40 activation on multiple cell types.

FIG. 10. BFP3 block PD-L1 and PD1 interaction and enhance NFAT signaling in Jurkat T cells. FIG. 10 demonstrates that the anti-PD-L1_IgG4 Fc_CD40L FP BFP 2 and 3 molecules block PD-L1-PD1 interaction, resulting in activation of NFAT pathway in Jurkat cells.

FIGS. 11A and 11B. BFPs Stimulate CD40 and Block PD1-PDL1 Interaction. FIG. 11A illustrates a co-stimulatory assay conceptual schematic. FIG. 11B shows that anti-PD-L1-CD40L BFP has dual functions: it activated NF-κB on THP-1 cells through CD40 engagement and enhanced NFAT activity in Jurkat cells by removing PD-L1-mediated inhibition.

FIG. 12. BFP3 has superior IL-2 inducing activity in SEB Assay. FIG. 12 shows results from a Staphylococcal enterotoxin B (SEB) assay, demonstrating that anti-PD-L1_IgG4 Fc_CD40L FP BFP 2 and 3 molecules induce more IL-2 production than the combination of parental molecules CD40L FP and anti-PD-L1.

FIG. 13. PD-1GITRL Bispecifics (MEDI3387 and MEDI5771) Increase T-cell Activation Versus Single Agents in the SEB Assay. FIG. 13 demonstrates that MEDI3387 and MEDI5771 had activity equivalent to the combination of parent molecules but greater than either parent molecule alone. The results were comparable across 2 batches and demonstrated similar results to the T cell reactivation assay.

FIG. 14. BFP3 Induces Robust IFN-γ and IL-12 Production in M1 Macrophage-T MLR Assay. FIG. 14 shows results from a macrophage-T cell MLR assay, demonstrating that anti-PD-L1_IgG4 Fc_CD40L FP BFP 2 and 3 molecules induce more IFN-γ and IL-12 production than the combination of parental molecules CD40L FP and anti-PD-L1.

FIG. 15. BFP3 Induces Robust IFN-γ Production in Mono-T MLR Assay. FIG. 15 shows results from a monocyte-T cell MLR assay, demonstrating that anti-PD-L1_IgG4 Fc_CD40L FP BFP 2 and 3 molecules induce more or equivalent amounts of IFN-γ production than the combination of parental molecules CD40L FP and anti-PD-L1.

FIG. 16. BFP3 shows superior activity over combo in CMV recall assay. FIG. 16 shows results from a CMV antigen recall assay, demonstrating that anti-PD-L1_IgG4 Fc_CD40L FP BFP3 molecules induce more IFN-γ, IL-12, and IL-10 production but similar levels of TNF-α, IL-1β, IL-6 and IL-8 compared to the combination of parental molecules CD40L FP and anti-PD-L1.

FIGS. 17A-17B. BFP3 can potentially alter membrane localization of CD40 and PD-L1. FIG. 17A illustrates that CD40 and PD-L1 co-express on antigen presenting cells (APC). FIG. 17B shows a conceptual schematic for a flow cytometry-based assay for studying cell surface CD40 and PD-L1 proteins.

FIG. 18. BFP3 induces down-regulation of CD40 and PD-L1 on MDA-MB-231 cells. FIG. 18 shows results from a flow cytometry-based assay, demonstrating that only anti-PD-L1_IgG4 Fc_CD40L FP BFP3 molecules can induce down-regulation of both CD40 and PD-L1 molecules at 1 and 96 hours post-treatment.

FIG. 19. BFP3 induces down-regulation of CD40 and PD-L1 on MDA-MB-231 cells. FIG. 19 shows results from a Western blot, demonstrating that anti-PD-L1_IgG4 Fc_CD40L FP BFP3 molecules induce down-regulation of total PD-L1 protein content at 24 hours post-treatment. NS=no stimulation.

FIG. 20. Loss of surface CD40 & PD-L1 in THP1 cells upon BFP3 stimulation. FIG. 20 shows a conceptual schematic for an assay for studying cell surface CD40 and PD-L1 proteins, where continual stimulation is employed.

FIG. 21. Loss of surface CD40 & PD-L1 in THP1 cells upon BFP3 stimulation. FIG. 21 shows results from a flow cytometry-based assay, demonstrating that anti-PD-L1_IgG4 Fc_CD40L FP BFP3 molecules induce down-regulation of surface CD40 and PD-L1 at 0.5 to 3 hours post-treatment.

FIG. 22. Loss of surface PD-L1 in THP1 cells upon BFP3 stimulation. FIG. 22 shows a conceptual schematic for an assay for studying cell surface CD40 and PD-L1 proteins after 1 hour treatment, followed by washing off testing materials.

FIG. 23. Loss of surface PD-L1 in THP1 cells upon BFP3 stimulation. FIG. 23 shows results from a flow cytometry-based assay on THP-1 cells, demonstrating that transient treatment with anti-PD-L1_IgG4 Fc_CD40L FP BFP3 molecules for one hour induces down-regulation of cell surface CD40 and PD-L1 proteins. At 24 hours, only CD40 can be detected on the cell surface.

FIG. 24. BFP3 treated moDC have lower amount of PD-L1 protein. FIG. 24 shows results from a flow cytometry-based assay, demonstrating differentiated effects between of anti-PD-L1_IgG4 Fc_CD40L FP BFP3 and CD40L FP molecules in regulating cell surface expression of CD40, CD86, and PD-L1: down-regulation of PD-L1 was achieved only in BFP3 treated cells.

FIG. 25. BFP3 treated moDC have lower amount of PD-L1 protein. FIG. 25 shows results from a Western blot, demonstrating that amounts of PD-L1 protein are much less in anti-PD-L1_IgG4 Fc_CD40L FP BFP3 treated conditions than with treatment of CD40L FP alone or CD40L FP plus anti-PD-L1.

FIG. 26. Loss of surface CD40 and PD-L1 on Blood Monocytes. FIG. 26 shows results from a flow cytometry based assay, demonstrating differentiated effects between of anti-PD-L1_IgG4 Fc_CD40L FP BFP3 and CD40L FP molecules in regulating cell surface expression of CD40 and PD-L1: dose-dependent down-regulation of PD-L1 was achieved only in BFP3 treated cells.

FIG. 27. mBFP3 induces degradation of murine PD-L1 in Renca cells. FIG. 27 shows results from Western blot, demonstrating that the amount of murine PD-L1 protein is much less in anti-PD-L1_IgG4 Fc_CD40L FP BFP3 treated conditions than the treatment of CD40L FP alone or CD40L FP plus anti-PD-L1.

FIG. 28. PD-L1 Cross-linking Increases NF-κB activity Mediated by BFP3. FIG. 28 shows that cross-linking PD-L1 on the surface of ES2 cells can enhance NF-κB action on THP-1 cells mediated by BFP3. Y-axes show NF-x3 reporter activity.

FIG. 29. IgG4 Fc and FcγRI Interaction Modulate BFP3 Activities. FIG. 29 shows that FcγR can augment NF-x3 action on THP-1 cells mediated through BFP3.

FIGS. 30A-30F. Weight-loss is less in mBFP3 treated mice (single dose treatment). FIGS. 30A-30F show results from multiple studies on wild type mice and mice implanted with B16F10 tumor cells. Changes of body weight post treatment from individual mice are shown.

FIG. 31. Weight-loss is less in mBFP3 treated mice (multiple dose treatment). FIG. 31 shows results from multiple dose studies on mice implanted with B16F10 tumor cells. Percent changes in body weight post treatment from individual mice are shown.

FIG. 32. mBFP3 treatment effectively inhibits tumor growth. FIG. 32 shows results from multiple dose studies (twice per week×2weeks) on mice implanted with B16F10 tumor cells. Changes of tumor volume post treatment from individual mice are shown.

FIGS. 33A-33C. Reducing dosing frequency of BFP3 prevents side effects. FIGS. 33A-33C show results from reduced dose studies (one or twice dosing) on mice implanted with B16F10 tumor cells. Changes of body weight post-treatment from individual mouse were presented in FIG. 33A. FIG. 33B shows changes of tumor volume, and FIG. 33C shows levels of serum Alanine transaminase (ALT).

FIG. 34. mBFP3 treatment induced activation/differentiation of T cells. FIG. 34 shows results from a flow cytometry study on T cells recovered from B16F10 tumor bearing mice. Percentages of T cell subsets were determined and shown in graphs.

FIG. 35. mBFP3 treatment induces effector/memory CD8 T cell differentiation. FIG. 35 shows results from flow cytometry assay studying T cells recovered from B16F10 tumor bearing mice. Percentages of effector CD8 T cell subset were determined and shown in graphs.

FIG. 36. MEDI7526 in mice does not induce TNF-α or IL-6, two key mediators of immune-related toxicities. FIG. 36 shows that neither TNF-α nor IL-6 appears to be required for MEDI7526's anti-tumor function.

FIG. 37. MEDI7526 mouse surrogate induces distinct cytokine profile in mice (single iv dosing study).

FIG. 38. PD1-OX40L induces NF-κB activation in Jurkat/OX40 cells. FIG. 38 demonstrates that the anti-PD-L_IgG4 Fc_OX40L FP BFP3 molecule activates the NF-κB signaling pathway in a Jurkat cell line transfected with OX40.

FIG. 39. Effect of mouse OX40 ligand (mOX40L) fusion protein (FP), anti-mouse PD-L1monoclonal antibodies (mAb) or the combination of mOX40L FP and anti-PD-L1 mAb on the growth of MCA205 and CT26 cell lines in mouse syngeneic models.

FIGS. 40A-40C. PD-L1-dependent tumor localization of PDL1OX40L FP BFP2 (MEDI5615).

FIG. 41. Biodistribution in tumor-bearing mice of different molecular formats of PD-L1/OX40L bispecific molecules.

FIG. 42. Cell systems used for measuring bioactivity of bispecific molecules.

FIG. 43. BFP2 has optimal valence for PD-L1-mediated clustering of OX40. RLU=relative light units; M=Molarity.

FIGS. 44. MEDI5615 (PDL1/OX40L BFP2) and scOX40L increased T-effector proliferation in the presence of natural CD4+CD25+Treg cells and decreased the frequency of IL-10 producing T regulatory cells. Error bars represent the standard error of the mean from duplicate assay wells.

FIGS. 45A-45B. Shows activity of PDL1OX40L FP BFP2 (MEDI5615) and OX40/PDL1 bispecific mAbs in a staphylococcal enterotoxin B (SEB) co-stimulation assay.

FIGS. 46A-46F. Shows binding of PD-L1OX40L BFP2 to CHO cells engineered to express human or cynomolgus monkey OX40, PD-L1, or both OX40 and PD-L1. Error bars represent standard deviation of the mean. MFI=mean fluorescence intensity.

FIG. 47. PD1-OX40L triggers degradation of PD1 in human PBMC. FIG. 47 shows results from a Western blot, demonstrating that PD-1 protein levels are reduced in anti-PD-1_IgG4 Fc_OX40L FP BFP3 treated conditions but OX40 protein levels were not changed.

FIG. 48. MEDI3387 triggers degradation of PD1 in human PBMC. FIG. 48 shows results from a Western blot, demonstrating that PD-1 protein levels are reduced in anti-PD-1_IgG4 Fc_GITRL FP BFP3 treated conditions but the amount of GITR protein was not changed. PD-1/GITRL FP Bis; MEDI5771 (IgG1 format) & MEDI3387 (IgG4P); GITRL: MEDI1873.

FIG. 49. MEDI3387 induces NF-κB activation in Jurkat/GITR cells. Four hour stimulation. FIG. 49 demonstrates that the anti-PD-L_IgG4 Fc_GITRL FP BFP3 molecule activates the NF-κB signaling pathway in a Jurkat cell line transfected with GITR.

FIG. 50. PD1/GITR Bispecific Molecules Can Bind Simultaneously to Both Targets. FIG. 50 demonstrates the concurrent binding of PD1 and GITR proteins by anti-PD1_IgG4 Fc_GITRL FP BFP2 (MEDI3387) and anti-PD1_IgG1 Fc_GITRL FP BFP2 (MEDI5771) molecules by Octet assay. “A”=MEDI3387; “B”=BFP2-PD1(0075)-GITRL(sc)-G4P; “C”=BFP2-GITRL(sc)-G4P.

FIG. 51. BFP3 block PD-L1 and PD1 interaction and enhance NFAT signaling in Jurkat T cells. FIG. 51 demonstrates that the MEDI3387 (BIOAE003) and MEDI5771 (BIOAE005) molecules block PD-L1-PD1 interaction, resulting in activation of NFAT pathway in Jurkat cells. BIOAE003 & BIOAE005 demonstrate comparable potency to MEDI1873 (GITRL) and parental anti-PD1 IgG.

FIG. 52. PD1/GITR Bispecific Molecules are Equivalent to Combination of GITRL-FP and PD-1 mAb in B16 mouse model.

FIGS. 53A-53B. Dose-dependent increase in CD4+ and CD8+ total memory T cells (Ki67) upon treatment with MEDI3387 and MEDI5771. FIGS. 53A-B show results of pharmacokinetics (PK) and pharmacodynamics (PD) studies in cynomolgus monkeys treated with varied doses of MEDI3387 and MEDI5771.

FIG. 54. Mean Serum MEDI3387 and MEDI5771 Concentration-Time Profiles in Male Cynomolgus Monkeys after Single Intravenous Injection. IV=intravenous; Error bars represent standard deviation of the mean; LLOQ (black dashed line)=lower limit of quantitation. Data below LLOQ (0.050 mg/L; as shown by black dashed line) are plotted at half of LLOQ for illustrative purposes only.

FIGS. 55A-55E. PD1/GITR Bispecific Molecules Can Bind Simultaneously to Both Targets. FIG. 55A demonstrates an assay schematic for the Cytostim T cell Reactivation Assay. FIG. 55B demonstrates results with PD1/GITR IgG4P BFPs (MEDI3387) and parental molecules. FIG. 55C demonstrates results with PD1/GITR IgG4P BFPs (MEDI5771) and parental molecules.

FIG. 56. Shows fluorescence Biodistribution of GITRL in vivo.

FIGS. 57A-57B. Anti-PDL1-TNFα induces down-regulation of murine PD-L1 in T24 tumor cells; APC—antigen per cell. FIG. 57A shows results from a flow cytometry based assay, demonstrating anti-PD-L1_IgG4 Fc_TNFα FP BFP3 treatment downregulates PD-L1 on T24 tumor cells. FIG. 57B shows anti-PD-L1_IgG4 Fc_TNFα FP BFP3 treatment did not affect cell viability.

FIG. 58. Anti-PDL1-TNF-α BFP can stimulate THP1 myeloid cells. FIG. 58 demonstrates that the anti-PD-L1_IgG4 Fc_TNF-α FP BFP3 molecule activates the NF-κB signaling pathway, which is downstream of TNF-α receptor activation.

FIG. 59. BFP3 drives the concomitant internalization of both CD40 and PD-L1 that does not occur with the combination of parental reagents. FIG. 59 is a conceptual schematic depicting a BFP molecule including anti-PD-L1 and CD40L binding domains and that shows the BFP molecule can internalize two targets (CD40 and PD-L1) on the cell surface resulting in degradation of PD-L1 protein. CD40 is resistant to degradation and expression is subsequently recovered at the cell surface.

FIG. 60. Reporter assay scheme. Panel A shows a model disease state where no signal (no assay response) occurs as a result of anti-CD3/anti-CD28 activation from an antigen presenting cell due to inhibition by PD-1/PD-L1 complex formation. In contrast, Panel B depicts successful blockage of PD-1/PD-L1 complex formation via an anti-PD-1 antibody, which leads to reporter molecule (luciferase) expression upon anti-CD3/anti-CD28 activation.

FIG. 61. Shows the results of an SEB assay comparing anti-PD-L1/CD40L FP BFP3 in IgG1 TM and IgG4 formats.

FIG. 62. Shows the results of a CMV recall assay comparing anti-PD-L1/CD40L FP BFP3 in IgG1 TM and IgG4 formats.

FIG. 63. Shows that Anti-PD-1-anti-OX40 Bis2 does not induce NfkB activation in Jurkat cells, whereas anti-PD-1-OX40L FP BFP3 does.

FIG. 64. Shows that Bis2 construct triggers PD1 degradation, indicating degradation of PD1 is independent of OX40 agonist function.

FIG. 65. Combined treatment of 5FU and MEDI7526 (Anti-PDL1-CD40L BFP3) effectively inhibits tumor growth. FIG. 65 shows results from an animal study, in which sequential treatment of 5FU (Day 11) and MEDI7526 (Day 14, 21 and 28) at multiple doses were administrated to mice bearing CT26 tumor cells. Changes of tumor volume post treatment from individual mice are shown. Treatment of 5FU plus MEDI7526 enhances anti-tumor responses mediated by MEDI7526 treatment alone.

FIG. 66. The liver and spleen are the target organs of murine surrogate of MEDI7526. FIG. 66A shows that murine surrogates of MEDI5083 and MEDI7526 accumulated in liver and spleens. FIG. 66B shows human Kupffer cells (residential macrophage in liver) express both CD40 and PD-L1, indicating MEDI7526 can target Kupffer cells in the liver.

FIGS. 67A-67B. MEDI7526 effectively inhibits liver tumor growth. FIG. 67A shows the design of a liver tumor model, in which CT26-luciferase tumor cells are implanted directly into liver. On day 21, livers were recovered post necropsy and tumor burden in the liver were quantified by imaging luciferase activity. FIG. 67B shows tumor burdens (indicated as luminance units) in the liver from MEDI7526 treated mice were significantly lower comparing to those in the isotype control treated animal.

FIGS. 68A-68B. MEDI7526 treatment induces T cell expansion and activation in the liver from the CT26 liver tumor model study. FIG. 68A shows that mice received MEDI7526 had increased numbers of CD8 T cells in the liver comparing to the control mice. FIG. 68B shows that tumor antigen specific CD8 T cells isolated from MEDI7526 treated animal had higher percentages of activated subtype.

FIGS. 69A-69B. Treatment of MEDI7526 is more tolerable than the combination treatment of MEDI5083 and anti-PDL1 in the CT26 liver tumor model. FIG. 69A shows that weight losses were less in MEDI7526 treated mice comparing to MEDI5083 or MEDI5083 plus anti-PDL1 treatment. Changes of body weight post single dose treatment from individual mice are shown. FIG. 69B shows that mortality was relatively higher in mMEDI5083 plus anti-murine PDL1 group.

FIGS. 70A-70C. Additional specific BFP3 formatted bispecific fusion proteins: anti-PD1-Fc-OX40L wild type (FIG. 70A), anti-PD1-Fc-OX40L 2WT (FIG. 70B); and anti-PD1-Fc-OX4OL 1WT (FIG. 70C). The anti-PD-1 F(ab) fragment was derived from an anti-PD1 antibody (L0115). The OX40L part either has conserved wild type sequence (A) or has mutations on residual F180 (B and C).

FIGS. 71A-71C. Anti-PD1-Fc-OX40L 2WT and 1WT have diminished OX40 agonist function on human T cell line. FIGS. 71A and B show that anti-PD1-Fc-OX40L 2WT has slightly reduced binding and internalization comparing to anti-PD1-Fc-OX40L. FIG. 71C shows that anti-PD1-Fc-OX40L 2WT and 1WT had reduced capability of activating NFκB pathway.

FIG. 72A and 72B. Anti-PD1-Fc-OX40L 2WT and 1WT have similar binding and internalization comparing to anti-PD1-OX40L on primary human T cells. Data were generated on purified CD4 and CD8 T cells from two healthy donors.

FIG. 73A and 73B. Anti-PD1-Fc-OX40L 2WT has diminished OX40 agonist functions on human primary T cells. PD1-OX40L and PD1-OX40L 2WT were compared using human T cell stimulation and CMV recall assays. PD1-OX40L 2WT induced much less inflammatory cytokines comparing to PD1-OX40L, which does not carry any mutations on OX40L.

FIGS. 74A and 74B. Anti-PD1-Fc-OX40L 2WT and 1WT induced PD1 degradation on human T cells. Activated human T cells express PD1 protein and the amount of total PD1 protein were significantly reduced post treatment of anti-PD1-OX40L, anti-PD1-OX40L 1WT or anti-PD1-OX40L 2WT. FIG. 74A. shows a representative Western blot picture and FIG. 74B shows pooled results from three donors.

### DETAILED DESCRIPTION OF THE INVENTION

### Definitions

Unless defined otherwise, all technical and scientific terms used herein have the meanings commonly understood by a person skilled in the art to which this disclosure belongs. The following references provide one of skill with a general definition of many of the terms used in this disclosure: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

The term “T cell-mediated cytotoxicity” refers to the targeted killing of cells by cytotoxic T lymphocytes, such as infected cells or cancerous cells.

The term “anti-tumor activity” is meant to refer to any biological activity that reduces or prevents increase in the proliferation or survival of a tumor cell. In one embodiment, the anti-tumor activity is an anti-tumor immune response.

The term “immunomodulatory agent” refers to an agent that enhances an immune response (e.g., anti-tumor immune response). Exemplary immunomodulatory agents of the disclosure include antibodies, an anti-PD-L1 antibody, and fragments thereof, as well as proteins, such as fusion proteins, bispecific fusion proteins, and/or fragments thereof.

The term “CD40L polypeptide” is meant to indicate a polypeptide or fragment thereof having at least about 85% amino acid identity to NCBI Accession No. NP_000065 and having CD40 binding activity. The term “CD40L” refers both to the full length CD40L and to soluble fragments, e.g., extracellular domain forms of CD40L resulting from proteolysis, and to monomeric forms of CD40L as well as oligomeric forms, e.g., trimeric CD40L. Amino acid sequences of membrane-bound and soluble forms of human CD40L are shown below.

By “CD40 polypeptide” is meant a polypeptide or fragment thereof having at least about 85% amino acid identity to NCBI Accession No. NP_001241 and having CD40L binding activity. An exemplary CD40 amino acid sequence is provided below (SEQ ID NO: 22).

By “PD-L1 polypeptide” is meant a polypeptide or fragment thereof having at least about 85% amino acid identity to NCBI Accession No. NP_001254635 and having PD-1 and CD80 binding activity. An exemplary PD-L1 amino acid sequence is provided below (SEQ ID NO: 23).

By “anti-PD-L1 antibody” is meant an antibody that selectively binds a PD-L1 polypeptide. Exemplary anti-PD-L1 antibodies are described for example at U.S. Pat. No. 8,779,108 and U.S. Patent Application Publication No. 2014/0356353, which are herein incorporated by reference. Durvalumab (MEDI4736) is an exemplary anti-PD-L1 antibody. Other anti-PD-L1 antibodies include BMS-936559 (Bristol-Myers Squibb) and MPDL3280A (Roche).

By “PD-1 polypeptide” is meant a polypeptide or fragment thereof having at least about 85% amino acid identity to NCBI Accession No. NP_005009 and having PD-L1 binding activity. An exemplary PD-1 amino acid sequence is provided below (SEQ ID NO: 32).

By “PD-1 nucleic acid molecule” is meant a polynucleotide encoding a PD-1 polypeptide. An exemplary PD-1 nucleic acid molecule sequence is provided at NCBI Accession No. NM_005018.

The term “GITRL polypeptide” is meant to indicate a polypeptide or fragment thereof having at least about 85% amino acid identity to SEQ ID NO: 33 and having GITR binding activity. The term “GITRL” refers both to the full length GITRL and to soluble fragments, e.g., extracellular domain forms of GITRL resulting from proteolysis, and to monomeric forms of GITRL as well as oligomeric forms, e.g., trimeric GITRL. Amino acid sequences of membrane-bound and soluble forms of human GITRL are shown below.

The term “TNF-α polypeptide” is meant to indicate a polypeptide or fragment thereof having at least about 85% amino acid identity to NCBI Accession No. NP_000585.2 The term “TNF-α” refers both to the full length TNF-α and to soluble fragments, e.g., extracellular domain forms of TNF-α resulting from proteolysis, and to monomeric forms of TNF-α as well as oligomeric forms, e.g., trimeric TNF-α. Amino acid sequences of membrane-bound and soluble forms of human TNF-α are shown below.

As used herein, “OX40 polypeptide” means a polypeptide or fragment thereof having at least about 85% amino acid identity to NCBI Accession No. NP_003318. OX40 is a member of the TNFR-superfamily of receptors that is expressed on the surface of antigen-activated mammalian CD4+ and CD8+ T lymphocytes. OX40 receptor sequences are known in the art and are provided, for example, at GenBank Accession Numbers: AAB33944 or CAE11757.

The term “OX40L polypeptide” is meant to indicate a polypeptide or fragment thereof having at least about 85% amino acid identity to NCBI Accession No. NP_003317 and having OX40 binding activity. The term “OX40L” refers both to the full length OX40L and to soluble fragments, e.g., extracellular domain forms of OX40L resulting from proteolysis, and to monomeric forms of OX40L as well as oligomeric forms, e.g., trimeric OX40L. Amino acid sequences of membrane-bound and soluble forms of human OX40L are shown below.

The term “CD137L” is meant to indicate a polypeptide or fragment thereof having at least about 85% amino acid identity to NCBI Accession No. NP_001552.2 and having CD137 binding activity. The term “CD137L” refers both to the full length CD137L and to soluble fragments, e.g., extracellular domain forms of CD137L resulting from proteolysis, and to monomeric forms of CD137L as well as oligomeric forms, e.g., trimeric CD137L. Amino acid sequences of membrane-bound and soluble forms of human CD137L are shown below.

The term “antibody,” as used in this disclosure, refers to an immunoglobulin or a fragment or a derivative thereof, and encompasses any polypeptide comprising an antigen-binding site, regardless of whether it is produced in vitro or in vivo. The term includes, but is not limited to, polyclonal, monoclonal, monospecific, polyspecific, non-specific, humanized, single-chain, chimeric, synthetic, recombinant, hybrid, mutated, fusion proteins, and grafted antibodies. Unless otherwise modified by the term “intact,” as in “intact antibodies,” for the purposes of this disclosure, the term “antibody” also includes antibody fragments such as Fab, F(ab′)₂, Fv, scFv, Fd, dAb, and other antibody fragments and combinations thereof that retain antigen-binding function, i.e., the ability to bind, for example, PD-1 or PD-L1, specifically. Typically, such fragments would comprise an antigen-binding domain. Further, such fragments can be combined with others to form multi-antigen binding fusion proteins.

The terms “antigen-binding domain,” “antigen-binding fragment,” and “binding fragment” refer to a part of an antibody molecule that comprises amino acids responsible for the specific binding between the antibody and the antigen. In instances where an antigen is large, the antigen-binding domain may only bind to a part of the antigen. A portion of the antigen molecule that is responsible for specific interactions with the antigen-binding domain is referred to as an “epitope” or an “antigenic determinant.” An antigen-binding domain typically comprises an antibody light chain variable region (V_{L}) and an antibody heavy chain variable region (V_{H}), however, it does not necessarily include both. For example, a so-called “Fd” antibody fragment consists only of a V_{H} domain, but still retains some antigen-binding function of the intact antibody.

Binding fragments of an antibody are produced by recombinant DNA techniques, or by enzymatic or chemical cleavage of intact antibodies. Binding fragments include Fab, Fab′, F(ab′)2, Fv, and single-chain antibodies. An antibody other than a “bispecific” or “bifunctional” antibody is understood to have identical binding sites. Digestion of antibodies with the enzyme, papain, results in two identical antigen-binding fragments, known also as “Fab” fragments, and a “Fc” fragment, having no antigen-binding activity but having the ability to crystallize. Digestion of antibodies with the enzyme, pepsin, results in the F(ab′)2 fragment in which the two arms of the antibody molecule remain linked and comprise two-antigen binding sites. The F(ab′)2 fragment has the ability to crosslink antigen. “Fv” when used herein refers to the minimum fragment of an antibody that retains both antigen-recognition and antigen-binding sites. “Fab” when used herein refers to a fragment of an antibody that comprises the constant domain of the light chain and the CH1 domain of the heavy chain.

The term “mAb” refers to a monoclonal antibody. Antibodies of the disclosure comprise without limitation whole native antibodies, bispecific antibodies, chimeric antibodies, Fab, Fab′, single chain V region fragments (scFv), fusion polypeptides, unconventional antibodies, and combinations thereof.

In this disclosure, “comprises,” “comprising,” “containing,” and “having” and the like can have the meaning ascribed to them in U.S. Patent law and can mean “includes,” “including,” and the like; “consisting essentially of” or “consists essentially of” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

As used herein, the terms “determining,” “assessing,” “assaying,” “measuring,” and “detecting” refer to both quantitative and qualitative determinations, and as such, the term “determining” can be used interchangeably herein with “assaying,” “measuring,” and the like. Where a quantitative determination is intended, the phrase “determining an amount” of an analyte and the like is used. Where a qualitative and/or quantitative determination is intended, the phrase “determining a level” of an analyte or “detecting” an analyte is used.

As used herein, the term “Fc domain” domain refers to a portion of an antibody constant region. Traditionally, the term Fc domain refers to a protease (e.g., papain) cleavage product encompassing the paired CH2, CH3 and hinge regions of an antibody. In the context of this disclosure, the term Fc domain or Fc refers to any polypeptide (or nucleic acid encoding such a polypeptide), regardless of the means of production, that includes all or a portion of the CH2, CH3, and hinge regions of an immunoglobulin polypeptide.

The term “fusion polypeptide” or “fusion protein” refers to a polypeptide comprising two or more different polypeptides or active fragments thereof that are not naturally present in the same polypeptide. In various embodiments, the two or more different polypeptides are operatively linked together covalently, e.g., chemically linked or fused in frame by a peptide bond or a peptide linker. For example, a bispecific fusion protein can include one or more Fab and/or Fab′ fragments, an Fc fragment or region (such as, CH2 and CH3 without or without a hinge region), and/or a fusion protein attached to the one or more Fab and/or Fab′ fragments or the Fc fragment.

The terms “identical” or percent “identity,” in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity can be measured using sequence comparison software or algorithms or by visual inspection. Various algorithms and software are known in the art that can be used to obtain alignments of amino acid or nucleotide sequences (see e.g., Karlin et al., 1990, Proc. Natl. Acad. Sci., 87:2264-2268, as modified in Karlin et al., 1993, Proc. Natl. Acad. Sci., 90:5873-5877, and incorporated into the NBLAST and XBLAST programs (Altschul et al., 1991, Nucleic Acids Res., 25:3389-3402). In certain embodiments, Gapped BLAST can be used as described in Altschul et al., 1997, Nucleic Acids Res. 25:3389-3402. BLAST-2, WU-BLAST-2 (Altschul et al., 1996, Methods in Enzymology, 266:460-480), ALIGN, ALIGN-2 (Genentech, South San Francisco, Calif.) or Megalign (DNASTAR).

The term “isolated” refers to a molecule that is substantially free of other elements present in its natural environment. For instance, an isolated protein is substantially free of cellular material or other proteins from a cell or tissue source from which it is derived. The term “isolated” also refers to preparations where the isolated protein is sufficiently pure to be administered as a pharmaceutical composition, or at least 70-80% (w/w) pure, more preferably, at least 80-90% (w/w) pure, even more preferably, 90-95% pure; and most preferably, at least 95%, 96%, 97%, 98%, 99%, or 100% (w/w) pure.

The term “reference” refers to a standard of comparison.

The term “specifically binds” refers to an agent (e.g., CD40L, GITRL, OX40L, or CD137L) that recognizes and binds a molecule (e.g., CD40 polypeptide, GITR polypeptide, OX40 polypeptide, or CD137 polypeptide, respectively), but which does not substantially recognize and bind other molecules in a sample, such as a biological sample. For example, two molecules that specifically bind form a complex that is relatively stable under physiologic conditions. Specific binding is characterized by a high affinity and a low to moderate capacity as distinguished from nonspecific binding, which usually has a low affinity with a moderate to high capacity.

The term “subject” refers to a mammal, including, but not limited to, a human or non-human mammal, such as a bovine, equine, canine, ovine, or feline.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” treating,” “treatment,” and the like, refer to reducing and/or ameliorating a disorder and/or symptoms associated therewith. It will be appreciated that, although not precluded, treating a disorder or condition does not require that the disorder, condition, or symptoms associated therewith be completely eliminated. For example, as contemplated herein, treatment of a disorder includes preventing the exacerbation of symptoms of the disorder.

As used herein, the term “or” is understood to be inclusive unless specifically stated or obvious from context to the contrary. As used herein, the terms “a”, “an”, and “the” are understood to be singular or plural unless specifically stated or obvious from context to the contrary.

Furthermore, “and/or” where used herein is to be taken as specific disclosure of each of the two or more specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; and A (alone); B (alone); and C (alone).

Unless specifically stated or obvious from context, as used herein, the term “about” is understood as within a range of normal tolerance in the art, for example within 2 standard deviations of the mean. “About” can be understood as within greater or less than 10%, 9%, 8%, 7%, 6%, 5%, 4%, 3%, 2%, 1%, 0.5%, 0.1%, 0.05%, or 0.01% of a stated value. Unless indicated otherwise, all numerical values provided herein are considered to be implicitly modified by the term “about.”

The recitation of a listing of chemical groups in any definition of a variable herein includes definitions of that variable as any single group or any combination of listed groups. The recitation of an embodiment for a variable or aspect herein includes that embodiment as any single embodiment or in any combination with any other embodiments or portions thereof.

Any compositions or methods provided herein can be combined with one or more of any of the other compositions and methods provided herein.

### Overview

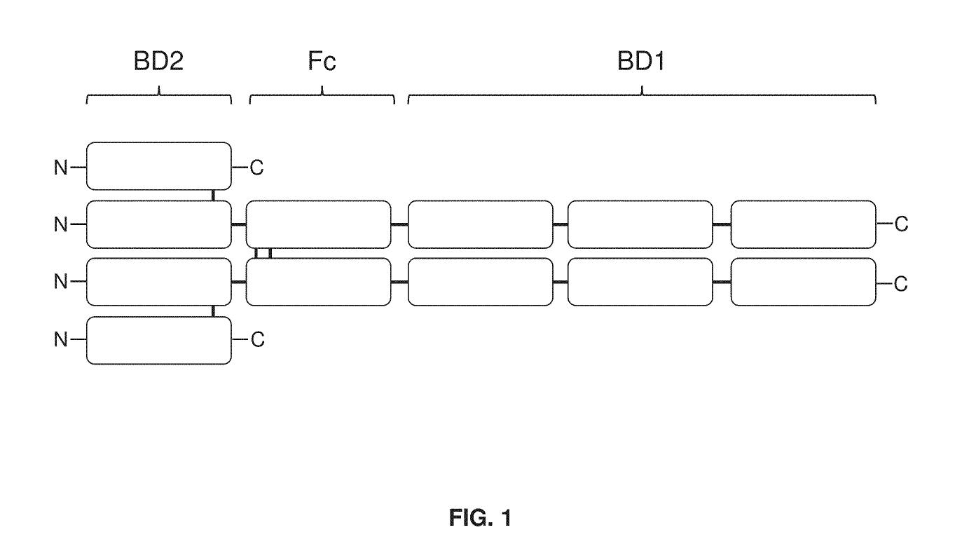

The present disclosure provides several fusion proteins (FP), and in particular, bispecific fusion proteins (BFPs) that are multivalent and designed to control T cell-mediated anticancer immune responses. In one non-exclusive embodiment, contemplated BFPs include a first binding domain (BD1), a second binding domain (BD2), and an immunoglobulin Fc region including, for example, CH2 and CH3 domains (see FIG. 1). BD1 and BD2 can each separately include one or more of an antigen binding domain, an antigen binding fragment, a binding fragment, a receptor agonist, antagonist, or ligand, and the like. In one particular example, at least one of BD1 and BD2 is a Fab domain, an scFv, a single domain antibody, and an antibody variable domain.

In some embodiments, BD1, BD2, and the Fc region of some BFPs can be linked together by one or more linkers, such as a peptide linker. In this way, in some embodiments, contemplated BFPs can be genetically encoded, expressed a single chain fusion protein (scfp) that can be expressed and assembled within a host cell.

In some embodiments, BD1 and BD2 binding domains each retain similar binding capabilities and functions toward their respective targets (e.g., epitopes, proteins, and/or receptors) as their parental (native, unbound) constituent components. In one embodiment, the bispecific binding capacity of contemplated BFPs allows a single BFP to simultaneously engage and/or bind to two molecular targets on a single cell surface (i.e., a cis-interaction) or on cell surfaces of adjacent cells (i.e., a trans-interaction). In some embodiments, BFPs are contemplated that can cause cis- and trans-interactions either separately or at the same time.

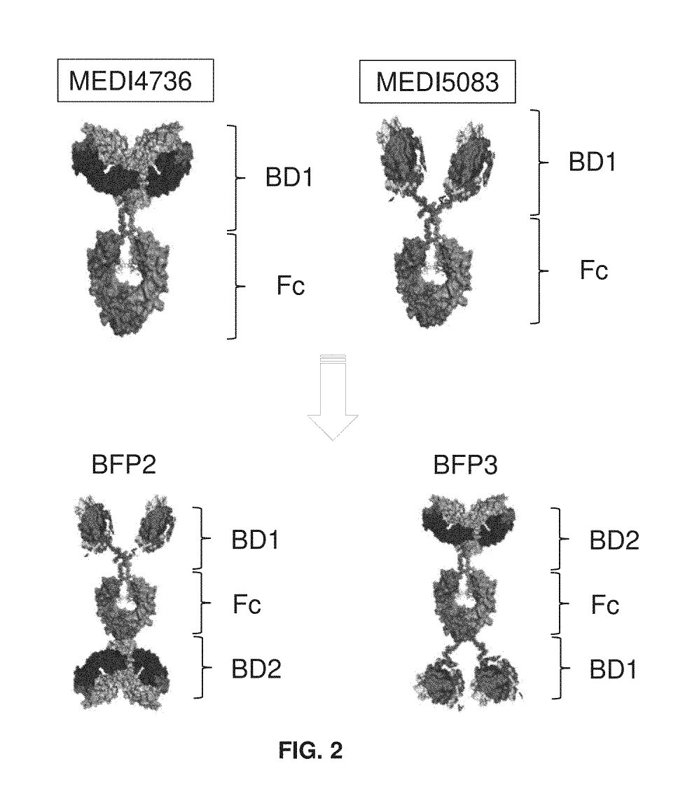

As contemplated herein, different BFP configurations are possible, such as are seen in FIG. 2 and Table 1 below.

TABLE 1
Exemplary Bispecific Fusion Proteins.

| | | | BD1 | | | |
|---|---|---|---|---|---|---|
| Name(s) | Format | BD1 | target | BD2 | BD2 target | Fc |
| CD40L FP- | BFP2 | 6 units of | CD40 | 1 unit of | PD-L1 | IgG4P |
| anti-PD-L1 | | CD40L FP | | F(ab)2 anti- | | |
| | | | | PD-L1 | | |
| | | | | (from | | |
| | | | | MEDI4736) | | |
| Anti-PD-L1- | BFP3 | 6 units of | CD40 | 1 unit of | PD-L1 | IgG4P |
| CD40L FP | | CD40L FP | | F(ab)2 anti- | | |
| (MEDI7526) | | | | PD-L1 | | |
| | | | | (from | | |
| | | | | MEDI4736) | | |
| Anti-PD-L1- | BFP3 | 6 units of | murine | 1 unit of | murine | murine |
| CD40L FP | | murine CD40L | CD40 | F(ab)2 anti- | PD-L1 | IgG1D265A |
| (murine | | FP | | murine PD- | | |
| surrogate for | | | | L1 | | |
| MEDI7526) | | | | | | |
| Anti-PD-L1- | BFP3 | 6 units of TNF- | TNF-α | 1 unit of | PD-L1 | IgG4P |
| TNF-α FP | | α FP | receptors | F(ab)2 anti- | | |
| | | | | PD-L1 | | |
| | | | | (from | | |
| | | | | MEDI4736) | | |
| Anti-PD1- | BFP3 | 6 units of | OX40 | 1 unit of | PD1 | IgG4P |
| OX40L FP | | OX40L FP | | F(ab)2 anti- | | |
| | | | | PD1 | | |
| Anti-PD1- | BFP3 | 4 units of | OX40 | 1 unit of | PD1 | IgG4P |
| OX40L FP | | OX40L wild | | F(ab)2 anti- | | |
| 2xWT | | type, 2 unites | | PD1 | | |
| | | of OX40L | | | | |
| | | F180A FP | | | | |
| Anti-PD1- | BFP3 | 2 units of | OX40 | 1 unit of | PD1 | IgG4P |
| OX40L FP | | OX40L wild | | F(ab)2 anti- | | |
| 1xWT | | type, 4 unites | | PD1 | | |
| | | of OX40L | | | | |
| | | F180A FP | | | | |
| Anti-PD-L1- | BFP3 | 6 units of | OX40 | 1 unit of | PD1 | IgG4P |
| OX40L FP | | OX40L FP | | F(ab)2 anti- | | |
| (MEDI5615) | | | | PD1 | | |
| Anti-PD1- | BFP3 | 6 units of | CD137 | 1 unit of | PD1 | IgG4P |
| CD137L FP | | CD137L FP | | F(ab)2 anti- | | |
| | | | | PD1 | | |
| Anti-PD1- | BFP3 | 6 units of | GITR | 1 unit of | PD1 | IgG4P |
| GITRL FP* | | GITRL FP | | F(ab)2 anti- | | |
| (MEDI3387, | | | | PD1 | | |
| BIOAE003, | | | | | | |
| BFP@- | | | | | | |
| PD1(0115)- | | | | | | |
| GITRL(sc)- | | | | | | |
| G4P, PD- | | | | | | |
| 1/GITRL | | | | | | |
| BFP IgG4P) | | | | | | |
| Anti-PD1- | BFP3 | 6 units of | GITR | 1 unit of | PD1 | IgG1 |
| GITRL FP* | | GITRL FP | | F(ab)2 anti- | | |
| (MEDI5771, | | | | PD1 | | |
| BIOAE005, | | | | | | |
| PD-1/GITRL | | | | | | |
| BFP IgG1) | | | | | | |

In one particular embodiment, BFPs of any format disclosed herein are contemplated that incorporate as at least one binding domain that targets any TNF superfamily member paired with another binding domain that targets any other cell surface protein. Additional examples of BFP binding domains TNF superfamily members contemplated herein include LIGHT, CD30L, CD27L, and TL1a, which can be incorporated into a BFP2 or BFP3 format.

### Mechanism of Action

In some preferred embodiments, the disclosure features BFP3 format bispecific fusion proteins including one or more N-terminus antigen binding subunits, a central Fc polypeptide core, and one or more C-terminus ligand proteins. In one embodiment, the one or more N-terminus antigen binding subunits (BD2) can be anti-PD-1 and/or anti-PD-1L antigen binding subunits, the central Fc polypeptide core can be an IgG1 or IgG4 Fc region polypeptide (CH2 and CH3), and the one or more C-terminus ligand proteins (BD1) can include, for example, GITRL, OX40L, CD40L, TNF-α, and/or CD137.

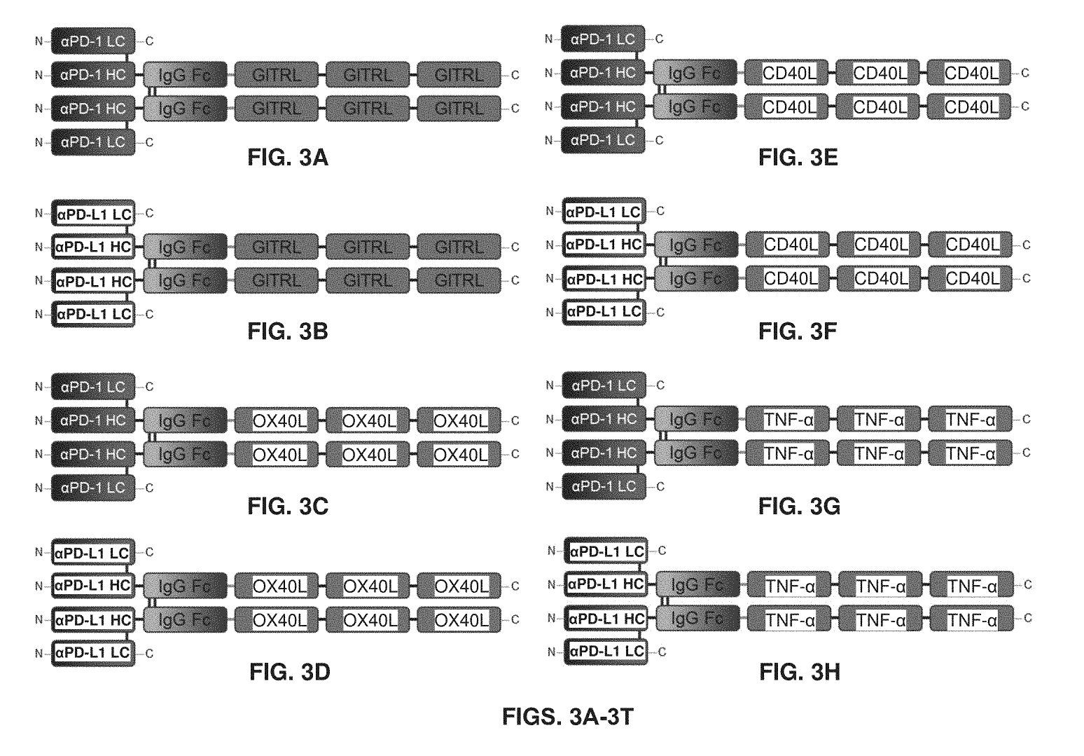

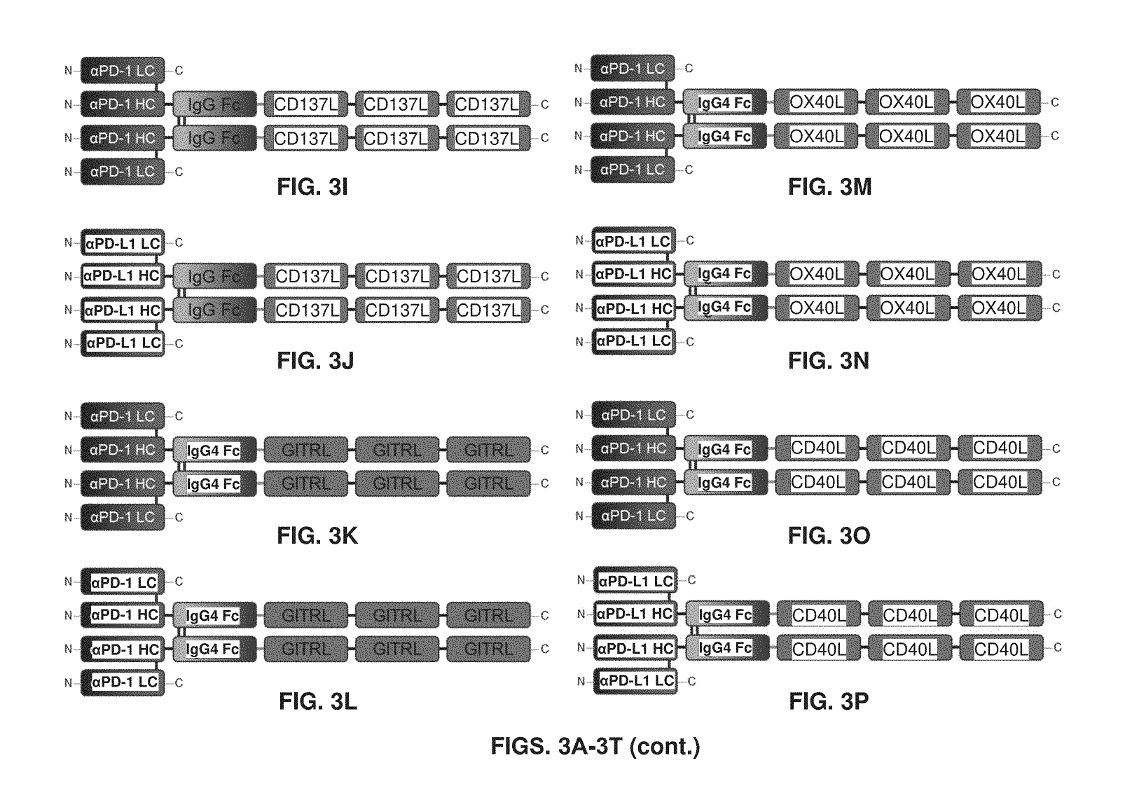

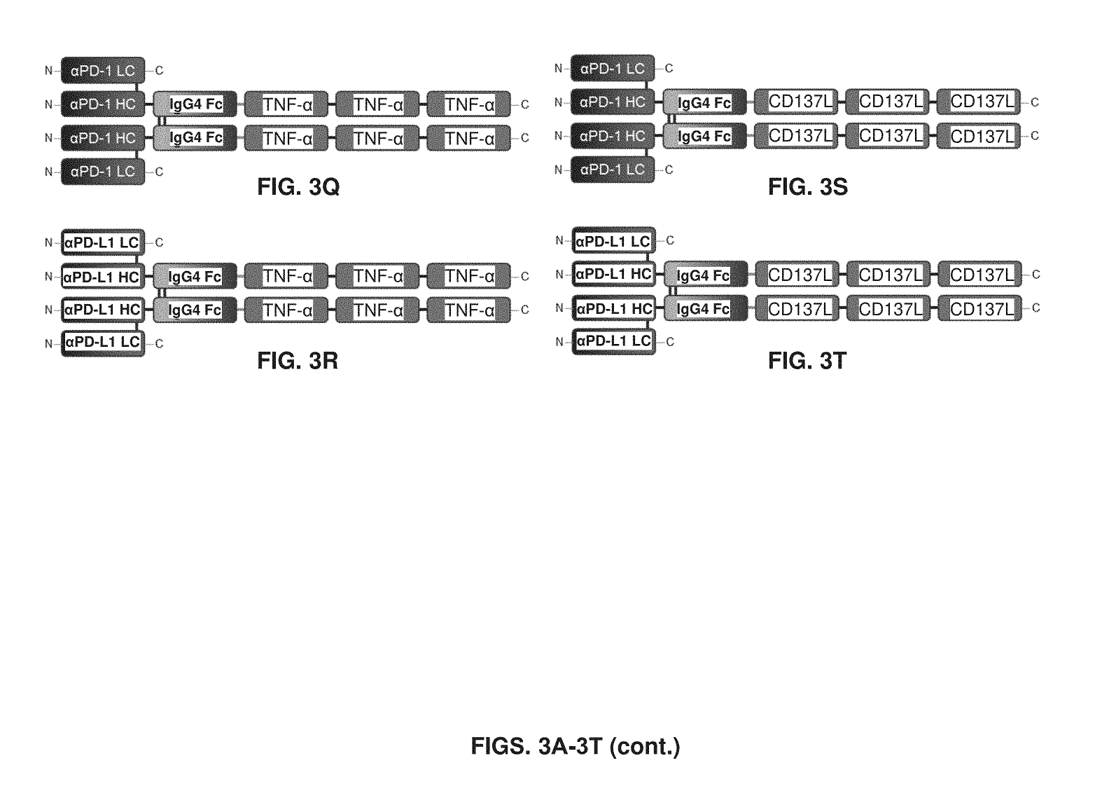

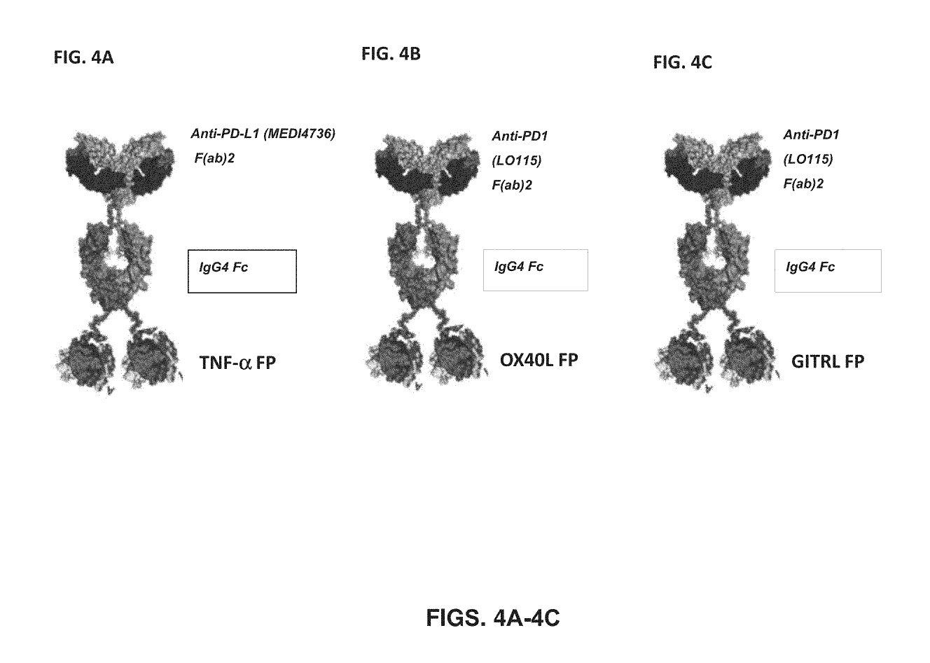

In one embodiment, the BFP3 format shown in FIGS. 1-4 allows co-opting BD1 and BD2 targets when present on different cells (trans-interaction), resulting in the activation of downstream signaling pathways linked to Fcγ receptors on myeloid cells. Using MEDI7526 (see Table 1) as an example, FcγRI engagement, in context of CD40 stimulation, can drive greater than additive NF-κB activation.

In some embodiments, binding of BD1 to its target (e.g., a cell receptor) can trigger internalization of the binding complex and initiate a signaling cascade (e.g., promoting T-cell activation and/or replication) or inhibit a signaling cascade (e.g., removing an inhibitory blockade). For example, BD1 binding and internalization causes BD2 and its target, e.g., either PD1 or PD-L1 to be internalized within a cell. Forced internalization of PD1/PD-L1 triggers their degradation, leading to a long period of absence of PD1/PD-L1 on the cell surface. This is a novel approach to attenuate PD1/PD-L1 inhibitory functions by removing PD1/PDL1 from the cell surface to promote a T cell-mediated immune response.

In one embodiment, contemplated BFPs are dimerized single-chain fusion protein backbone subunits, and for example, include two, identical single-chain fusion proteins joined via disulfide bonds. Individual components of each single-chain fusion protein can be linked via peptide linkers. Contemplated peptide linkers can be of any length that allows functional formation of the desired bispecific fusion protein, such as 1, 2, 3, 4, 5, 6, 7, 8, 9, 10, 11, 12, 13, 14, 15, 16, 17, 18, 19, or 20 or more amino acids. In one particular embodiment, peptide linkers having a length of 9 amino acids or more between the individual components of the contemplated fusion proteins are contemplated. It has been found that peptide linkers of 9 or more amino acids in contemplated bispecific fusion proteins retained stability and/or did not cause aggregation of such fusion proteins. In fact, a wide range of linker lengths has proven to be well tolerated in the bispecific fusion proteins of the present disclosure than has been suggested elsewhere.

Single chain bispecific fusion proteins of the disclosure have been shown to be stable and to exhibit bioactivity. As described herein, bispecific fusion protein subunit stability and activity was due at least in part to the length of the linkers used in the bispecific fusion proteins of the disclosure. Linkers greater than 9 amino acids in length did not present any significant issues with aggregation and/or stability. The bispecific fusion proteins of the disclosure also provide other features and advantages of single chain Fc proteins.

### BD1 Antigen Binding Domain

Any TNF-α family member that can be used to control a T cell-mediated immune response is contemplated for use herein. Specific examples of contemplated TNF-α family members include CD40L, GITRL, OX40L, TNF-α, and CD137L. It is known that naturally occurring soluble cytokine members of the TNF ligand family exhibit their bioactivity as homotrimers. However, trimeric complexes of TNF ligands tend to denature via dissociation of their monomers and are difficult to prepare from recombinant monomeric units. To prevent the dissociation of the homotrimers into monomers at least three monomers of a TNF ligand are covalently linked to one another via their C terminals and N terminals by means of peptide linkers to form a “single-chain (sc)” molecule. Therefore, the entire molecule (at least three monomers of a member of the TNF ligand family with the two peptide linkers) consists of a single protein strand, so that dissociation into monomers can no longer occur.

In addition, fusion of the TNF ligand to an Fc domain, as in the single-chain fusion proteins of the disclosure, may be used to obtain dimerization trimers. The dimerization of soluble domains is accomplished by assembly of two Fc-domains via disulfide bridges. The local enrichment of single chain TNF ligands on cells or neighboring cells has the potential to increase the bioactivity of these fusion proteins.

### BD2 Antigen Binding Domain

Antigen binding regions, such as Fab fragments, that selectively bind PD-1 and PD-L1, and inhibit the binding or activation of PD-1 and PD-L1 are useful in the BFPs of the disclosure. Fab (Fragment antigen-binding) fragments consist of the VH-CH1 and VL-CL domains covalently linked by a disulfide bond between the constant regions. To overcome the tendency of non-covalently linked VH and VL domains in the Fv to dissociate when co-expressed in a host cell, a so-called single chain (sc) Fv fragment (scFv) can be constructed. In a scFv, a flexible and adequately long polypeptide links either the C-terminus of the VH to the N-terminus of the VL or the C-terminus of the VL to the N-terminus of the VH. In some embodiments, linker peptides contemplated herein include a multimer of a GGGGS (Gly4Ser) peptide but other linkers are also known in the art and can be used herein. For example, a possible linker is a 15-residue (Gly4Ser)3 peptide.

BD2 antigen binding domains of the disclosure (e.g., specific for anti-PD-1 or anti-PD-L1) can optionally comprise antibody constant regions or parts thereof. For example, a VL domain may have attached, at its C terminus, antibody light chain constant domains including human Cκ or Cλ, chains. Similarly, a specific antigen-binding domain based on a VH domain may have attached all or part of an immunoglobulin heavy chain derived from any antibody isotope, e.g., IgG, IgA, IgE, and IgM and any of the isotope sub-classes, which include but are not limited to, IgG1 and IgG4.

One of ordinary skill in the art will recognize that the BD2 antigen binding domain of BFPs of this disclosure can be used to detect, measure, and inhibit proteins that differ somewhat from PD-1 and PD-L1. The BD2 antigen binding domain is expected to retain the specificity of binding so long as the target protein comprises a sequence which is at least about 60%, 70%, 80%, 90%, 95%, or more identical to any sequence of at least 100, 80, 60, 40, or 20 of contiguous amino acids described herein. The percent identity is determined by standard alignment algorithms such as, for example, Basic Local Alignment Tool (BLAST) described in Altshul et al. (1990) J. Mol. Biol., 215: 403-410, the algorithm of Needleman et al. (1970) J. Mol. Biol., 48: 444-453, or the algorithm of Meyers et al. (1988) Comput. Appl. Biosci., 4: 11-17.

In addition to sequence homology analyses, epitope mapping (see, e.g., Epitope Mapping Protocols, ed. Morris, Humana Press, 1996) and secondary and tertiary structure analyses can be carried out to identify specific 3D structures assumed by the disclosed BD2 antigen binding domains and their complexes with antigens. Such methods include, but are not limited to, X-ray crystallography (Engstom (1974) Biochem. Exp. Biol., 11:7-13) and computer modeling of virtual representations of the presently disclosed antibodies (Fletterick et al. (1986) Computer Graphics and Molecular Modeling, in Current Communications in Molecular Biology, Cold Spring Harbor Laboratory, Cold Spring Harbor, N.Y.).

Contemplated anti-PD-L1 antigen binding domains can be taken from or derived from durvalumab (MEDI4736), an exemplary anti-PD-L1 antibody that is selective for PD-L1 and blocks the binding of PD-L1 to the PD-1 and CD80 receptors. Durvalumab can relieve PD-L1-mediated suppression of human T-cell activation in vitro and inhibits tumor growth in a xenograft model via a T-cell dependent mechanism. Information regarding durvalumab (or fragments thereof) for use in the methods provided herein can be found in U.S. Pat. Nos. 8,779,108 and 9,493,565 the disclosures of which are incorporated herein by reference in their entireties.

In a specific aspect, an antigen-binding fragment of durvalumab for use herein includes a heavy chain variable region and a light chain variable region, wherein the heavy chain variable region comprises the Kabat-defined CDR1, CDR2, and CDR3 sequences shown herein above, and wherein the light chain variable region comprises the Kabat-defined CDR1, CDR2, and CDR3 sequences shown herein above. Those of ordinary skill in the art would easily be able to identify Chothia-defined, Abm-defined, or other CDR definitions known to those of ordinary skill in the art. In a specific aspect, an antigen-binding fragment of durvalumab for use includes the variable heavy chain and variable light chain CDR sequences of the 2.14H9OPT antibody as disclosed in U.S. Pat. No. 8,779,108.

Contemplated anti-PD-1 antigen binding domains can be taken from or derived from LO115, an exemplary anti-PD-1 antibody that is selective for PD-1 and blocks the binding of PD-1 to the PD-L1 and PD-L2 receptors.

### Fc Region

In some embodiments, the present disclosure provides bispecific fusion proteins with an IgG1 or IgG4 Fc region polypeptide that can have at least one amino acid modification. For example, an amino acid can be substituted at one or more positions selected from 228 and 235 as numbered by the EU index as set forth in Kabat. For example, the Fc region can be an IgG4 Fc region and variant amino acids are one or more of 228P (giving rise to “IgG4P”), 235E, and 235Y as numbered by the EU index as set forth in Kabat. As another example, an IgG1 Fc region is contemplated with variant amino acids that can include one or more of L234F/L235E/P331S (referred to herein elsewhere as “IgG TM”). All IgG4 molecules disclosed herein, whether labeled IgG4 or IgG4P, contain the 228P mutation

In one embodiment, the fragment crystallizable (Fc) domain used herein is of durvalumab, which contains the triple mutation in the constant domain of the IgG1 heavy chain that reduces binding to the complement component C1 q and the Fcγ receptors responsible for mediating antibody-dependent cell-mediated cytotoxicity (ADCC).

### Linkers

Subunits in the bispecific fusion proteins of the disclosure can be connected by polypeptide linkers, wherein each linker is fused and/or otherwise connected (e.g., via a peptide bond) to at least two polypeptides or subunits. Combinations of linkers in the bispecific fusion proteins can be homomeric or heteromeric. In some embodiments, the amino acid sequences of all peptide linkers present in a bispecific fusion protein of the disclosure are identical. In other embodiments, the amino acid sequences of at least two of the peptide linkers present in a bispecific fusion protein of the disclosure are different. A linker polypeptide should have a length, which is adequate to link two or more monomer subunits in such a way that they assume the correct conformation relative to one another so that they retain their desired activity. The use of naturally occurring as well as artificial peptide linkers to connect polypeptides into novel linked fusion polypeptides is well known in the literature. Accordingly, the linkers fusing two or more monomer subunits can be natural linkers, artificial linkers, or combinations thereof.

As described herein, it has been found that peptide linkers having a length of 9 amino acids or more between the fusion protein subunits retained stability and/or did not cause excessive aggregation of such fusion proteins. Thus, it is envisioned that in some embodiments the polypeptide linker can include about 9 to about 20 amino acids residues, about 9 to about 15 amino acid residues, or about 9 amino acid residues. The amino acid residues selected for inclusion in the polypeptide linker should exhibit properties that do not interfere significantly with the activity or function of the fusion protein subunits of the disclosure. Thus, a polypeptide linker should not, on the whole, exhibit a charge which would be inconsistent with the activity or function of a particular fusion protein subunit of the disclosure, or interfere with internal folding, or form bonds or other interactions with amino acid residues in one or more of the monomer subunits which would seriously impede the binding.

In various embodiments, a polypeptide linker possesses conformational flexibility. Suitable flexible linkers include, for example, those having a combination of Gly and Ser residues, where the ratio of Gly to Ser is ≥1. In some embodiments, a polypeptide linker is an inherently unstructured natural or artificial polypeptide (see, e.g., Schellenberger et al., Nature Biotechnol. 27:1186-1190, 2009; see also, Sickmeier et al., Nucleic Acids Res. 35:D786-93, 2007).

In certain specific embodiments, a linker between bispecific fusion protein subunits can be a multimer of GGGGS, such as GGGGSGGGGSGGGGSGGGGS (SEQ ID NO: 39), GGGGSGGGGSGGGGS (SEQ ID NO: 40), or GGGGSGGGGS (SEQ ID NO: 41). In other embodiments, a contemplated linker can be GGGGSGGGS (SEQ ID NO: 42).

### Specific Embodiments

In specific embodiments, bispecific fusion proteins disclosed herein contain a pair of single chain fusion proteins each comprising, from N-terminus to C-terminus, an anti-PD-1 or anti-PD-L1 Fab fragment comprising a light chain variable region and a heavy chain variable region, covalently linked to an (b) IgG1 or IgG4P Fc polypeptide covalently linked to (c) a first peptide linker covalently linked to a first TNF superfamily ligand subunit, covalently linked to a second peptide linker, covalently linked to a second TNF superfamily ligand subunit, covalently linked to a third peptide linker, covalently linked to a third TNF superfamily ligand subunit.

### Derivatives

Polypeptides (e.g., anti-PD-1 Fab, anti-PD-L1 Fab, GITRL, OX40L, CD40L, TNF-α, or CD137L) of the disclosure can include variants of the sequences provided that retain the ability to specifically bind their targets. Such variants can be derived from the sequence of these polypeptides by a skilled artisan using techniques well known in the art. For example, amino acid substitutions, deletions, or additions, can be made in the FRs and/or in the CDRs of the anti-PD-1 or PD-L1 Fab fragments. While changes in the FRs are usually designed to improve stability and immunogenicity of the antigen binding domain, changes in the CDRs are typically designed to increase affinity of the antigen binding domain for its target. Variants of FRs also include naturally occurring immunoglobulin allotypes. Such affinity-increasing changes may be determined empirically by routine techniques that involve altering the CDR and testing the affinity of the antigen binding domain for its target. For example, conservative amino acid substitutions can be made within any one of the disclosed CDRs. Various alterations can be made according to the methods described in Antibody Engineering, 2nd ed., Oxford University Press, ed. Borrebaeck, 1995. These alterations include but are not limited to nucleotide sequences that are altered by the substitution of different codons that encode a functionally equivalent amino acid residue within the sequence, thus producing a “silent” change. For example, the nonpolar amino acids include alanine, leucine, isoleucine, valine, proline, phenylalanine, tryptophan, and methionine. The polar neutral amino acids include glycine, serine, threonine, cysteine, tyrosine, asparagine, and glutamine. The positively charged (basic) amino acids include arginine, lysine, and histidine. The negatively charged (acidic) amino acids include aspartic acid and glutamic acid.

Derivatives and analogs of polypeptides and/or antibodies of the disclosure can be produced by various techniques well known in the art, including recombinant and synthetic methods (Maniatis (1990) Molecular Cloning, A Laboratory Manual, 2nd ed., Cold Spring Harbor Laboratory, Cold Spring Harbor, N.Y., and Bodansky et al. (1995) The Practice of Peptide Synthesis, 2nd ed., Spring Verlag, Berlin, Germany).

In one embodiment, a method for making a VH domain which is an amino acid sequence variant of a VH domain of the disclosure comprises a step of adding, deleting, substituting, or inserting one or more amino acids in the amino acid sequence of the presently disclosed VH domain, optionally combining the VH domain thus provided with one or more VL domains, and testing the VH domain or VH/VL combination or combinations for specific binding to the antigen. An analogous method can be employed in which one or more sequence variants of a VL domain disclosed herein are combined with one or more VH domains.

Analogous shuffling or combinatorial techniques are also disclosed by Stemmer (Nature (1994) 370: 389-391), who describes the technique in relation to a β-lactamase gene but observes that the approach may be used for the generation of antibodies.

In further embodiments, one may generate novel VH or VL regions carrying one or more sequences derived from the sequences disclosed herein using random mutagenesis of one or more selected VH and/or VL genes. One such technique, error-prone PCR, is described by Gram et al. (Proc. Nat. Acad. Sci. U.S.A. (1992) 89: 3576-3580).

Another method that may be used is to direct mutagenesis to CDRs of VH or VL genes. Such techniques are disclosed by Barbas et al. (Proc. Nat. Acad. Sci. U.S.A. (1994) 91: 3809-3813) and Schier et al. (J. Mol. Biol. (1996) 263: 551-567).

Similarly, one, two, or all three CDRs of an antigen binding domain may be grafted into a repertoire of VH or VL domains, which are then screened for an antigen-binding fragment specific for PD-1 or PD-L1.

A portion of an immunoglobulin variable domain useful herein can comprise at least one of the CDRs substantially as set out herein and, optionally, intervening framework regions from the scFv fragments as set out herein. The portion may include at least about 50% of either or both of FR1 and FR4, the 50% being the C-terminal 50% of FR1 and the N-terminal 50% of FR4. Additional residues at the N-terminal or C-terminal end of the substantial part of the variable domain can be those not normally associated with naturally occurring variable domain regions. For example, construction of antibodies by recombinant DNA techniques can result in the introduction of N- or C-terminal residues encoded by linkers introduced to facilitate cloning or other manipulation steps. Other manipulation steps include the introduction of linkers to join variable domains to further protein sequences including immunoglobulin heavy chain constant regions, other variable domains (for example, in the production of diabodies), or proteinaceous labels as discussed in further detail below.

Antigen binding domains of the disclosure (e.g., anti-PD-1 and/or anti-PD-L1) described herein can be linked to another functional molecule, e.g., another peptide or protein (albumin, another antibody, etc.). For example, the antigen binding domains can be linked by chemical cross-linking or by recombinant methods. The antigen binding domains can also be linked to one of a variety of nonproteinaceous polymers, e.g., polyethylene glycol, polypropylene glycol, or polyoxyalkylenes, in the manner set forth in U.S. Pat. Nos. 4,640,835; 4,496,689; 4,301,144; 4,670,417; 4,791,192; or 4,179,337. The antigen binding domains can be chemically modified by covalent conjugation to a polymer, for example, to increase their circulating half-life. Exemplary polymers and methods to attach them are also shown in U.S. Pat. Nos. 4,766,106; 4,179,337; 4,495,285, and 4,609,546.

The disclosed antibody fragments can also be altered to have a glycosylation pattern that differs from the native pattern. For example, one or more carbohydrate moieties can be deleted and/or one or more glycosylation sites added. Addition of glycosylation sites to the presently disclosed antibody fragments can be accomplished by altering the amino acid sequence to contain glycosylation site consensus sequences known in the art. Another means of increasing the number of carbohydrate moieties on the antibody fragments is by chemical or enzymatic coupling of glycosides to the amino acid residues of the antibody. Such methods are described in WO 87/05330, and in Aplin et al. (1981) CRC Crit. Rev. Biochem., 22: 259-306. Removal of any carbohydrate moieties from the antibodies may be accomplished chemically or enzymatically, for example, as described by Hakimuddin et al. (1987) Arch. Biochem. Biophys., 259: 52; and Edge et al. (1981) Anal. Biochem., 118: 131 and by Thotakura et al. (1987) Meth. Enzymol., 138: 350. The antibody fragments may also be tagged with a detectable, or functional, label. Detectable labels include radiolabels such as 1311 or 99Tc, which may also be attached to antibody fragments using conventional chemistry. Detectable labels also include enzyme labels such as horseradish peroxidase or alkaline phosphatase. Detectable labels further include chemical moieties such as biotin, which may be detected via binding to a specific cognate detectable moiety, e.g., labeled avidin.

Antigen binding domains, in which CDR sequences differ only insubstantially from those set forth herein are encompassed within the scope of this disclosure. Typically, an amino acid is substituted by a related amino acid having similar charge, hydrophobic, or stereochemical characteristics. Such substitutions would be within the ordinary skills of an artisan. Unlike in CDRs, more substantial changes can be made in FRs without adversely affecting the binding properties of an antibody. Changes to FRs include, but are not limited to, humanizing a non-human derived or engineering certain framework residues that are important for antigen contact or for stabilizing the binding site, e.g., changing the class or subclass of the constant region, changing specific amino acid residues which might alter the effector function such as Fc receptor binding, e.g., as described in U.S. Pat. Nos. 5,624,821 and 5,648,260 and Lund et al. (1991) J. Immun. 147: 2657-2662 and Morgan et al. (1995) Immunology 86: 319-324, or changing the species from which the constant region is derived.

One of skill in the art will appreciate that the modifications described above are not all-exhaustive, can be applied to the protein subunits described herein, and that many other modifications would be possible for a skilled artisan in light of the teachings of the present disclosure.

### Bispecific Fusion Protein Production

The practice of the present disclosure employs, unless otherwise indicated, techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polypeptides of the disclosure, and, as such, may be considered in making and practicing the disclosure. Particularly useful techniques for particular embodiments will be discussed in the Examples.

### Assaying Bispecific Fusion Protein Properties and Activities

Stability of the bispecific fusion protein subunits of the disclosure, isolated or as part of a multimer, can be measured readily by techniques well known in the art, such as thermal (Tₘ) and chaotropic denaturation (such as treatment with urea, or guanidine salts), protease treatment (such as treatment with thermolysin) or another art accepted methodology to determine protein stability. A comprehensive review of techniques used to measure protein stability can be found, for example in “Current Protocols in Molecular Biology” and “Current Protocols in Protein Science” by John Wiley and Sons. 2007.

The binding affinity and other binding properties of a bispecific fusion proteins according to present disclosure can be determined by a variety of in vitro assay methods known in the art including for example, equilibrium methods (e.g., enzyme-linked immunoabsorbent assay (ELISA) or kinetics (e.g., BIACORE® analysis), and other methods such as indirect binding assays, competitive binding assays, gel electrophoresis, and chromatography (e.g., gel filtration). These and other methods can utilize a label on one or more of the components being examined and/or employ a variety of detection methods including but not limited to chromogenic, fluorescent, luminescent, or isotopic labels. A detailed description of binding affinities and kinetics can be found in Paul, W.E., ed., Fundamental Immunology, 4th Ed., Lippincott-Raven, Philadelphia (1999).

Additional in vitro and in vivo methods for determining the function or activity of bispecific fusion protein are described herein. These assays may be used to determine one or more of an immune response (e.g., one or more of T-cell function and memory, B-cell activation or proliferation, dendritic cell maturation or activation, Thl cytokine or chemokine response, monocyte-derived macrophage M1/M2 polarization, antigen presentation and/or immunosuppression of a tumor microenvironment). In vivo, various animal models for assaying anti-cancer or anti-tumor activity are known in the art, including for example, the B16-F10 tumor mouse model. Additional, methods of assessing pharmacodynamic and pharmacokinetic properties are also well-known.

### Anti-Tumor Therapy

The present disclosure also features compositions and methods that are useful for treating cancer comprising a bispecific fusion protein, such as described above. In various embodiments, the bispecific fusion proteins can be administered in combination with other anticancer drugs or drugs that augment immune cell responses to cancer.

Further provided herein are methods for treating cancer including administration of one or more bispecific fusion proteins, such as those shown in FIGS. 1-4. As shown herein, administration of bispecific fusion proteins can result in a reduction in tumor volume in, e.g., a mouse tumor model. In certain aspects, a patient presenting with a solid tumor is administered a bispecific fusion protein.

Treatment with a cancer therapy including a bispecific fusion protein causes, for example, a reduction in the rate of progression of the cancer, a retardation or stabilization of tumor growth, tumor shrinkage, and/or tumor regression. In some aspects, the reduction or retardation of tumor growth can be statistically significant. A reduction in tumor growth can be measured by comparison to the growth of patient's tumor at baseline, against an expected tumor growth, against an expected tumor growth based on a large patient population, or against the tumor growth of a control population.

In other embodiments, the methods of the disclosure increase cancer survival rates and extend life. For example, data disclosed herein demonstrate not only the effectiveness of BFP molecules for treating cancer, but that use of a BFP (MEDI7526) results in lower toxicity than treatment with a combination of its parental reagents (Durva+MEDI5083). Therefore, use of BFP molecules for cancer treatment can achieve an effective anticancer response with less toxicity and improve overall patient health. In other words, use of the bispecific fusion proteins disclosed herein can provide treatment results that are not achievable with the monotherapies alone.

Clinical response to administration of a cancer therapy can be assessed using diagnostic techniques known to clinicians, including but not limited to magnetic resonance imaging (MRI) scan, x-radiographic imaging, computed tomographic (CT) scan, flow cytometry or fluorescence- activated cell sorter (FACS) analysis, histology, gross pathology, and blood chemistry, including but not limited to changes detectable by ELISA, RIA, and chromatography.

The following examples are put forth so as to provide those of ordinary skill in the art with a complete disclosure and description of how to make and use the assay, screening, and therapeutic methods of the disclosure, and are not intended to limit the scope of what the inventors regard as their disclosure.

### EXAMPLES

The disclosure is now described with reference to the following examples. These examples are illustrative only and the disclosure should in no way be construed as being limited to these examples but rather should be construed to encompass any and all variations which become evident as a result of the teachings provided herein.

### Example No. 1. Generation of Bispecific Fusion Proteins

Bispecific fusion proteins tested in the examples below were constructed using a single chain fusion protein (scfp) construct of three ligand subunits (primarily corresponding to the TNF homology domain) linked to an Fc monomer linked to a Fab fragment via peptide linkers (see FIGS. 1-4). The scfps dimerized to form the BFPs. Sequences used for BFPs are described below.

### Example No. 2. Octet Binding Assay

To evaluate binding of the bispecific binding molecules disclosed herein, an Octet QK equipped with Ni-NTA biosensor tips and 10×kinetics buffer were used (ForteBio, Menlo Park, Calif.). For this series of bispecific binding proteins, His-tagged PD-L1-his was made in-house, CD40-Fc protein was purchased from Sino-Biological (Beijing, China) and biotinylated in the lab. All binding assays were performed at 25° C.

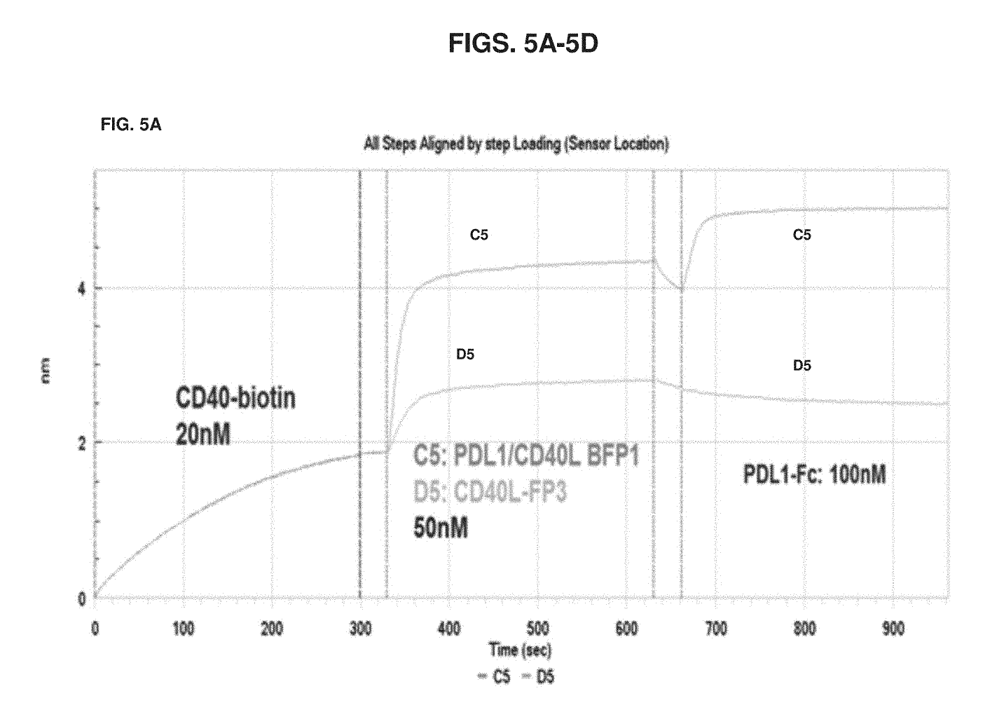

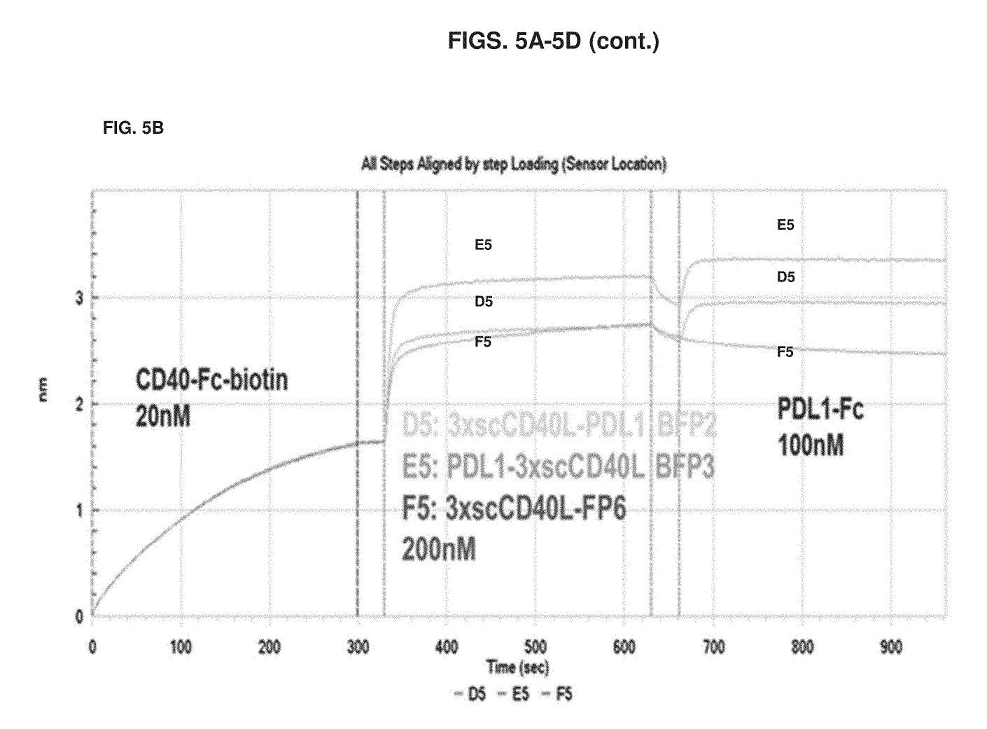

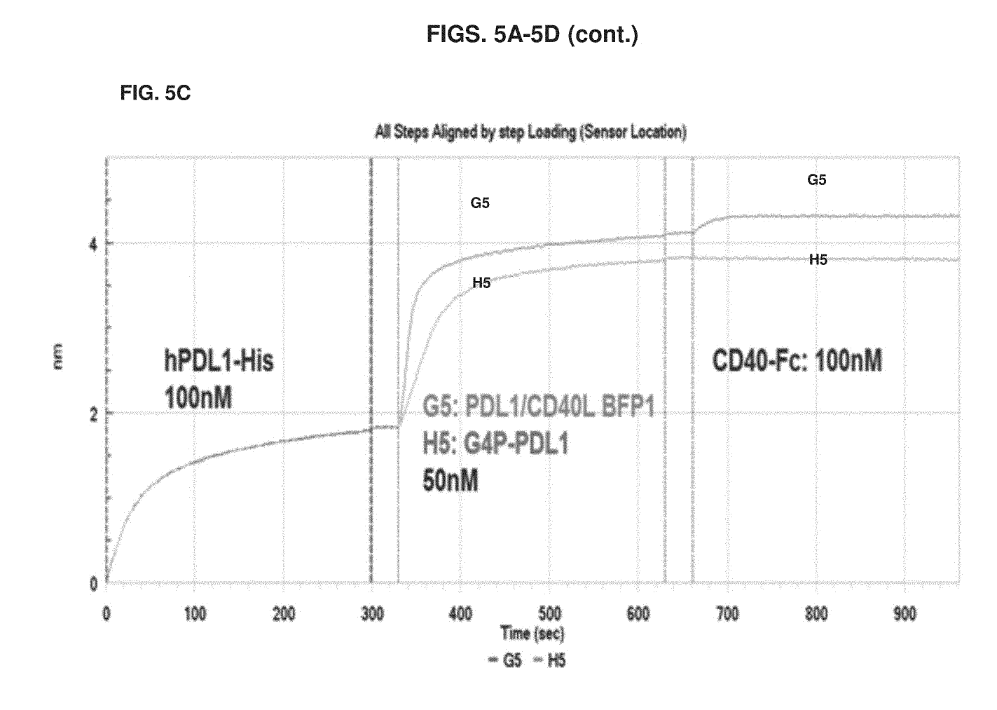

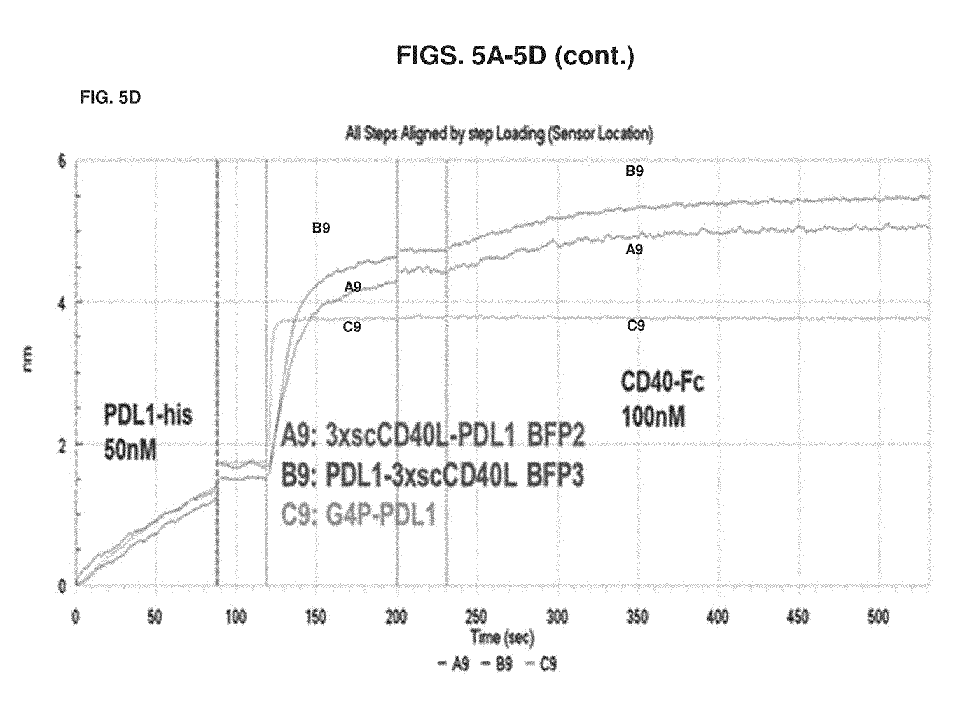

Sample plates were agitated at 1000 rpm prior to analysis. 1×kinetic buffer was applied to the Streptavidin and Ni-NTA biosensor tips for 10 mins prior to us. The 1×kinetic buffer also served as the running buffer for baseline determination and as the dilution buffer for antigens and bispecific antibodies. Streptavidin or Ni-NTA biosensor tips were dipped into 20 nM CD40-biotin (FIGS. 5A and 5B) or his-tagged PD-L1 (FIGS. 5C and 5D) for antigen capture for 5 min and rinse in kinetic buffer for 30 seconds. The antigen coated biosensor tips were each dipped into 10 μg/m1 bispecific antibodies for 5 minutes and rinsed, then moved into a column of wells containing 100 nM PD-L1 Fc antigen (FIGS. 5A and 5B) or 100 nM CD40-Fc (FIGS. 5C and 5D) for 5 minutes. In this assay, when BFP molecules have been saturated the first antigen (CD40 or PD-L1), the second antigen (PD-L1 or CD40) was injected and, as expected, a second binding signal was observed. This observation was reproducible when the antigen injection order was reversed, indicating BFP molecules can bind to two targets simultaneously.

### Example No. 3. Assessment of Cell Surface Antigen Binding

Flow cytometry was used to evaluate cell surface antigen binding by BFPs.

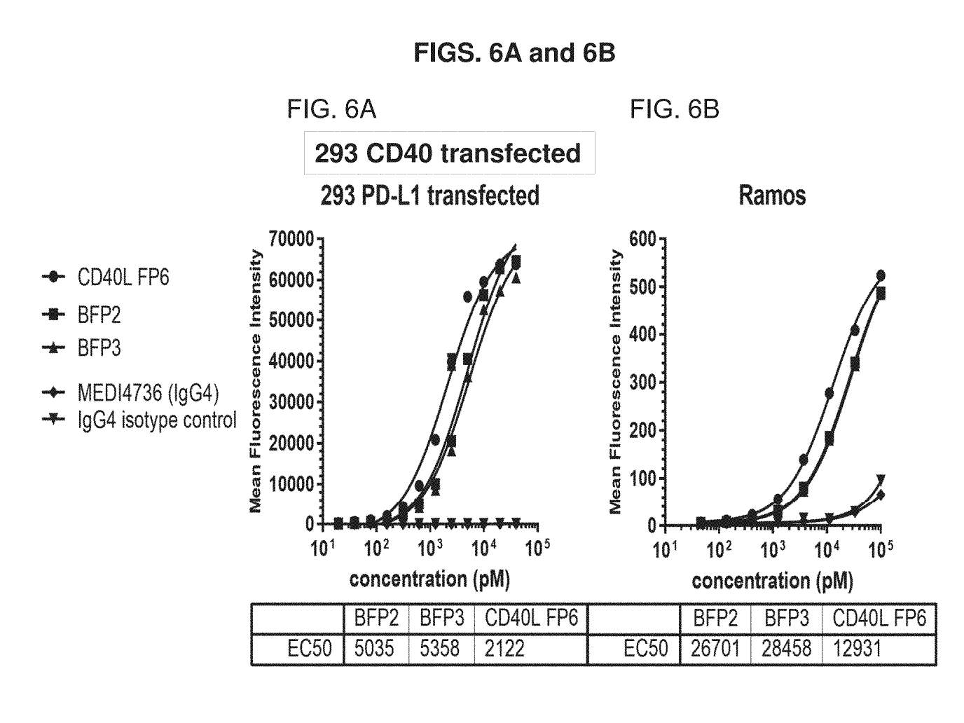

Anti-PD-L1+CD40L FPs BFP2 and BFP3 (MEDI7526; see Table 1), CD40L FP6 (MEDI5083), and anti-PDL1 (MEDI4736), all in human IgG4 isotype, were conjugated to Alexa Fluor 647 using Alexa Fluor 647 Monoclonal Antibody Labeling Kit (Thermo Fisher). An IgG4 isotype control antibody was also conjugated following the same protocol. All resulting conjugated antibodies had similar dye to antibody ratios. In the binding assay, Alexa 647 conjugated antibodies were serially diluted in FACS buffer (PBS plus 3% fetal calf serum), resulting in final concentrations between 40 nM to 19.532 pM and mixed with 10,000 CD40 transfected HEK293 cells (FIG. 6A) or Ramos human B cells (FIG. 6B). Both CD40 transfected 293 and Ramos cells express CD40 but not PD-L1. After incubation at 4° C. for one hour, cells were spun down and free antibodies in the supernatant were removed. Cells with bound antibodies were washed and run through flow cytometry. Fluorescence signals were detected and recorded by the cytometry machine and mean fluorescence intensive of Alexa 647 on target cells were determined using Flowjo® software. The graphs of FIGS. 6A and 6B show that BFP2 and BFP3 have similar binding to CD40 as the parental MEDI5083; whereas, anti-PD-L1 (MED4736) and isotype control antibody did not bind.

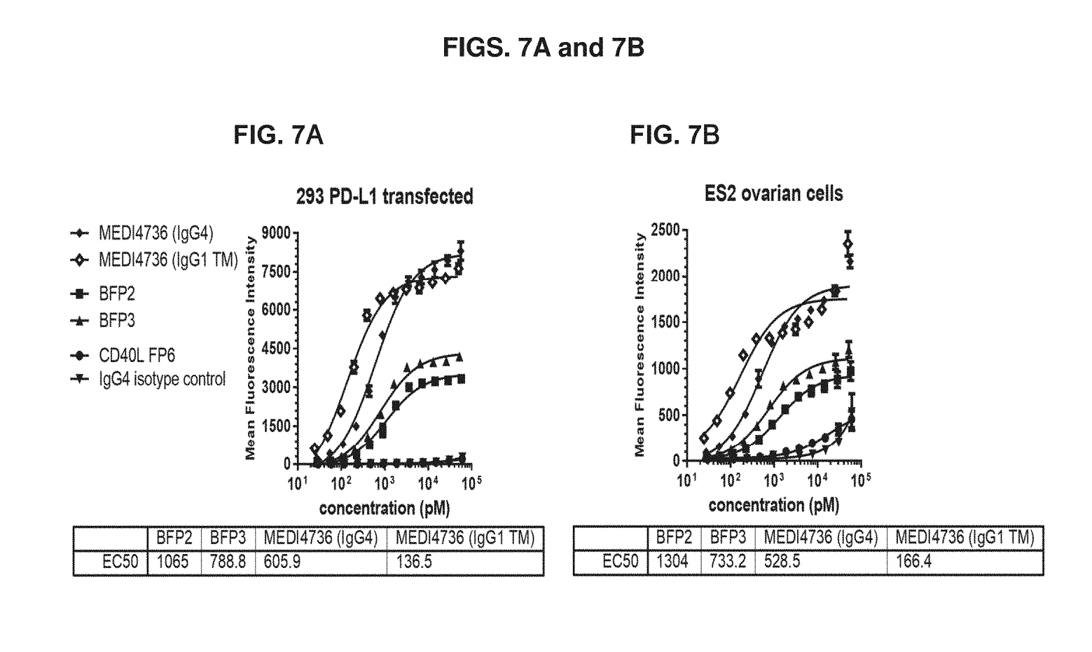

Next, binding to human PD-L1 on PD-L1 transfected HEK293 cells and an ES2 human ovarian cancer cell line were evaluated. Both cell lines expressed PD-L1 but not CD40. Alexa 647 conjugated antibodies were diluted in FACS buffer and mixed with 10,000 PD-L1 transfected HEK293 cells. After incubation at 4° C. for one hour, cells were spun down and free antibodies in the supernatant were removed. Cells with bound antibodies were washed and run through flow cytometry. Fluorescence signals were detected and recorded by the cytometry machine and mean fluorescence intensive of Alexa 647 on target cells were determined using Flowjo® software. The graphs of FIGS. 7A and 7B show that both BFP2 and BFP3 bind to cell surface PD-L1, though with lower maximum binding and higher EC₅₀ than MEDI4736 with IgG4 or IgG1 TM isotypes. CD40L FP and isotype control antibody did not bind to PD-L1 in this assay, as expected.

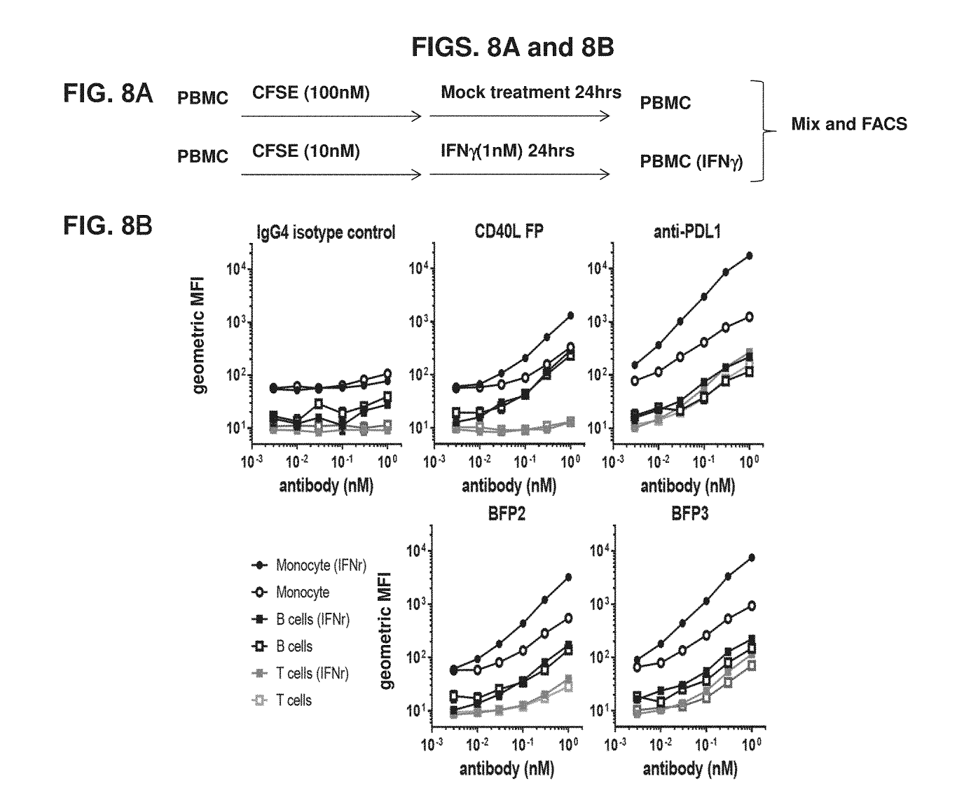

Anti-PD-L1+CD40L FP, BFP2 and 3 were evaluated for binding to human PBMCs. Since expression of CD40 and PD-L1 is increased on PBMCs under inflammatory conditions, we evaluated binding on pooled naïve and IFN-γ stimulated PBMCs. In this study, PBMCs were isolated from a healthy donor and labeled with either high (100 nM) or low (10 nM) amounts of carboxyfluorescein diacetate succinimidyl ester (CFSE). PBMCs with 100 nM CFSE were cultured without treatment for 24 hours and PBMCs labeled with 10 nM CFSE were cultured under the same conditions but stimulated with 1 nM human IFN-γ overnight to upregulate CD40 and PD-L1 expression. Thus, cells with or without IFN-γ treatment can be differentiated based on different levels of CFSE signals. All PBMCs with high and low CFSE labeling were mixed on the next day and stained with anti-CD19 for B cells, CD3 for T cells, and CD14 for monocytes. Binding of anti-PD-L1-CD40L FP BFP molecules to PBMC subsets was revealed by flow cytometry in comparison with CD40L FP (MEDI5083) and anti-PDL1 (MEDI4736 IgG1 TM). As shown in FIG. 8, both formats of BFP proteins exhibited similar binding activity and potency. IFN-γ-treated monocytes bind to most BFP molecules, followed by naïve monocytes and T and B cells. These results also indicate that on PBMCs, BFP molecules have similar binding profile as anti-PDL1 and CD40L FP (MEDI5083).

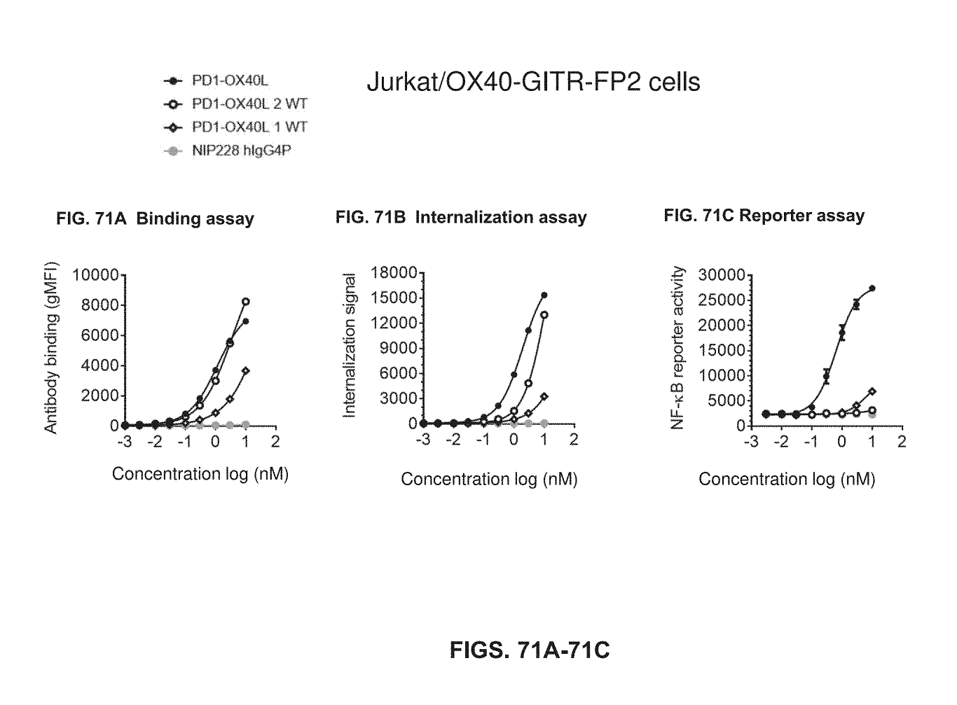

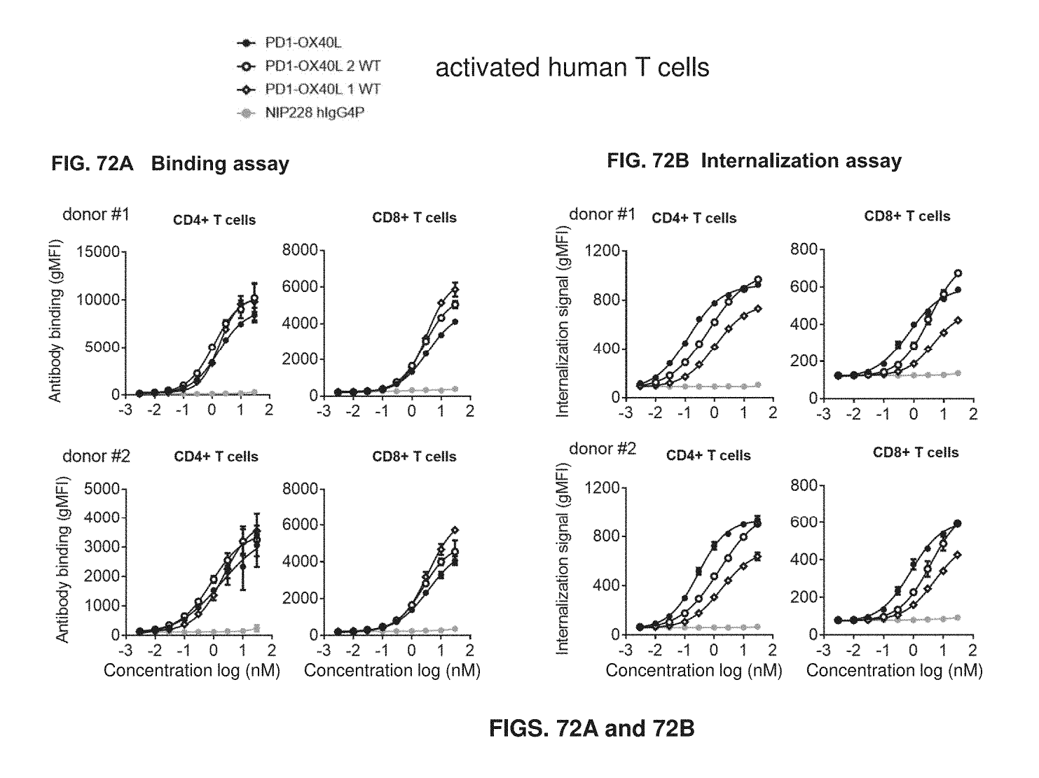

PD1-OX40L, PD1-OX40L 2WT and PD1-OX40L 1WT all in human IgG4 isotype, were conjugated to Alexa Fluor 647 using Alexa Fluor 647 Monoclonal Antibody Labeling Kit (Thermo Fisher). Binding to Jurkat/OX40-GITR-FP2 cells (FIG. 71A) and to activated human primary T cells (FIG. 72) were tested using the protocol as mentioned above.

### Example No. 4. Anti-PDL1-CD40L FP, BFP2 and 3 Have the Ability to Stimulate CD40 Pathway and Block PD1-PDL1 Interaction

### NF-κB Activation Pathway is Triggered via CD40 Activation.

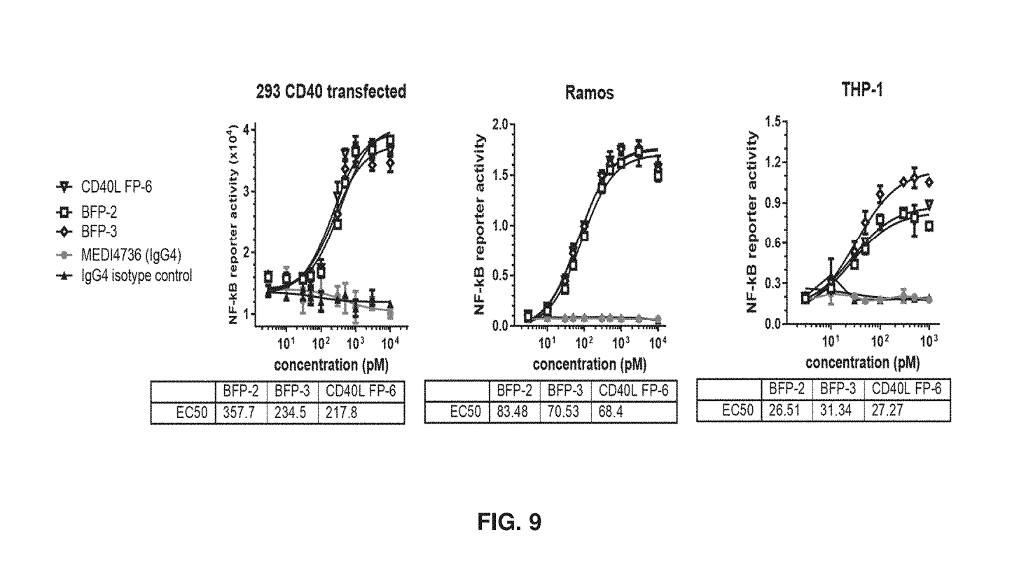

HuCD40/HEK293/NF-κB cells (clone 3) were maintained in DMEM (GIBCO) plus 10% Heat-inactivated FBS (HI-FBS; GIBCO) and 1% Pen/Strep (GIBCO). On day 1, cells were harvested and resuspended in DMEM with 2% HI FBS at 5×10⁵/mL. One hundred microliters per well of cells were seeded in a BD Biocoat Poly-D-lysine 96 well black/clear microtiter plate (Cat#356640). Cells were placed in a 37° C. incubator for 24 hours. After incubation, the medium was aspirated from the plate. One hundred microliters of 1×test material was carefully added to each well and care was taken to minimize detachment of the cells. The cells were returned to the 37° C. incubator for 24 hours. Luciferase reagent (Bright-Glo® Luciferase Assay Substrate; Promega) was prepared, allowed to equilibrate to room temperature, and added (100 μL) to each well. The cells and reagent were mixed well to ensure complete cell lysis, and immediately read on a SpectraMax M5 plate reader. FIG. 9 demonstrates that BFP molecules activated NF-κB signals on multiple cell types, including CD40 transfected 293 cells (A), Ramos cells (b) and THP-1 cells (C).

### Ramos-Blue Bioactivity Assay Protocol

Ramos-Blue NF-κB/AP-1 reporter cells (Invivogen) were maintained in IMDM GlutaMAX® (GIBCO) plus 10% HI-FBS (GIBCO), 1% Pen/Strep (GIBCO) and Zeocin (100 μg/mL; InvivoGen) media. The cells are non-adherent, and cultures were initiated at 5×10⁵ cells/mL and kept below 3×10⁶ cells/mL. On the day before the experiment, cells were split into IMDM GlutaMAX plus 10% HI-FBS and pen/strep (Zeocin-free) media. Cells were harvested, adjusted to 1×10⁶ cells/mL, and added (180 μL) to the wells of a flat-bottomed 96-well plate (Corning). Twenty microliters of 10×test material in Zeocin-free media was added to each well, and the cells were placed in a 37° C. incubator for 24 hours. QUANTI-Blue reagent (one pouch dissolved in 100 mL sterile water; Invivogen) was prepared and added to a flat-bottomed 96-well plate at 160 μL/well. Supernatant from the Ramos-Blue cells (40 μL) was the added to the wells containing QUANTI-Blue. Plates were placed in a 37° C. incubator for up to 1 hour, and read on a SpectraMax M5 spectrophotometer at 655 nm.

### THP1-Blue Bioactivity Assay Protocol

THP1-blue NF-κB reporter cells (Invivogen) were maintained in RPMI1640 (GIBCO) plus 10% HI-FBS (GIBCO), 1% Pen Strep (GIBCO) and blasticidin (10 μg/mL; InvivoGen) media. The cells are non-adherent, and cultures were initiated at 7×10⁵ cells/mL and kept below 2×10⁶cells/mL. On the day before experiment, cells were split into RPMI1640 plus 10% HI-FBS and pen/strep (blasticidin-free) media. Cells were harvested, adjusted to 1×10⁶cells/mL, and added (180 μL) to the wells of a flat-bottomed 96-well plate (Corning). Twenty microliters of 10-fold test material (already serially diluted) in blasticidin-free media were added to each well, and the cells were placed in a 37° C. incubator for 24 hours. QUANTI-Blue reagent (one pouch dissolved in 100 mL sterile water; Invivogen) was prepared and added to a flat-bottomed 96-well plate at 160 μL/well. Supernatant from the THP1-Blue cells (40 μL) was the added to the wells containing QUANTI-Blue. Plates were placed in a 37° C. incubator for 6 hours, and read on a SpectraMax M5 spectrophotometer at 655 nm. Results are shown in FIG. 9.

### Example No. 5. Attenuation of PD-L1-Mediated Inhibition

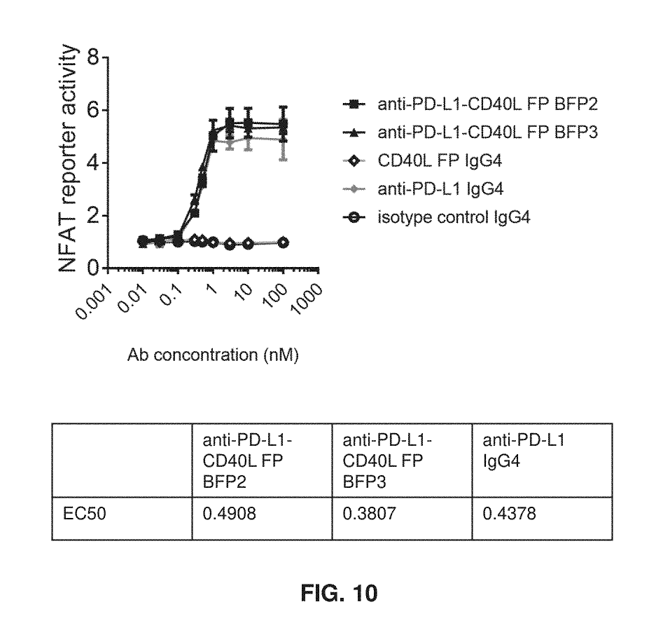

In this example, anti-PD-L1-CD40L FP BFP molecules were examined to determine whether they were biologically equivalent to anti-PD-L1 in the PD-1PD-L1 blockade bioassay (Promega). First, one vial of CHO PD-L1 cells was thawed and resuspended in 14.5 ml of Ham's F12 media with 10% FBS. Cells were added at 100 μL per well to 96-well, white bottom assay plates. The plates were incubated at 37° C. incubator overnight (16-20 hours). The plates were taken out from the incubator the next day and media were carefully removed. Forty (40) μL of test material (2×) in assay buffer (RPMI1640 with 1% FBS) was added to each well of the plates. Next, one vial of Jurkat PD1 effector cells was thawed and resuspended in 5.9 mL of assay buffer. Forty (40) μL, of Jurkat PD1 cells was then added to each well of the plates. The plates were placed at 37° C. incubator for 6 hours. Luciferase reagent (Bio-Glo® Luciferase Assay Substrate; Promega) was prepared, allowed to equilibrate to room temperature, and added (80 μL) to each well. The plates were placed at room temperature for 5 min followed by immediate read on a SpectraMax® M5 plate reader. MEDI7526 inhibited PD-L1 function, resulting in a dose-dependent increased of NFAT activity in this assay. The EC₅₀ of MEDI7526 is comparable to the MEDI4736 IgG4 molecule. FIG. 10 demonstrates anti-PD-L1-CD40L FP BFP attenuated PD-L1-mediated inhibitory function.

### Example No. 6. BFP Coactivation Assay

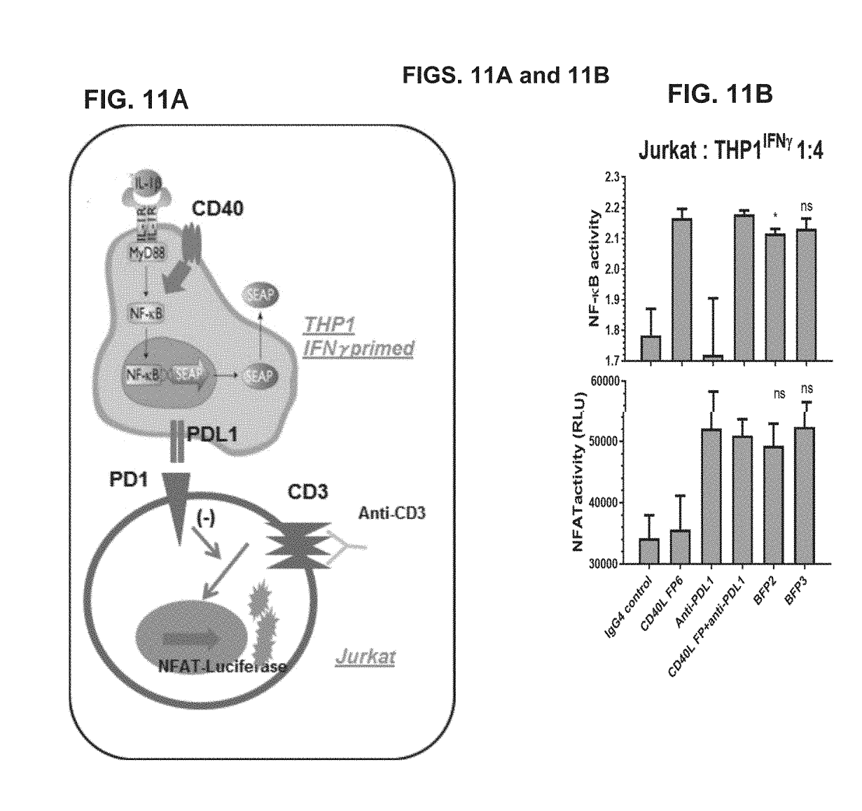

BFP molecule function was further evaluated in a robust reporter-based THP-1 monocyte and Jurkat T cell co-activation assay. In this assay, THP-1 cells transfected with NF-κB-SEAP reporter genes (Invivogen) were seed at 400,000 per well and stimulated with IFN-γ overnight to upregulate CD40 and PD-L1 expression on these cells. IFN-γ stimulation does not induce NF-κB activation on THP-1 cells. An assay conceptual schematic is shown in FIG. 11A.

The night before assay day, a white 96-well plate was coated with anti-human CD3 antibody (Biolegend). On the assay day, the THP-1 cells were washed and mixed with 100,000 per well Jurkat cells transfected with NFAT-luciferase reporter (Promega) and serial diluted test reagents were added. The assay plate was incubated at 37° C. for 6 hours. Cells were then spun down and 40 μL, of culture medium were transferred from each well to the corresponding well of a new 96-well plate and were stored at −80° C.

To determine SEAP activity, which is present in culture medium, plates with frozen cell culture medium were thawed at room temperature and mixed with QUANTI-Blue solution at 37° C. After 15 minutes incubation, assay SEAP activity was measured with a microplate reader. To measure NF-κB activity, cells were mixed with 1×lysis reagent and cell lysates were mixed with 80 μL Bio-Glo luciferase assay reagent (Promega). Plates were incubated for 5 minutes at ambient temperature and luminescence intensities were measured in a plate reader SpectraMax® M5. As shown in FIG. 11B, anti-PDL1-CD40L BFP molecules activated NF-κB in THP1 cells and increased NFAT activity in Jurkat T cells, demonstrating that BFP molecules can perform multiple functions on mixed immune cells.

### Example No. 7. MEDI7526 Activates Primary Human Cells and Induces Production of Cytokines

In this study, the ability of MEDI7526 to induce cytokine production in primary cells was examined.

### Staphylococcal Enterotoxin B (SEB) Assay Protocol

Reagents used in the SEB assay protocol to determine the effect of the BFP molecules on IL-2 immune response include: Leukocyte cones (NHSBT code NC24; from Addenbrookes Hospital); 50 ml Falcon tubes (BD 352070); Ficoll-Paque PLUS (GE Healthcare 17-1440-02); Anti-CD3 (clone OKT3; 1 mg/ml; eBioscience; cat no:16-0037-85); Ammonium chloride solution (Stemcell Technologies 07850); Staphylococcal enterotoxin B (SEB; Sigma, S-4881) stock solutions at 1 mg/mL stored at −20° C.; Culture media (all from Life Technologies): RPMI1640 with GlutaMax™ (61870) supplemented with 10% v/v heat inactivated FCS (90005M) and 100 U/mL penicillin+100 μg/mL streptomycin (15140-122); V-bottomed plate (Greiner BioOne 651201); 96-well flat-bottom plates (Corning Costar 7107).

Reagents for the IL-2 DELFIA ELISA include: FLUONUNC Maxisorp ELISA plates (Nunc 437958); Europium-labelled streptavidin, SA-Eu (Perkin-Elmer 1244-360); DELFIA® assay buffer (Perkin-Elmer, #4002-0010); DELFIA® enhancement solution (Perkin-Elmer 4001-0010); at RT prior to use; Assay diluent: DELFIA wash buffer (0.05% Tween-20, 20 mM Tris, 150 mM NaCl; pH 7.2-7.4) supplemented with 0.1% BSA, sterile filtered; Milk powder (Marvel; Premier Foods); Sample Diluent (RPMI1640+10% FCS+1% Penicillin/Streptomycin as above); PBS (ThermoFisher 14190235); PBS-Tween (0.01% Tween-20 in PBS); Human IL-2 ELISA kit (Duoset DY202, R&D Systems); Biotek plate washer (EL406) with automated plate loader (Biostack).

### General Assay Protocol

PBMCs were isolated from human blood leukocyte cones (NHS Blood and Transplant Service code NC24) using density gradient centrifugation (Ficoll-Paque PLUS; GE Healthcare), then red blood cells were lysed in ammonium chloride solution (Stemcell Technologies). Anti-human CD3 (clone OKT3 at 0.5 μg/mL in PBS; eBioscience) was coated in flat-bottomed 96 well plates (Corning Costar 7107) for 2 hours at 37° C. Then, 2×10⁵ cells were added per well of the PBMCs in culture media (RPMI1640-GlutaMax supplemented with 10% v/v heat inactivated bovine serum and 100 U/100 μg per mL Streptomycin/Penicillin (respectively) (Life Technologies). PBMCs were further stimulated by addition of SEB final concentration 1 μg/mL, and candidate BFP molecules were serial diluted added to the final tested concentrations. Following 3 days culture at 37° C. and 5% CO₂ supernatants were removed from cells, and IL-2 secretion was determined using a commercial ELISA according to manufacturer's instructions (R&D Systems).

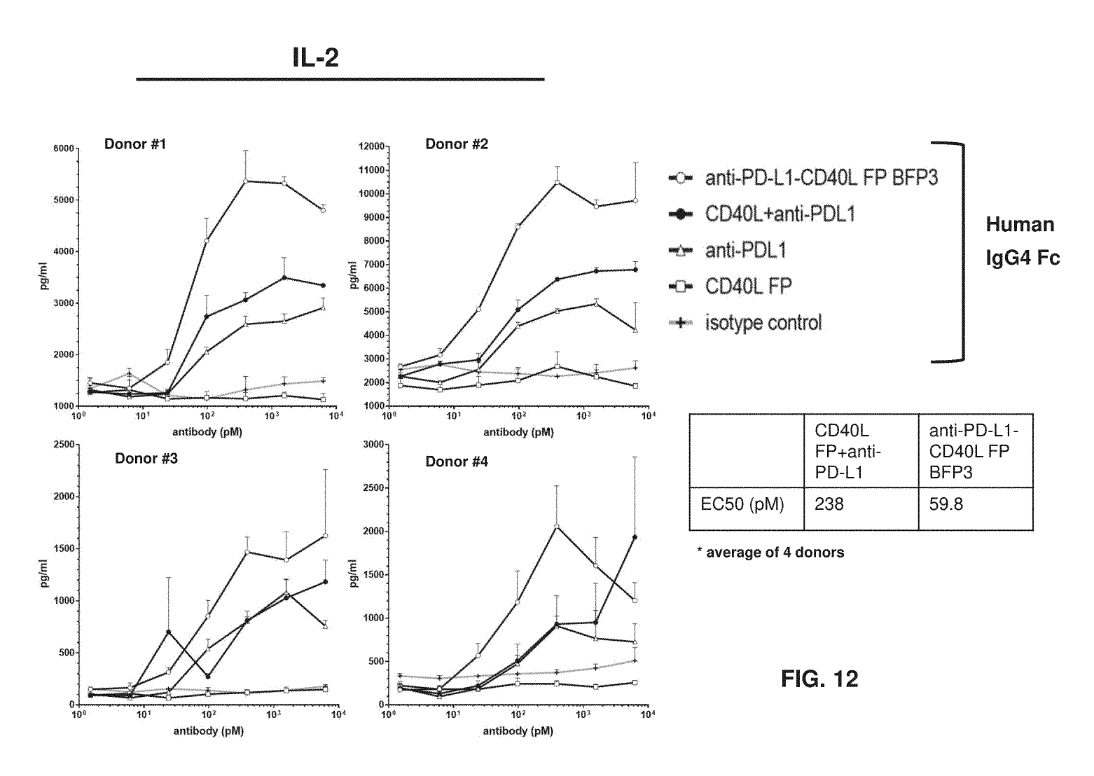

The results shown in FIG. 12 demonstrate that MEDI7526 (BFP3) induces the highest level of IL-2 production in the SEB assay with an EC50 of 59.8 pM.

### Example No. 8. MEDI7526 Activates Primary Human Cells and Induces Production of Cytokines

In this study, the abilities of MEDI3387 and MEDI5771 to induce cytokine production in primary cells were examined.

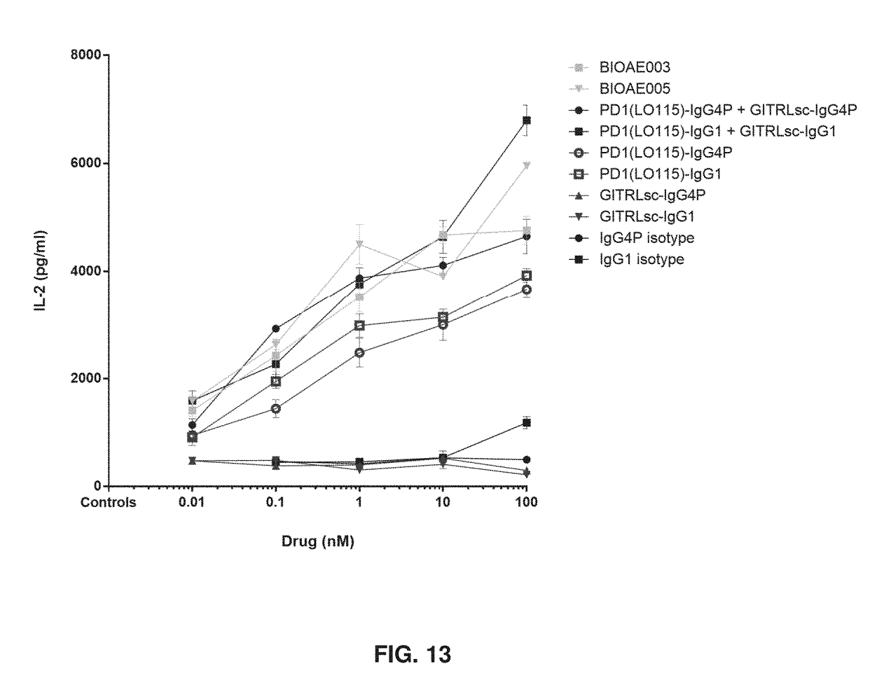

PBMC were prepared from Leukocyte cones (supplied by the NHSBT, Addenbrooke's Hospital) using Ficoll-Paque PLUS (GE Healthcare 17-1440-02), following the manufacturer's recommended protocol. PBMC were resuspended in culture media (RPMI1640 with glutamax [Gibco] supplemented with 10% heat inactivated FCS [Life Technologies 90005M] and 1% penicillin/streptomycin) and transferred to 96 well flat-bottomed tissue culture plates (Corning Costar 7107) previously coated with anti-human CD3 antibody (coating was performed by adding 225 μL of PBS containing 0.5 μg/mL of OKT3 [Ebioscience cat: 16-0037-85] to each well and incubating for 2 hours at 37° C. prior to use). Reactions had a final volume of 225 μL per well, contained 2E5 cells, and were supplemented with Staphylococcal Enterotoxin B (at a concentration of 0.1 μg/mL) together with test drug or control mAbs. Reactions were incubated for 72 hours at 37° C., 5% CO₂, after which supernatants were removed and tested subsequently for IL-2 release by ELISA. The results are shown in FIG. 13.

### Example No. 9. MEDI7526 MLR Assay

Induction of IFN-γ and IL-12 production in macrophages by MEDI7526 was assessed using an MLR assay.

### General Assay Protocol

Culture monocyte-derived M1 macrophage: monocytes were isolated from one donor using EasySep™ Human CD14 Positive Selection Kit (STEMCELL) and M1 macrophage were generated using CellXVivo Human M1 Macrophage Differentiation Kit (R&D systems). In this assay, 40 million monocytes were split into 2 T75 flasks. Half of the media was removed from each flask and replaced at days 3 and 6 with fresh media supplemented with GM-CSF. On day 6, differentiated macrophages were harvested using StemPro™ Accutase™ Cell Dissociation Reagent (Invitrogen), centrifuge cells at 1500 rpm for 5 minutes. Next, the supernatant was removed and the cells were in complete RPMI 1640 media at 0.125 million/ml. Next, 80 μL/well of macrophages were added to 96-well U bottom plates and 20 μL of testing antibodies (10-fold of final concentration) were added per well. Next, 100 μL/well of isolated total T cells from another donor (1 million/mL) were added to 96-well U bottom plates. The plates were incubated at 37° C. in a CO₂ incubator for 5 days. The supernatant was harvested and levels of cytokines in the supernatant were measured with human Th1/Th2 10-plex kit (Meso Scale Discovery).

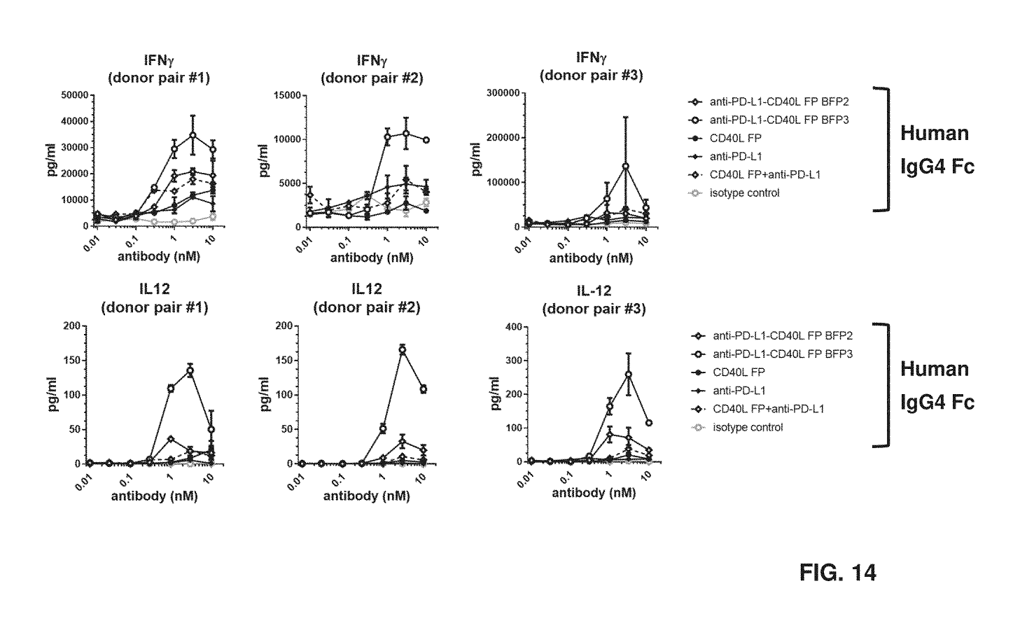

FIG. 14 shows that MEDI7526 (BFP3) induced production of IFN-γ on 3 macrophage-T cell MLR reactions.

### Example No. 10. Mixed Leukocyte Reaction (MLR) Assay Protocol (Fresh Blood)

The MLR cell-based assay described in Example No. 9 was also used to provide in vitro correlation of T cell function in response to BFP molecules disclosed herein.

### General Assay Protocol

Monocytes were isolated from PBMCs of one donor and T cells from another donor. Both monocyte and T cells were suspended in complete RPMI medium at 1:1 ratio and incubated with testing reagents. The plates were incubated at 37° C. for 5 days. One the last day, plates were centrifuged at 300 g for 5 minutes and the supernatant was harvested. Cytokines in the supernatant were measured with human Th1/Th2 10-plex kit (Meso Scale Discovery).

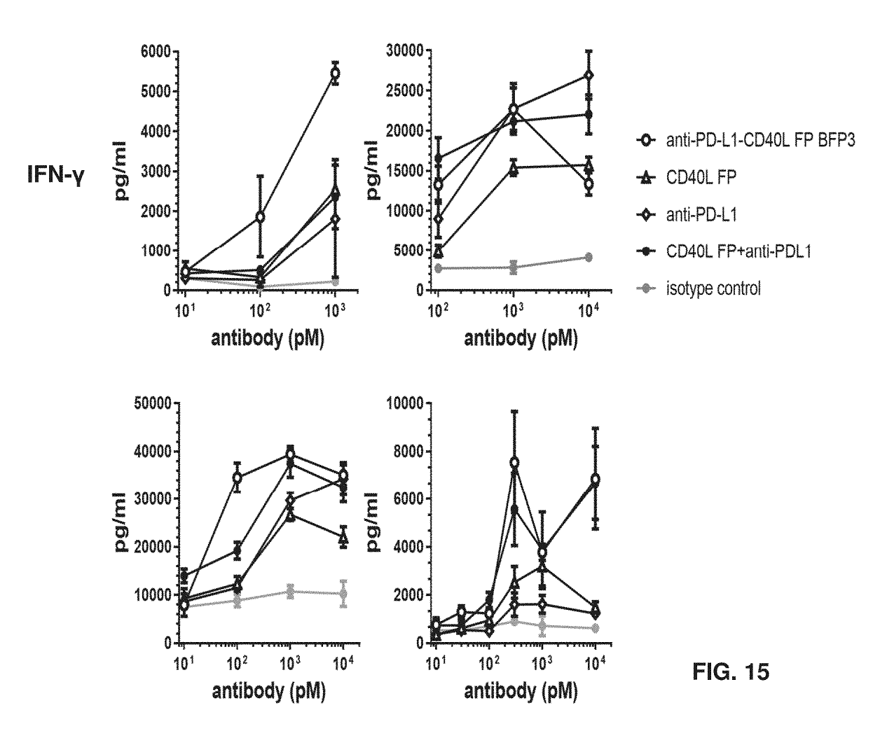

FIG. 15 shows that MEDI7526 (BFP3) induced production of IFN-γ on 4 pairs of monocyte-T cells MLR reaction, suggesting that MEDI7526 can boost T-cell mediated immune responses.

### Example No. 11. CMV Recall Assay

A cytomegalovirus (CMV) antigen recall assay was used to evaluate the potential immune response induced by certain of the immunotherapeutic molecules described herein. The induction of IFN-γ secretion in response to recombinant human CMV pp65 protein (CMV pp65) by CD8+ T-cells in a CMV positive donor is termed an immune memory recall response. Certain cancers can enhance this suppression through expression of these receptors leading to the interest in therapeutically interfering with these immune checkpoints. There is a potential benefit to antibody combination, the use of bispecific reagents and altering the Fc-component type.

In this experiment, HLA-A02 typed PBMC from known CMV-positive donors were exposed to the HLA-A02 restricted CMV pp65 peptide (495-503) in the presence of BFP molecules. After 4 days, IFN-γ secretion was determined by MSD. General reagents used are shown in Table 2.

TABLE 2
General reagents for CMV Recall Assay

| Description | Manufacturer | Cat# | Notes |
|---|---|---|---|
| XVIVO-15 media | LONZA | 04-418Q | |
| CMV pp65 - HLA-A02 | AnaSpec | AS-28328 | Dissolve 1 mg in |
| peptide | | | 1 mL DMSO |
| 96-well plates - T/C- | Falcon | 353077 | U-bottom wells |
| treated | | | |
| Human IFN-γ kit | MSD | | |

### CMV Recall-Assay Protocol (Astarte Biologics/MedImmune Hybrid):

CMV-positive human PBMC were thawed, washed with XVIVO-15, counted, and adjusted to 4×10e6 cells/mL in XVIVO-15. Two microliters of CMV pp65 HLA-A02 peptide were added per mL cell suspension and mixed well. Next, 100 μL of PBMC plus peptide were to wells (4×10e5 cells/well). To each well, 100 μL antibody (2×) were added, and the plates were placed in a 37° C. incubator. On day 4, the supernatant (100 μL) was harvested from each well and frozen at −30° C. for the subsequent cytokine assay (MSD).

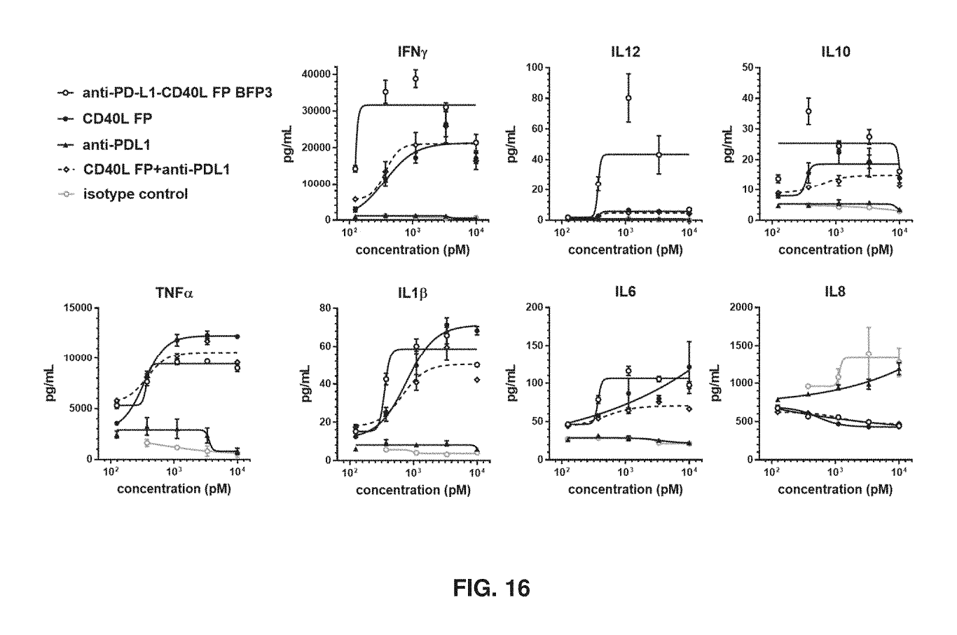

FIG. 16 demonstrates that MEDI7526 induced higher levels of IFN-γ and IL-12 production in the CMV recall assay than other test samples. For IFN-γ: BFP3 had an EC50 of ˜104.3, CD40L FP6 (MEDI5083) had an EC50 of 359.4, CD40L+anti-PD-L1 had an EC50 of 367.1.

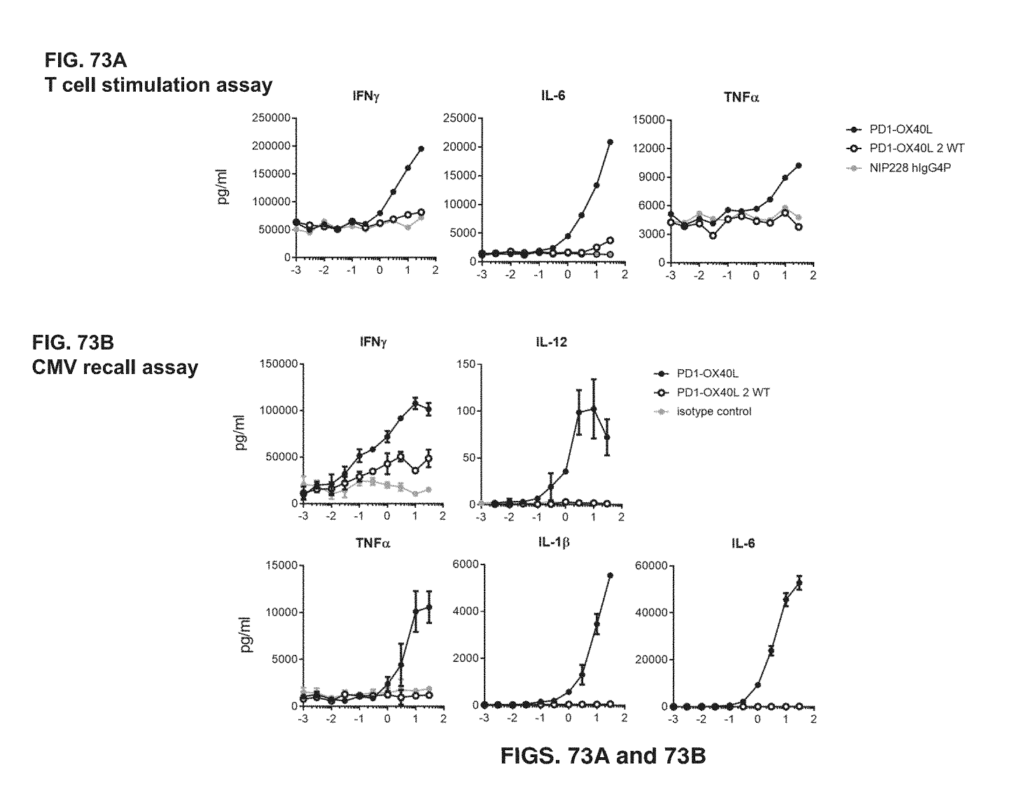

FIG. 73B shows that PD1-OX40L BFP induced higher levels of IFN-γ, IL-12, TNFα, IL-1β(3 and IL-6 production in the CMV recall assay than PD1-OX40L 2WT BFP, indicating residual F180 is critical for OX40 agonist function in this assay.

### Example No. 12. BFP Molecules Trigger Internalization and Degradation of PD1 OR PDL1

In this example, the hypothesis that BFP3 induces CD40 and PD-L1 internalization and destabilizes membrane PD-L1 was tested.

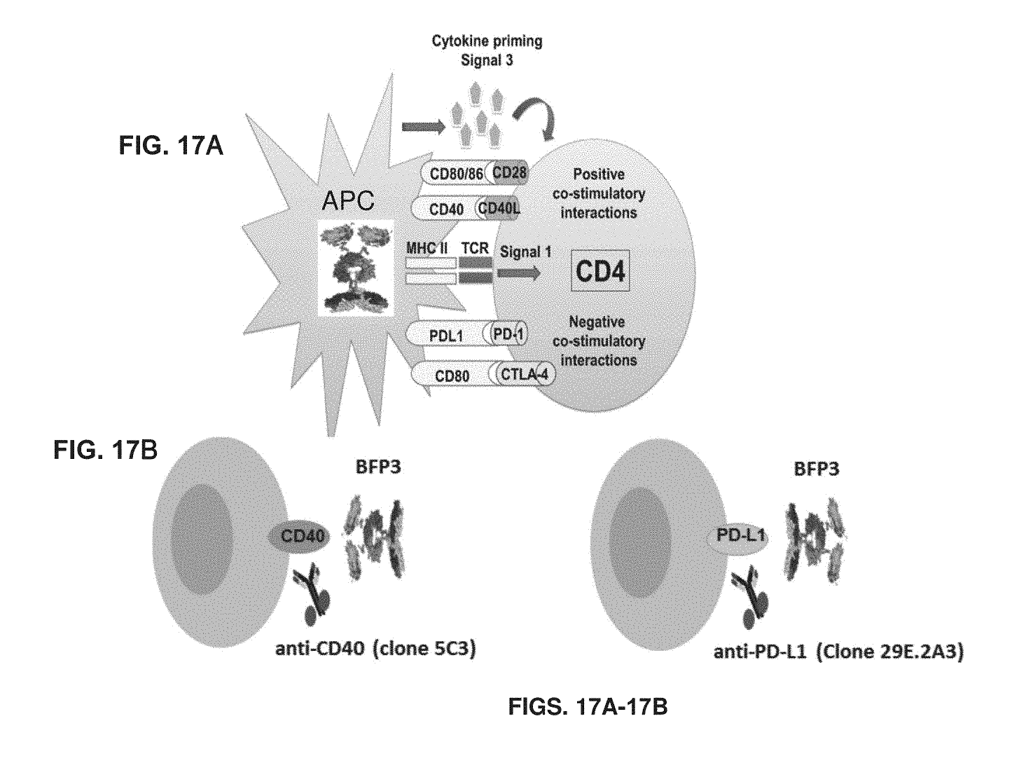

MEDI7526 BFP3 binds to human CD40 and PD-L1. It was hypothesized that BFP3 could induce CD40 and PD-L1 internalization and subsequently trigger PD-L1 protein degradation. To enable quantitative measurements of internalization of CD40 and PD-L1 upon MEDI7526 treatment, a panel of anti-CD40 and PD-L1 antibodies were screened and anti-CD40 clone 5C3 and anti-PD-L1 clone 29E.2A3 were identified as non-competing antibodies. Namely, the anti-CD40 clone 5C3 does not compete with MEDI7526 to bind CD40, and the anti-PD-L1 clone 29E.2A3 does not compete with MEDI7526 to bind PD-L1. The non-competing antibodies were taken advantage of to evaluate surface expression of CD40 and PD-L1 after MEDI7526 treatment. FIG. 17 depicts a flow cytometry based method for detecting internalization of CD40 and PD-L1 from the cell surface.

### MDA-MB-231 Cells

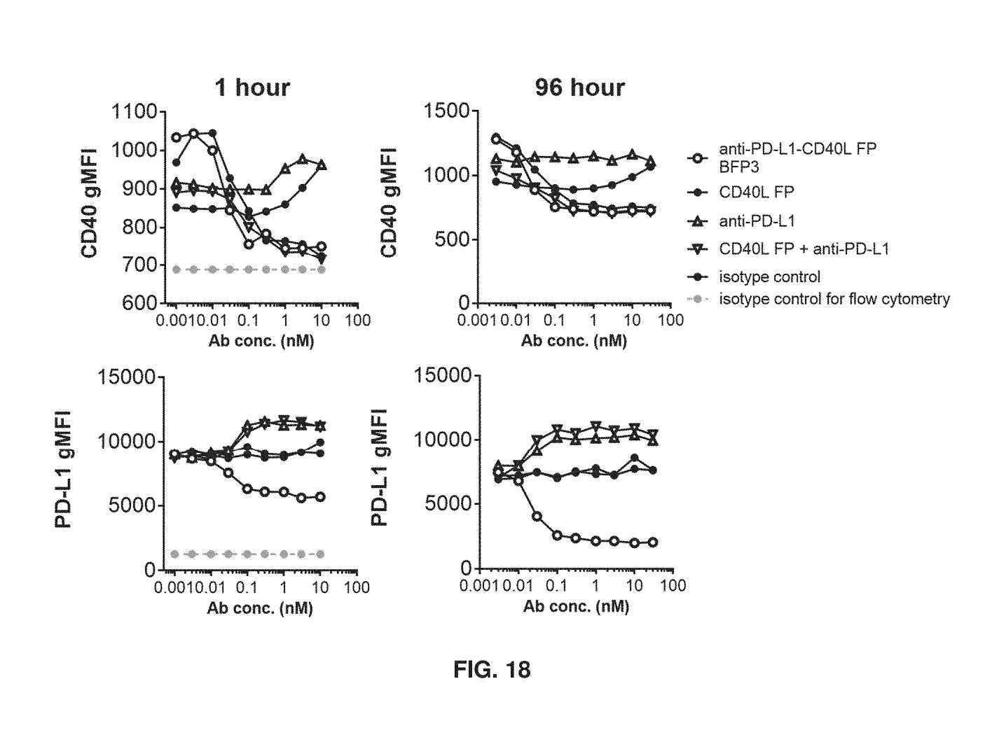

MDA-MB-231 is a human breast adenocarcinoma cell and constitutively expresses both CD40 and PD-L1. MDA-MB-231 cells were mixed with titrated amount of testing material and incubated at 37° C. for either 1 hour or 96 hours. After incubation, free antibodies were removed by washing and cells were stained with fluorochrome-conjugated anti-CD40 (clone 5C3) and anti-PD-L1 (clone number 29E.2A3), both from BioLegend. Then, the cells with bound antibodies were subjected to flow cytometry analysis. Geometric mean fluorescence intensity was calculated using Flowjo® and plotted in the graph. The results are seen in FIG. 18.

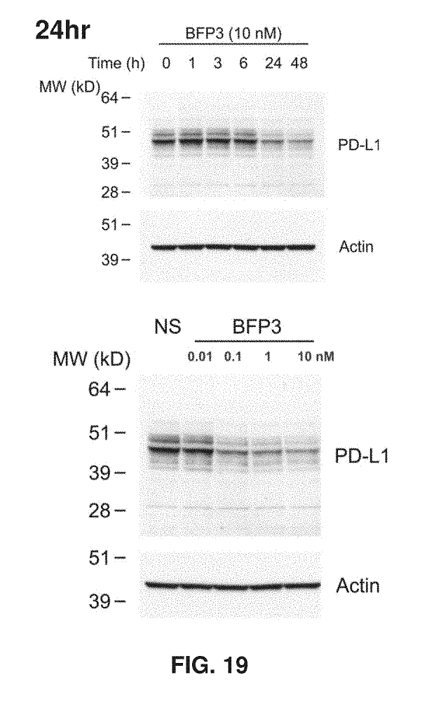

Next, MDA-MB-231 cells were plated 0.5 million per well in a 6-well plate with RPMI1640 medium and treated with indicated conditions. After 24 hours, cells were lysed in 300 μL/well of RIPA buffer (Millipore) with protease inhibitors followed by incubation at 4° C. for 1 hr with rotation. The amount of protein in the lysate was determined by BCA analysis (Pierce) and presence of PD-L1 protein was detected by Western blot using anti-PD-L1 clone (E1L3N) from Cell Signaling (see FIG. 19).

The results seen in FIGS. 18 and 19 demonstrate that MEDI7526 (BFP3) treatment not only down-regulated CD40 and PD-L1 surface expression on the MDA-MB-231 cells but also decreased total PD-L1 protein content in MDA-MB-231 cells.

### THP-1 Cells

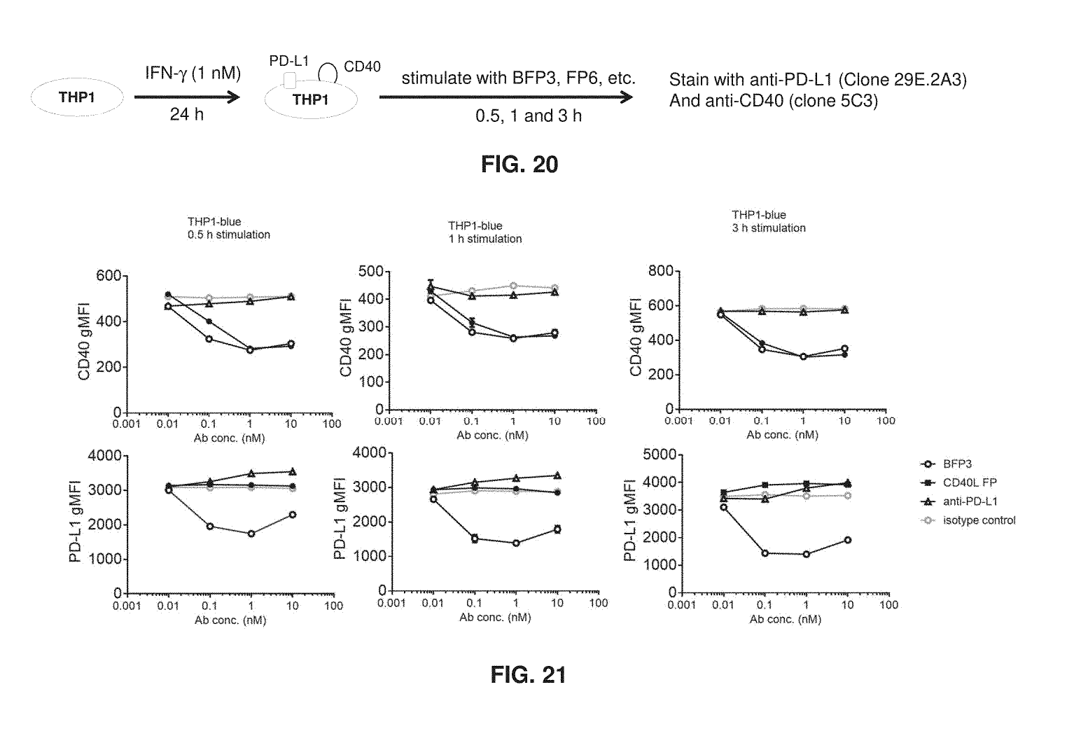

THP-1 cells are a human leukemic monocyte cell line. THP-1 cells express very low amount of CD40 and PD-L1 but upregulate expression of CD40 and PD-L1 post IFN-γ treatment. In the assay schematic shown in FIG. 20, THP-1 cells were stimulated with IFN-γ for 24 hours and mixed with titrated amounts of testing material and incubated at 37° C. for 0.5 to 3 hours. After incubation, free antibodies were removed by washing and cells were stained with fluorochrome-conjugated anti-CD40 (clone 5C3) and anti-PD-L1 (clone number 29E.2A3). Then, the cells with bound antibodies were subject to flow cytometry analysis. Geometric mean fluorescence intensity was calculated by Flowjo® and plotted in FIG. 21, which demonstrates that MEDI7526 induced rapid down-regulation of CD40 and PD-L1 from cell surface of THP1 cells between 0.5˜3 hours.

### IFN-γ-Treated THP-1 Cells

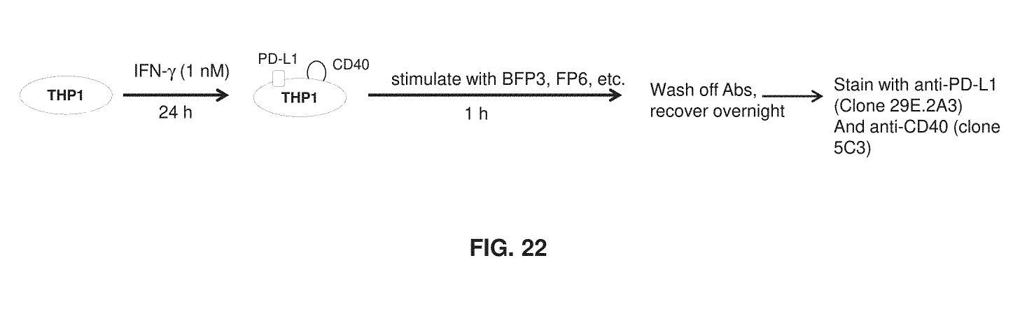

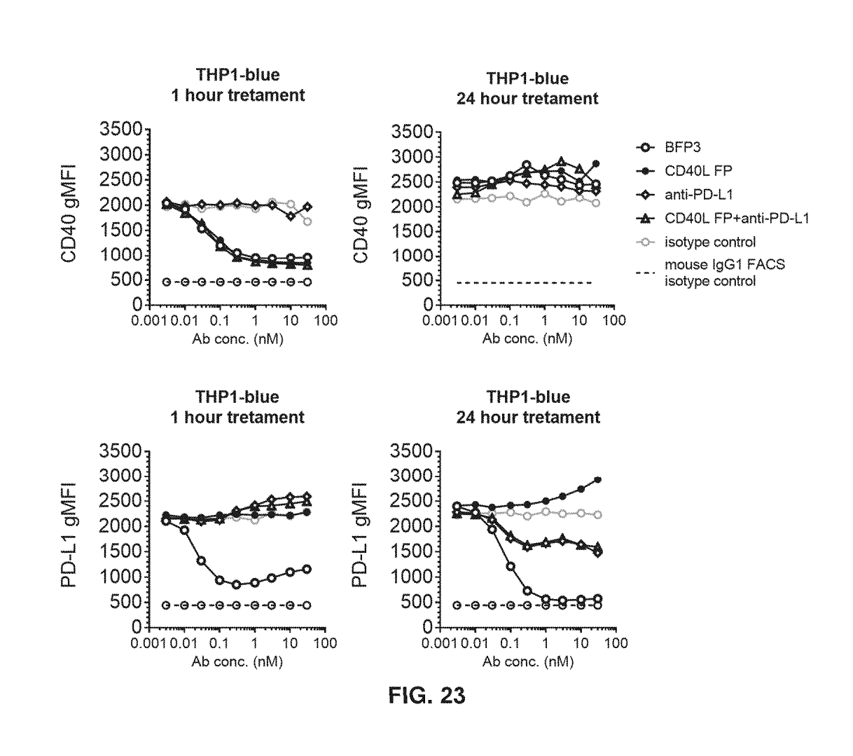

Following the study schematic in FIG. 22, THP-1 cells were stimulated with IFN-γ for 24 hours and mixed with titrated amounts of testing material and incubate at 37° C. for 1 hour. After incubation, free antibodies were removed by washing and cells were stained with fluorochrome-conjugated anti-CD40 (clone 5C3) and anti-PD-L1 (clone number 29E.2A3). Then, the cells with bound antibodies were subject to flow cytometry analysis. Geometric mean fluorescence intensity (gMFI) was calculated by Flowjo® and plotted in the graphs shown in FIG. 23, which demonstrate that CD40 expression is quickly recovered at 24 hour post treatment but PD-L1 cell surface expression remains low, indicating separate recovery pathways for internalized CD40 and PD-L1.

### Example No. 13. Human Primary Cell Assay

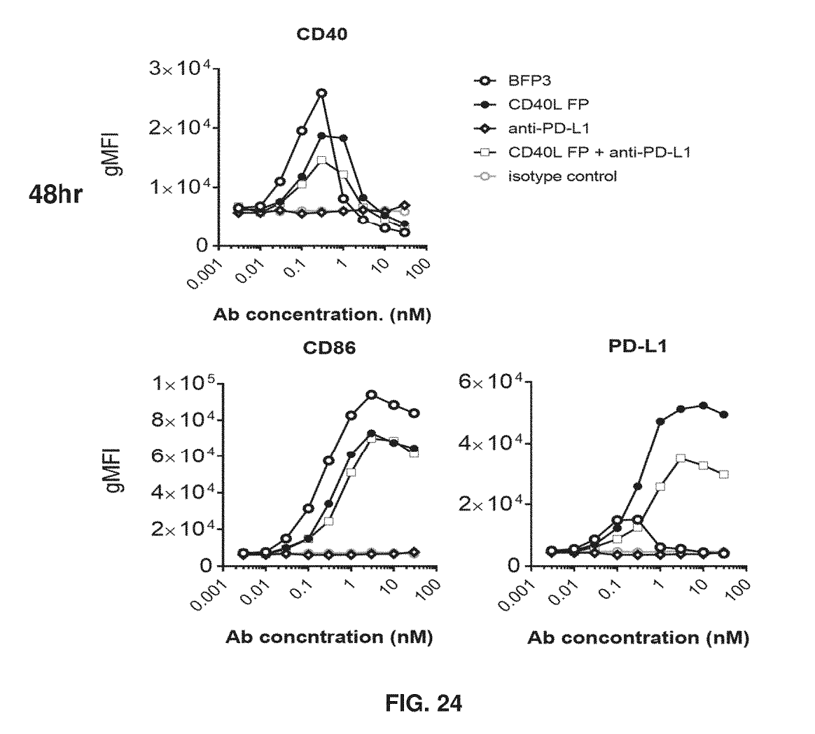

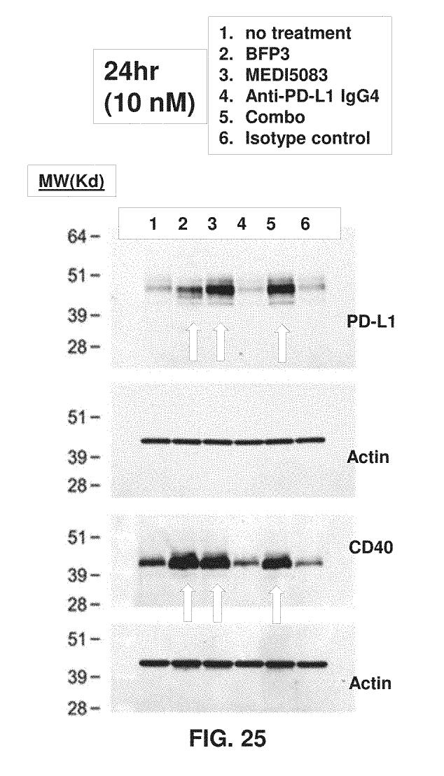

In this study, monocytes were isolated from healthy donor PBMCs using EasySep™ Human CD14 Positive Selection Kit (STEMCELL) and were differentiated into dendritic cells using a CellXVivo Human Monocyte-derived DC Differentiation Kit (R&D, Cat # CDK004) according to the manufacturer's protocol. Cells were resuspended in human DC differentiation medium including GM-CSF and IL-4 at 1 million cells/mL, and a total 20 million cells were seeded in a T75 flask. Half of the media was replaced on day 3 and 5 with fresh human DC differentiation medium. At day 7, immature DC were harvested and stimulated with increasing doses of test material. Surface expression of CD40, CD86 and PD-L1 were determined by flow cytometry after stimulation for 24 hours (see FIG. 24). In FIG. 25, protein levels of CD40 and PD-L1 in DCs stimulated with 10 nM of test material for 24 hours were measured by immunoblotting. These results show that MEDI7526 (BFP3) caused downregulation of PD-L1 on human primary cells.

FIG. 24 demonstrates that MEDI7526, like its parental CD40L FP induced upregulation of CD40 at low doses but down-regulated CD40 at high doses. It also upregulated CD86 expression on monocyte-derived dendritic cells. But PD-L1 protein levels stayed low on MEDI7526-treated cells.

### PBMC Assay

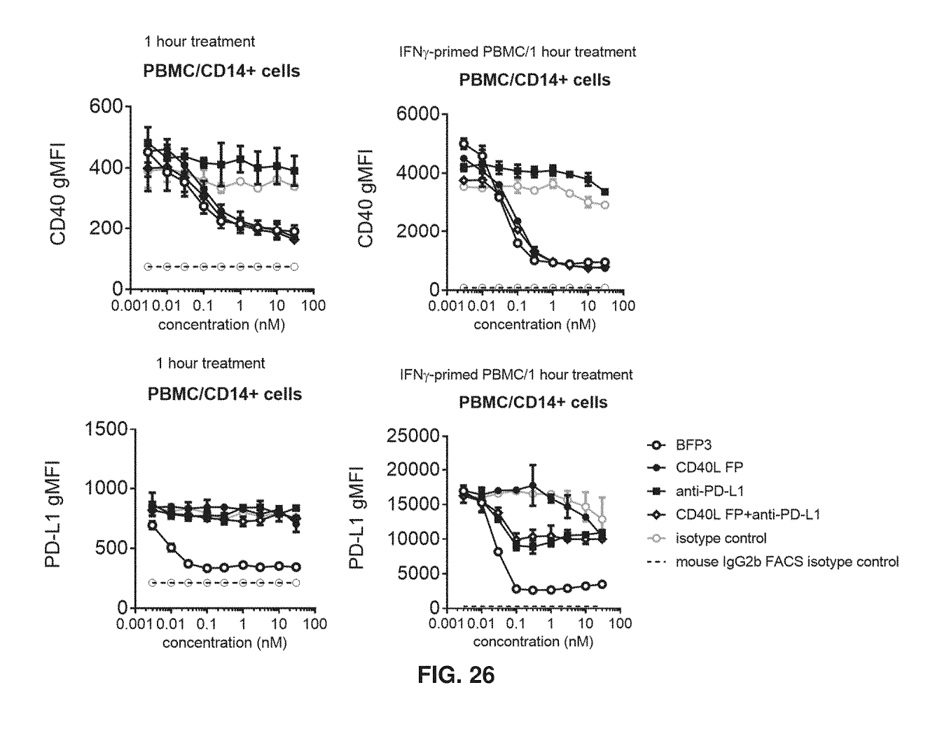

In this study, PBMCs from healthy donors were stimulated with IFN-γ and reacted with test reagents for one hour. Surface expression levels of CD40, CD86, and PD-L1 were studied with flow cytometry. FIG. 26 demonstrates that MEDI7526 induces down-regulation of CD40 and PD-L1 from cell surface of monocytes. These results show that MEDI7526 (BFP3) caused downregulation of PD-L1 on freshly isolated human primary cells.

### Example No. 14. Murine Surrogate MEDI7526 Efficacy Against PD-L1 in Renca Cells

Renca is a murine kidney renal adenocarcinoma cell line that constitutively expresses both CD40 and PD-L1. In this study, Renca cells were cultured at 0.5 million/well in a 6-well plate on the first day. On the second day of culture, Renca cells were treated with indicated reagents at the concentration of 10 nM for 24 hrs. On the third of culture day, treated Renca cells were lysed in RIPA buffer with protease inhibitors, and cell lysates were analyzed by Western blot. Antibodies used for Western blot are listed in Table 3 below.

TABLE 3
Antibodies

| Antibody | Vendor | Cat# | Dilution | Host |
|---|---|---|---|---|
| anti-mPD-L1 | R&D systems | AF1019 | 1:500 | goat |
| anti-CD40 | Abcam | ab13545 | 1:500 | rabbit |
| anti-actin | Sigma | A2066-.2ML | 1:2000 | rabbit |
| Peroxidase | Jackson | 711-035-152 | 1:4000 | Donkey |
| AffiniPure Donkey | Immunoresearch | | | |
| Anti-Rabbit | | | | |
| IgG (H + L) | | | | |

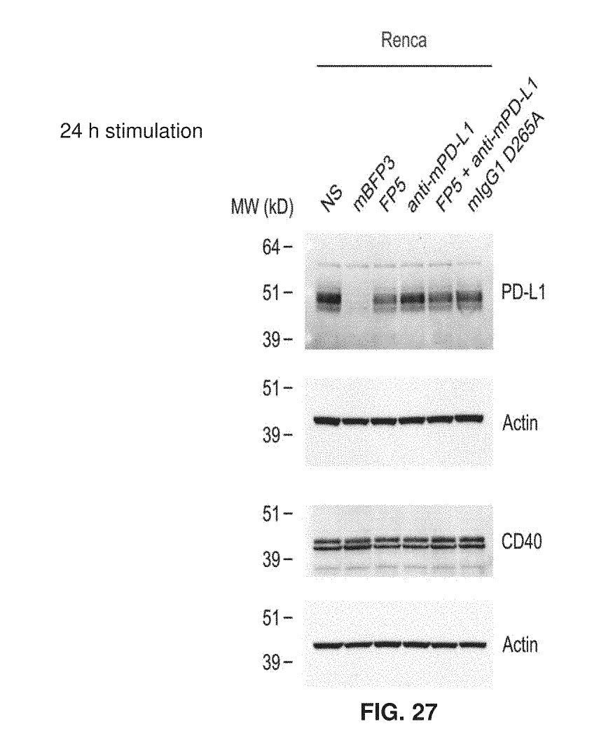

FIG. 27 demonstrates that a murine surrogate of MEDI7526 (mBFP3) induced degradation of PD-L1 in Renca cells. This result demonstrates that a murine surrogate of MEDI7526 has similar function in downregulation of PD-L1 and CD40 expression on murine cells.

### Example No. 15. Engagement of BFP Molecules and FC Receptor can Enhance Myeloid Cell Activation

In this example, myeloid cell activation by BFP molecules was examined.

ES2 cells, which express PD-L1, were seeded at 30,000 cells/well in a flat bottom 96-well plate, and THP1 cells were stimulated with IFN-γ for 24 hours. On the 2^{nd} day, equal amounts of ES2 cells and THP-1 cells were mixed and titrated testing materials (BPF1, BPF2, MEDI7526, FP6 (MEDI5083), and MEDI4736; see Table 1) were added. The plates were incubated at 37° C. for 24 hrs. QUANTI-Blue™ was prepared following the instructions on the pouch. Next, 160 μL of QUANTI-Blue solution were mixed with 40 μL of supernatant per well of a flat-bottom 96-well plate. The plate was incubated at 37° C. for 3 hours, and SEAP levels were determined using a spectrophotometer at 655 nm.

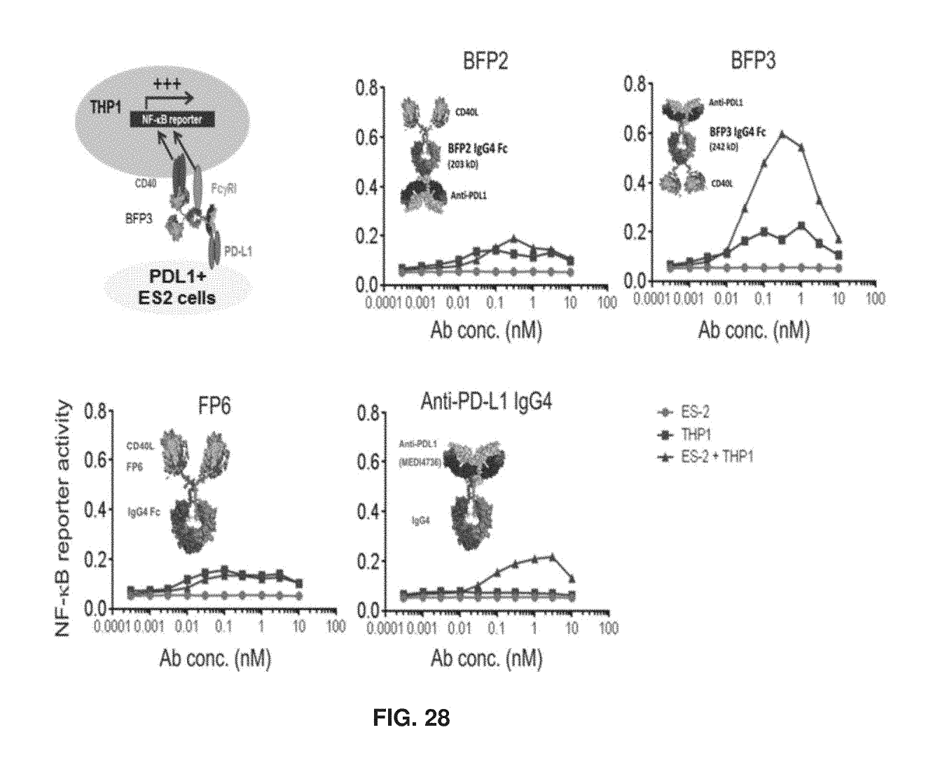

FIG. 28 shows that cross-linking through PD-L1 on tumor cells can enhance MEDI7526 BFP3 activities. The orientation of the BFP format appears to impact function, as it was observed that the BFP2 format did not have enhanced activity. This result based on a 2-cell system demonstrates that cross-linking through PD-L1 on tumor cells can augment MEDI7526 function in stimulating NF-κB activity in THP-1 cells.

### Example No. 16. Role of FCγRI in PD-L1 Cross-Linking

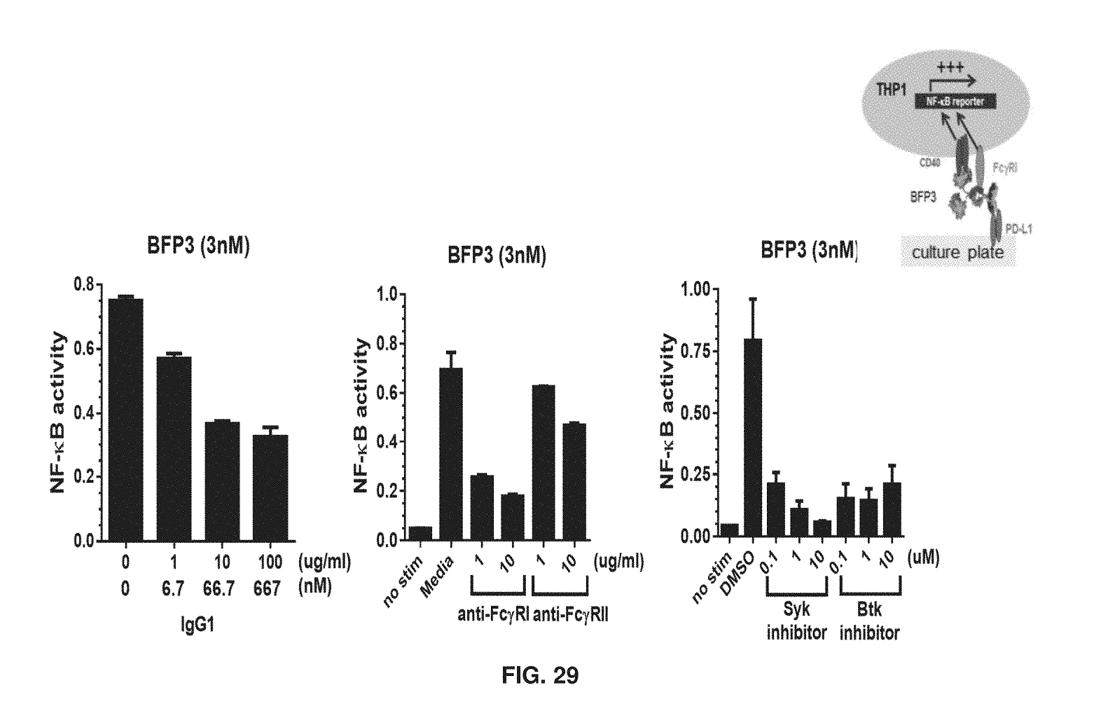

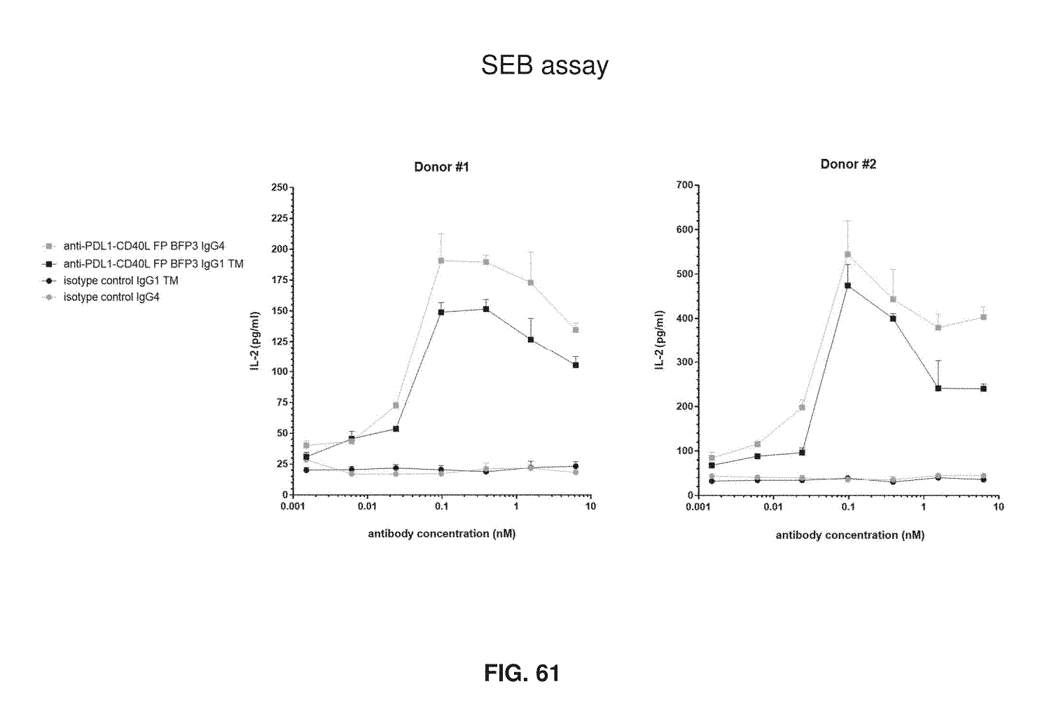

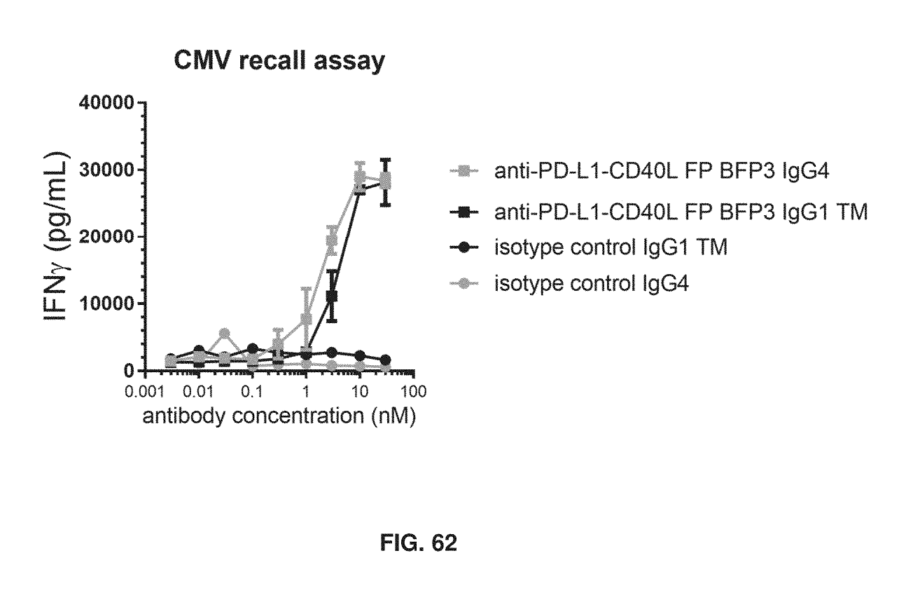

In this example, the role of the high affinity Fc receptor (FcγRI) in PD-L1 crosslinking was examined. Biocoat 96-well plates (Corning) were coated with 50 μL/well of PD-L1-His (2 μg/ml, R&D systems) overnight at 4° C. On the 2nd day, THP1 cells (0.2 million/well) were added to the plates. Soluble IgG1 (made in house) was added, which competes with IgG4 in binding to Fcγ receptors. Other inhibitors including blocking antibodies against FcγRI/FcγRII (Biolegend) and inhibitors for Syk and Btk (Sellechem) for 1 hour followed by stimulation with test materials (3 nM) for 24 hours. On the 3rd day, plates were centrifuged at 1500 rpm for 5 min. Forty (40) μL of cell culture supernatant were mixed with 160 μL of freshly prepared QUANTI-Blue™ reagents (Invivogen) followed by incubation at 37° C. for 1 hour. SEAP levels were determined by a SpectraMax® M5 spectrophotometer at 655 nm. FIG. 29 demonstrates that enhanced signals mediated by PD-L1 cross-linking are through FcrRI and can be inhibited by soluble IgG and inhibitors for Syk and Btk. However, Fc engagement is not required for BFP3 function (see FIGS. 61 and 62). These results suggest that augmented activity mediated by BFP3 is through FcγRI engagement.

### Example No. 17. A Murine Surrogate of MEDI7526 Demonstrates Robust Anti-Tumor Activity in Vivo and That It is Tolerable in Mouse Tumor Model

To study the effect of MEDI7526 in vivo in mice, a murine surrogate of MEDI7526, mMEDI7526 was constructed. In parallel with MEDI7625, mMEDI7526 comprises, from N- to C- terminus, a F(ab)2 anti-murine PD-L1, a murine IgG1 Fc with D265A mutation, and two single chain fusion proteins of 3×murine CD40L subunits connected via peptide linkers.

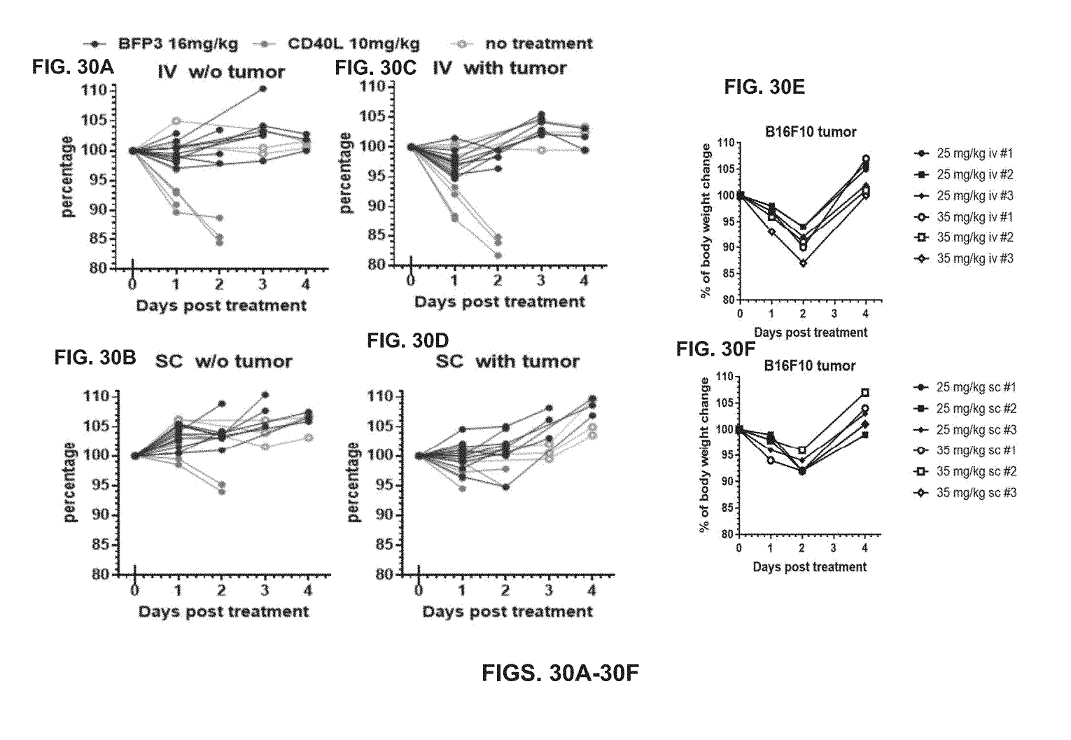

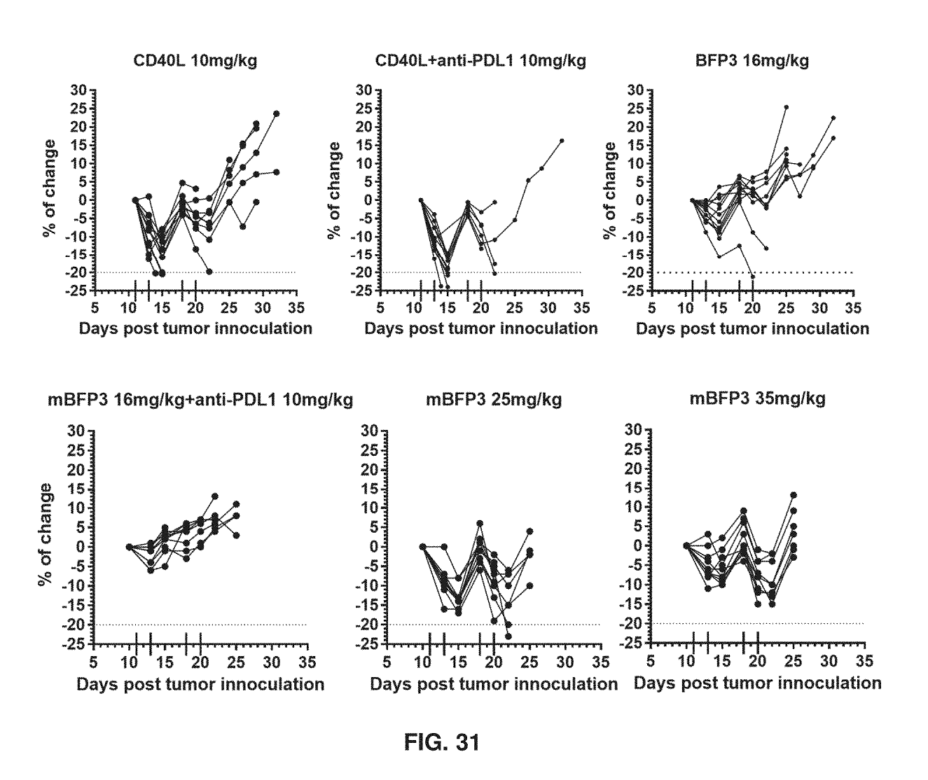

Murine surrogate of MEDI7526 (mMEDI7526) was tested first in the C57B1/6 female mice to study its safety profile (FIGS. 30A and 30B). Naïve mice received single intravenous (iv; FIG. 30A) or subcutaneous (sc; FIG. 30B) treatments with either mCD40L at 10 mg/kg or mMEDI7526 at an equivalent molar concentration of 16 mg/kg. An untreated group of mice was used as a control. Body weights were monitored before and every day post treatment and were converted to percentage of the baseline body weight for individual mice (see FIG. 31). Compared to control, mCD40L treatment, either iv or sc, led to loss of body weight. In comparison, mMEDI7526 treatment at 16 mg/kg did not cause significant loss of body weight.

In a separate experiment, C57B1/6 female mice were implanted with B16F10 tumor cells and mice with tumor sizes >100 mm³ were selected for the following studies. Selected mice received a single iv or sc treatment of mCD40L (10 mg/kg) and mMEDI7526 (16 mg/kg) as shown in FIGS. 30C and 30D, or with high doses of mMEDI7526 (25 or 35 mg/kg, FIGS. 30E and 30F, respectively). Body weights were monitored every day before and after treatment and percentages of baseline body weight were calculated and compared between treatment groups to the no treatment group. The results demonstrate that treatment of mCD40L led to more severe loss of body weight post treatment and indicates that mMEDI7526 is more tolerable than mMEDI5083 in naïve or tumor bearing mice.

Next, the safety profile of mMEDI7526 was evaluated in a multiple-dose study on the B16F10 murine tumor model. Mice were implanted with B16F10 tumors on day 1 and mice with tumor sizes>100mm3 were randomized on day 11, followed by the treatment on days 11, 13, 19 and 21, as indicated in FIG. 31. Mice with more than 20% loss of body weight were considered under severe stress and removed from the study. Immediately after 2^{nd} dosing on day 13, 2 mice in the mCD40L group and 4 in the CD40L+anti-PDL1 group had >20% loss of body weight. In comparison, none of mMEDI7526 dosed mice showed >20% loss of body weight after 2^{nd} dosing. These data further indicate that mMEDI7526 is more tolerable than mCD40L alone or in combination with anti-PDL1.

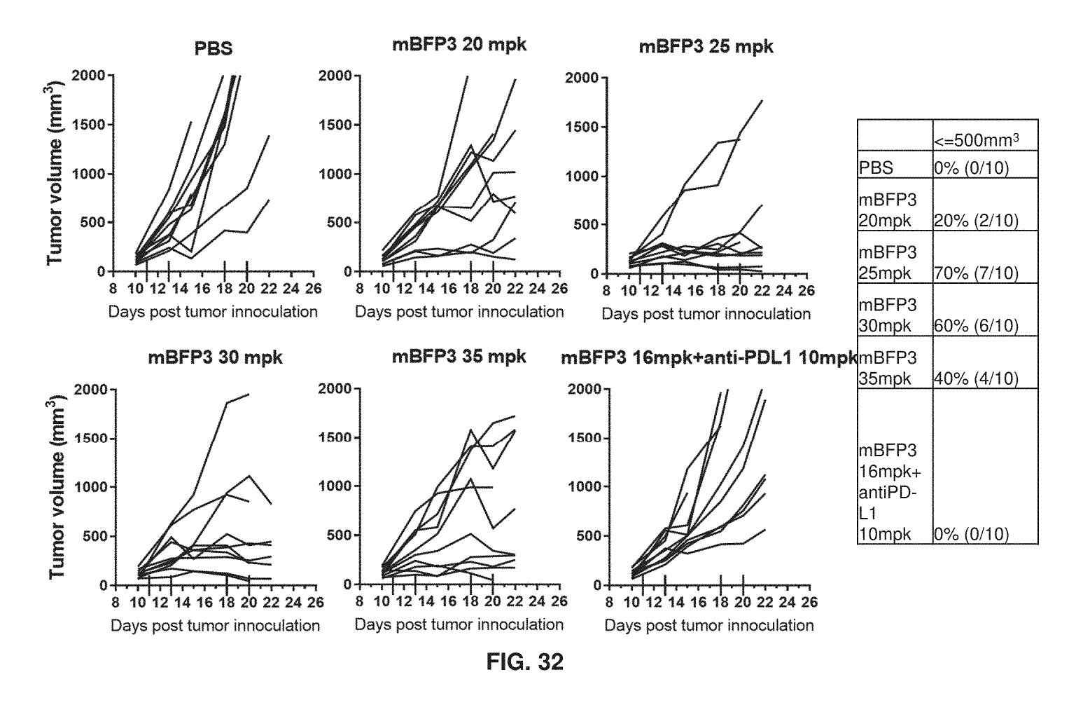

mMEDI7526 was next tested in B16F10 syngeneic mouse model. The model was setup as described in FIG. 31. mMEDI7526, at a dose range from 20 to 35 mg/kg decreased tumor volume and/or delayed tumor growth in the B16-F10 mouse model, compared to PBS controls (FIG. 32). mMEDI7526 at a 25 mg/kg dose had the strongest tumor growth inhibition: at the end of the study 70% mice received 25 mg/kg treatment had a tumor size less than 500 mm³. Thus, mMEDI7526 displayed significant anti-tumor activity in a low responsive tumor model.

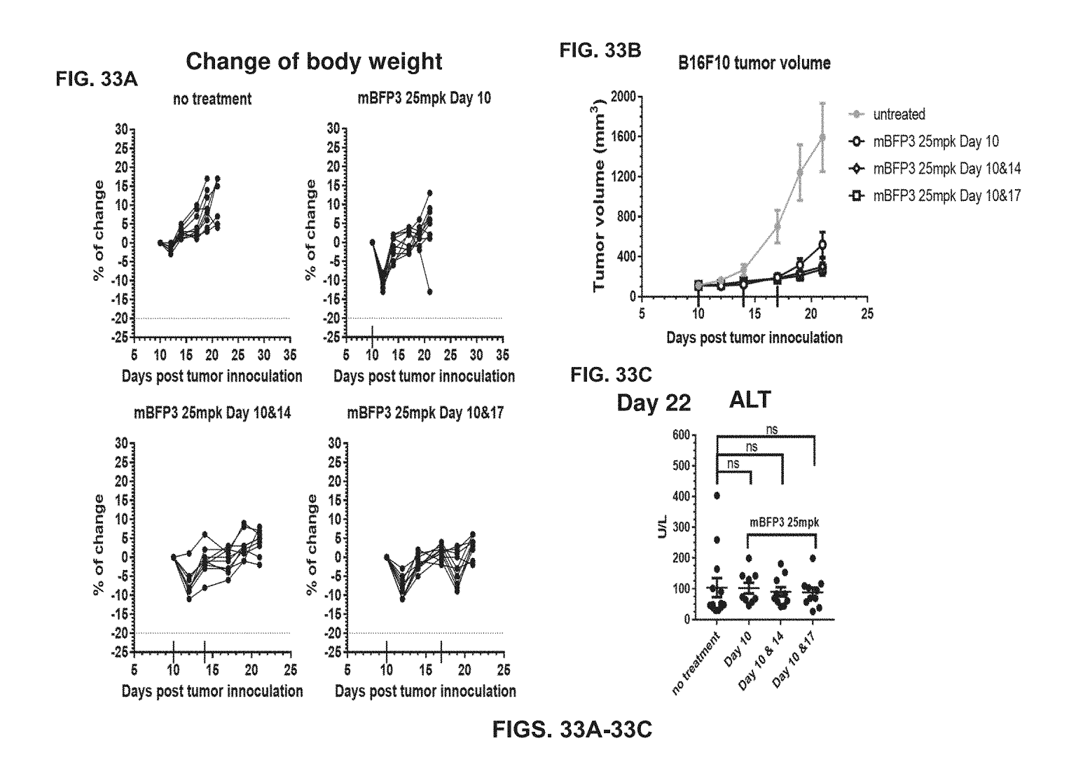

Dosing Optimization. The dosing regimen of mMEDI7526 was optimized as shown in FIG. 33 in the B16F10 model. mMEDI7526 was dosed at 25 mg/kg, either once on day 10 post implantation of B16F10 tumor cells, or twice on day 10 and 14,0020or on day 10 and 17. Treatment of a single dose of mMEDI7526 CD40L-FP significantly decreased tumor volume and/or delayed tumor growth. However, two doses had better anti-tumor activity than a single dose (either day 10 and 14 or day 10 and 17). Additionally, mice treated with mMEDI7526 had no significant weight loss or other observable effects. Thus, reduced dosing frequency of mMEDI7526 can maintain significant anti-tumor activity and reduce major toxicities.

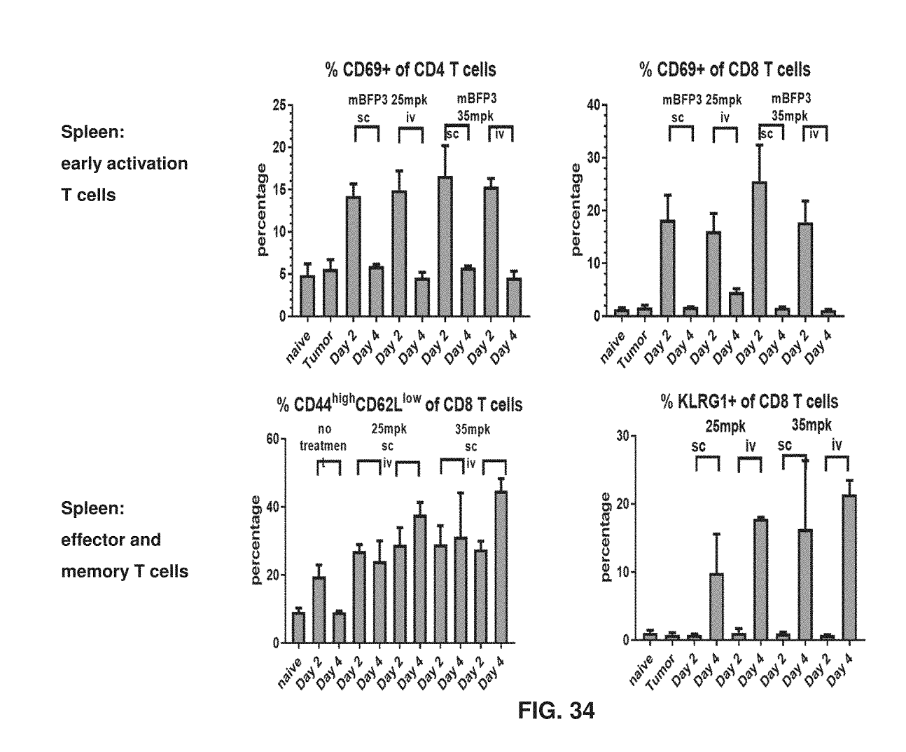

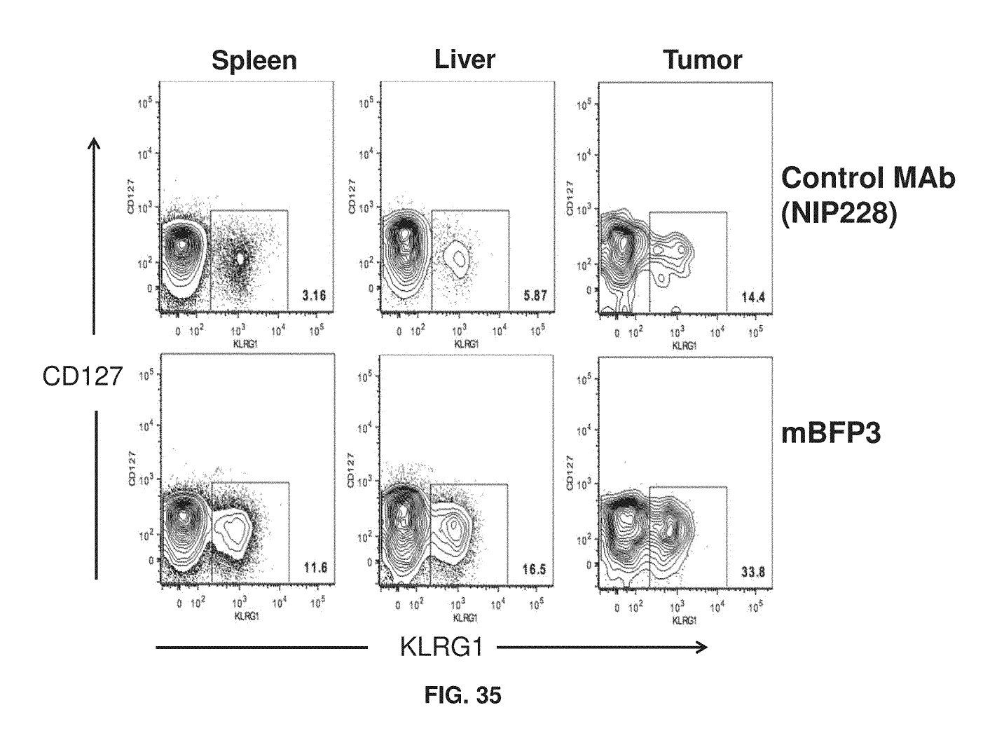

T-cell Activation in vivo. The effect of mMEDI7526 on T cell activation was evaluated in the B16F10 mouse model. mMEDI7526 was dosed at 25 mg/kg on day 10 post-implantation of B16F10 tumor cells and spleen T cells were recovered on days 2 and 4 post-mMEDI7526 treatment. As shown in FIG. 34, treatment of mMEDI7526 led to upregulation of early activation marker CD69 on both CD4+ and CD8+ T cells. Furthermore, on day 4, percentages of effector memory CD8+ T cells (CD44^{high}CD62L^{low}) and effector CD8+ T cells (KLRG1+) were significantly increased. In addition, the percentage of effector CD8+ T cells (KLRG1+) was increased not only in the spleen, but also in liver and tumor (FIG. 35). Together, these data indicate that mMEDI7526 induced robust activation of CD4+ and CD8+ cells in tumor bearing mice.

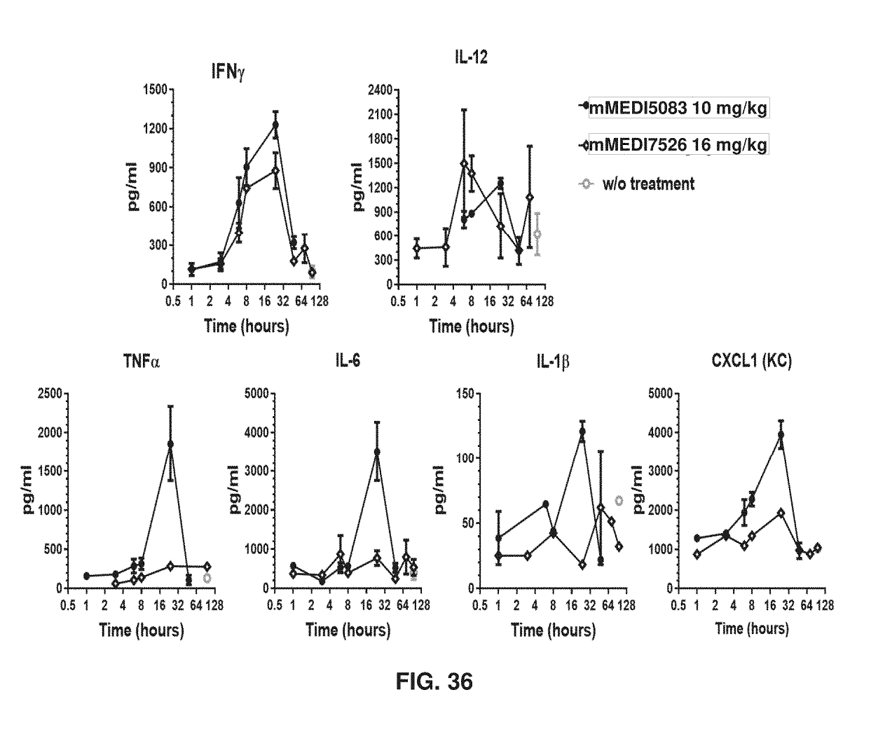

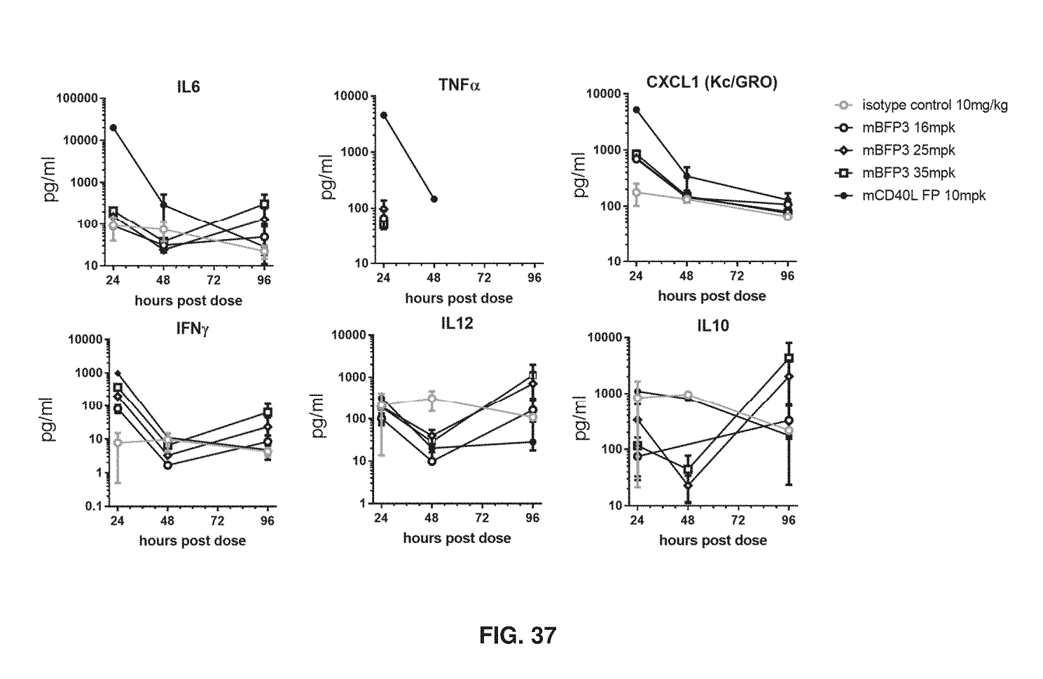

Serum Cytokine Profile Analysis. Changes in serum cytokine profile were monitored post mMEDI7526 and mCD40L treatment. Naive mice were dosed with either mCD40L(mMEDI5083, which is a murinized MEDI5083) at 10 mg/kg or mMEDI7526 at 16 mg/kg and blood was collect at multiple time points post-treatment as indicated in FIG. 36. Serum was separated from whole blood and was subjected to MSD multiple plex analysis (U-PLEX TH1/TH2 Combo) for detecting cytokines, including IFN-γ, IL-1β, IL-2, IL-4, IL-5, IL-6, IL-10, IL-12p70, IL-13, KC/GRO, and TNF-α. Comparing to mCD40L, mMEDI7526 induced similar levels of IFN-γ and IL-12 but significantly less TNF-α and IL-6 (FIG. 37). In a separate study on B16F10 mice, mMEDI7526 at doses between 16 to 35 mg/kg induced much less IL-6 and TNF-α but comparable levels of IFN-γ and IL-12. Thus, mMEDI7526 treatment induces anti-tumor cytokines but limits the production of cytokines that contribute to systemic toxicity.

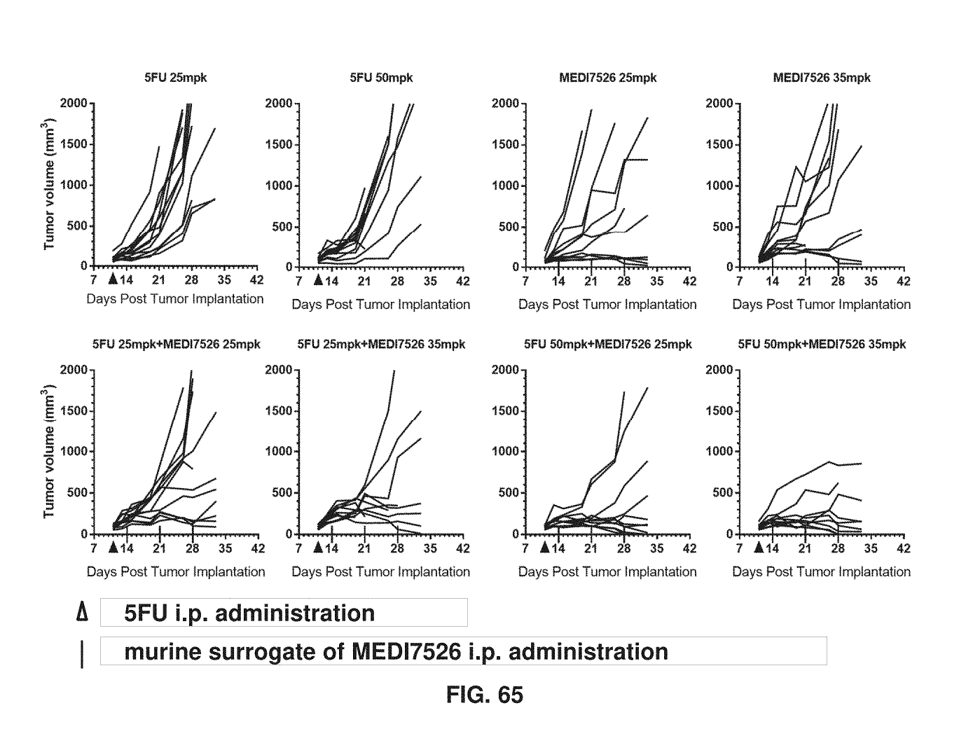

MEDI7526 treatment effectively combines with chemotherapy. In a separate study, the effect of MEDI7526 alone or in combination with chemotherapy Fluorouracil (5FU) for the treatment of CT26 murine tumors was evaluated. A total of 0.5 million CT26 tumor cells were implanted s.c. in mice and tumors were monitored for ˜10 days until they were measured ˜100mm³, at which time mice were then randomized into treatment groups based upon their tumor's volume. The next day, treatment was initiated with 5FU at the dose of 25 or 50 mg/kg or PBS control i.p., followed by either 25 or 35 mg/kg mMEDI7526 i.p. thereafter once a week for 3 weeks. Tumor volumes and body weights were measured twice per week up to the end of the study and the results are shown in FIG. 65. Treatment with 5FU at 25 and 50 mg/kg did not sufficiently inhibit CT26 tumor growth and MEDI7526 alone could inhibit tumor growth only in a subset of mice but not all mice. Combination of 5FU and MEDI7526 led to tumor growth inhibition in more mice than the single treatment. 5FU 50 mg/kg plus MEDI7526 35 mg/kg treatment had the best effect and resulted in an inhibition of tumor growth in all mice in the group. This result suggests that combination with chemotherapy can further enhance anti-tumor activities mediated by MEDI7526.

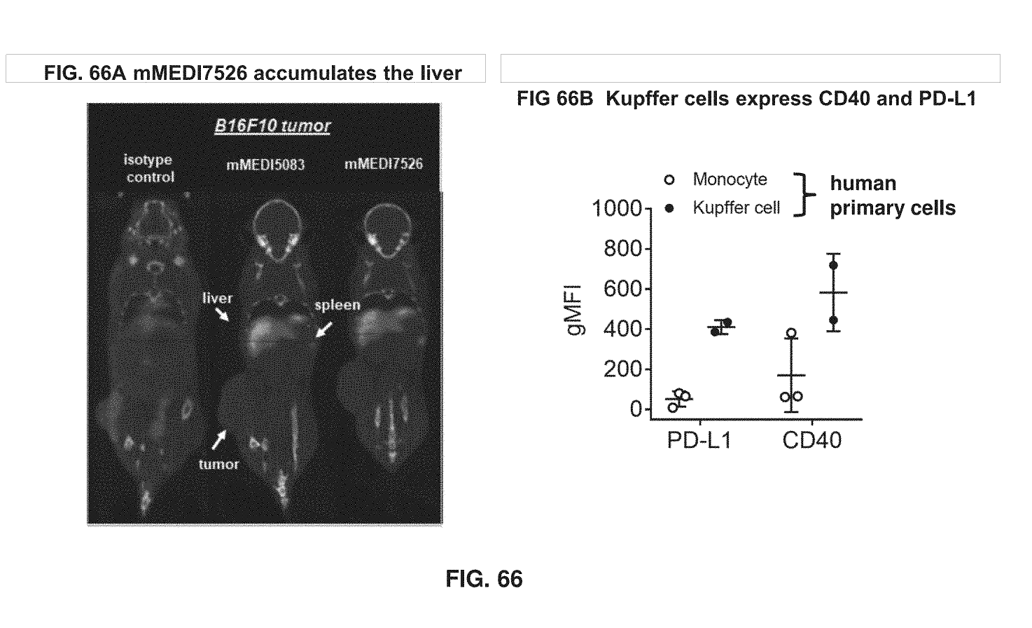

MEDI7526 has the potential to treat liver tumors. The target organs of MEDI7526 were further evaluated. In the biodistribution study, B16F10 tumor cells were implanted in mice, followed by injection of Zr-89 labeled antibodies (isotype control and murine surrogates of MEDI5083 and 7526) as indicated in FIG. 66A. Distribution of labeled antibodies were detected by PETCT. Comparing to the isotype control antibody, both MEDI5083 and MEDI7526 accumulated in the liver and spleen. It was further confirmed that Kupffer cells, a type of liver macrophage express CD40 and PD-L1 (FIG. 66B). These results indicate Kupffer cells in the liver are the target cells of MEDI5083 and MEDI7526.

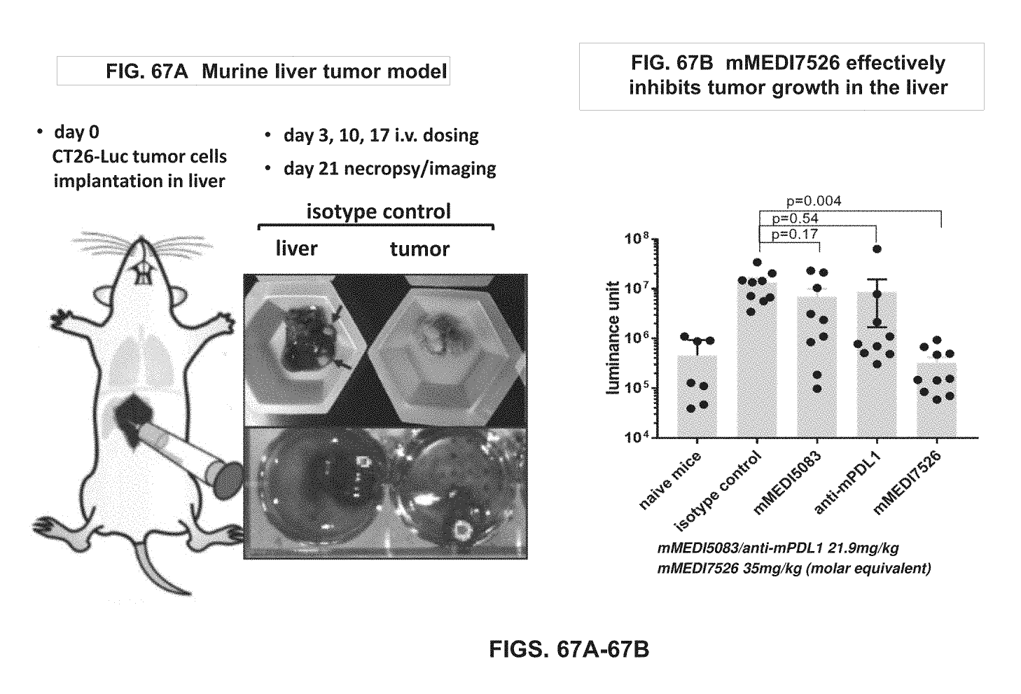

The above results led to further investigation as to whether or not MEDI7526 and MEDI5083 could treat liver tumor in mice. In this experiment, CT26 tumor cells were transfected with luciferase gene and half of million transfected CT26-Luc cells were implanted directly into mouse liver. Mice received CT26 cells and were allowed to recover from surgery and were dosed with isotype control antibody, MEDI5083, anti-PDL1, MEDI5083 plus anti-PDL1 or MEDI7526 on day 3, 10 and 17 post tumor implantations. All test reagents were dosed at 21.9 mg/kg, except MEDI7625 was dosed with molar equivalent 35 mg/kg. On day 21, mice were sacrificed, and liver were recovered for the measurement of luciferase activity, which is an indicator of tumor burden in the liver. Only MEDI7526 treatment effectively reduced tumor burden to the level of tumor-free mice (FIG. 67), indicating MEDI7526 has the potential to treat liver cancer.

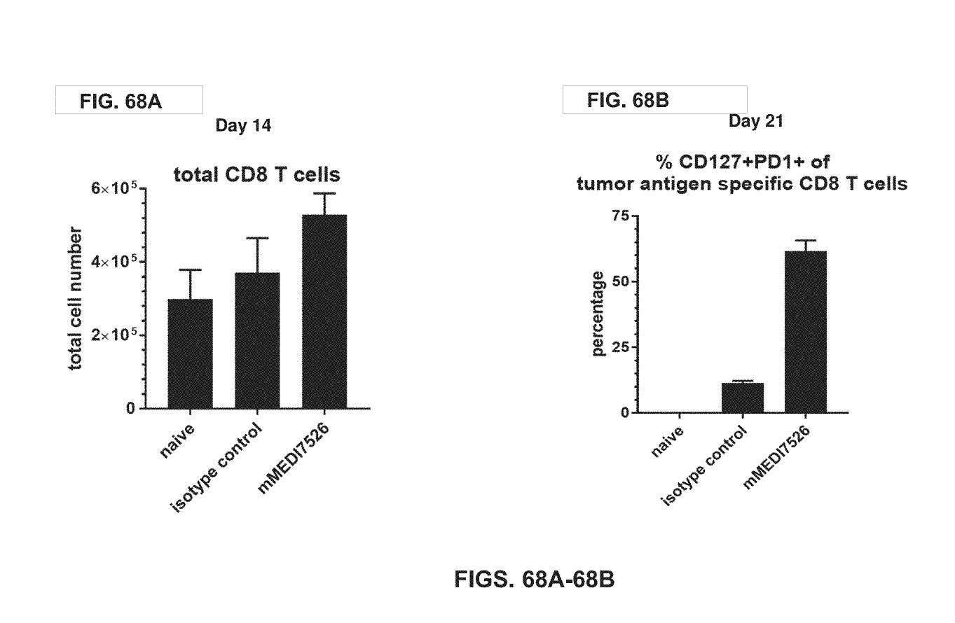

Furthermore, MEDI7526 treatment were associated with increased number of CD8 T cell on day 14 and more activated phenotype among antigen specific CD8 T cells on day 21, as determined by flow cytometry analysis (FIG. 68).

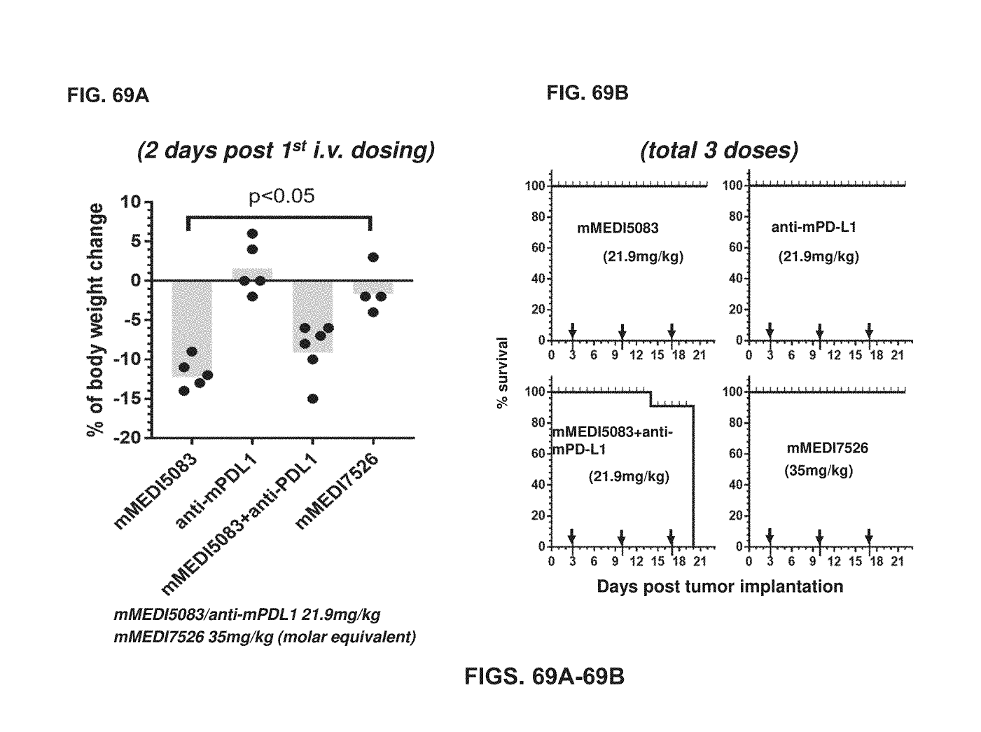

The safety profiles of test reagents were evaluated in the CT26 liver tumor model (FIG. 69). Significant loss of body weight, an indication of severe stress and death of mice were documented. On day 2 post 1^{st} dosing, all mice in the mMEDI5083 group and mMEDI5083 plus anti-PDL1 groups had around 10% loss of body weight. In comparison, none of mMEDI7526 dosed mice showed >5% loss of body weight at the same time point (FIG. 69A). Moreover, all mice received the treatment of mMEDI5083 plus anti-PDL1 were found dead after 3 doses (FIG. 69B). Together, these data further indicate that mMEDI7526 is more tolerable than mMEDI5083 alone or in combination with anti-PDL 1.

### Example No. 18. OX40L Activation of NF-KB

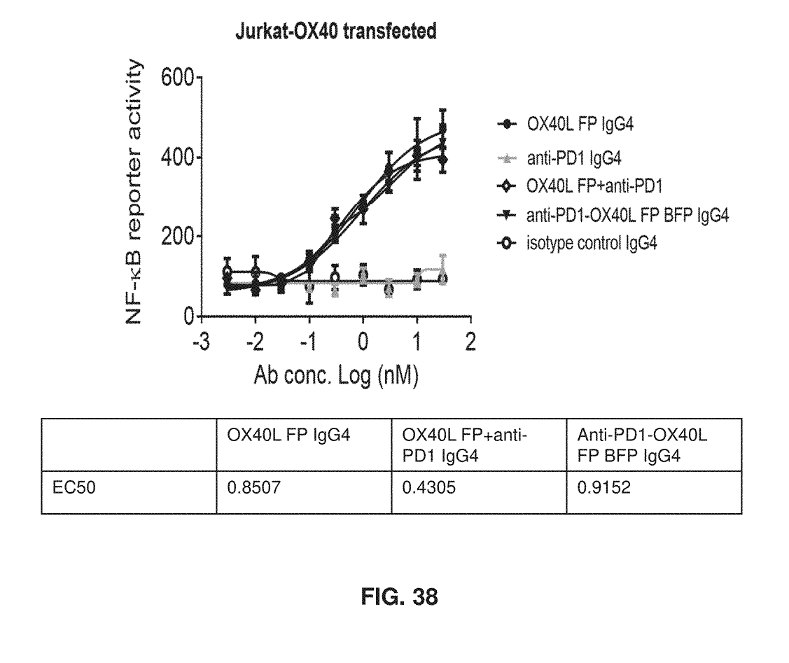

Jurkat NF-κB-Luc reporter T-lymphocytes transfected with OX40 protein were maintained in RPMI1640 (GIBCO) plus 10% HI-FBS (GIBCO), and 1% Pen Strep (GIBCO). On the day of experiment, cells were harvested, adjusted to 2×10⁶ cells/mL and added in a volume of 90 μL per well to a U-bottom 96-well plate (Corning). Ten (10) μL of test reagents (10×) prepared in complete RPMI1640 were added to each well, and the cells were placed in a 37° C. incubator for 4 hours. Luciferase reagent (Steady-Glo® Luciferase Assay Substrate; Promega) was prepared, allowed to equilibrate to room temperature, and added (100 μL) to each well. The plates were placed at room temperature for 5 min followed by immediate read on a SpectraMax® M5 plate reader. FIG. 38 demonstrates that anti-PD1-OX40L BFP activated the NF-κB pathway in Jurkat cells transfected with OX40.

In a separate study, Jurkat NF-κB-Luc reporter cell line engineered to express human OX40-GITR fusion protein with OX40 ectodomain and GITR intracellular domain (Jurkat/OX40-GITR-FP2) were assayed for NF-κB- activity as shown in the previous paragraph. FIG. 71C demonstrates that PD1/OX40L (all subunits of OX40L have wild type protein sequences) induced robust NF-κB activation, however, both PD1/OX40L 1WT and PD1/OX40L 2WT failed to activate NF-κB in the Jurkat reporter cells. These data confirm that all of six residues of F180 on OX40 are critical for activation of OX40 downstream signaling and indicate that PD1/OX40L 1WT and 2WT have minimal OX40 mediated T cell activation function.

### Example No. 19. Mouse Studies of Anti-PD-L1-OX40L BFP

A murine OX40 ligand IgG1 fusion protein (mOX40L FP) was generated that binds to mouse OX40 and triggers OX40 signaling, and was used as a mouse OX40 agonist surrogate for MEDI6383, a human OX40 ligand IgG4P fusion protein. See U.S. Pat. No. 9,718,870. Clone 80 is a rat chimeric mouse IgG1 D265A antibody against mouse PD-L1. The antitumor activity of mOX40L FP and clone 80 was evaluated as a monotherapy or as a combination therapy in mice bearing tumors that originated from MCA205, a mouse syngeneic sarcoma cell line, and in CT26, a mouse colon adenocarcinoma cell line.

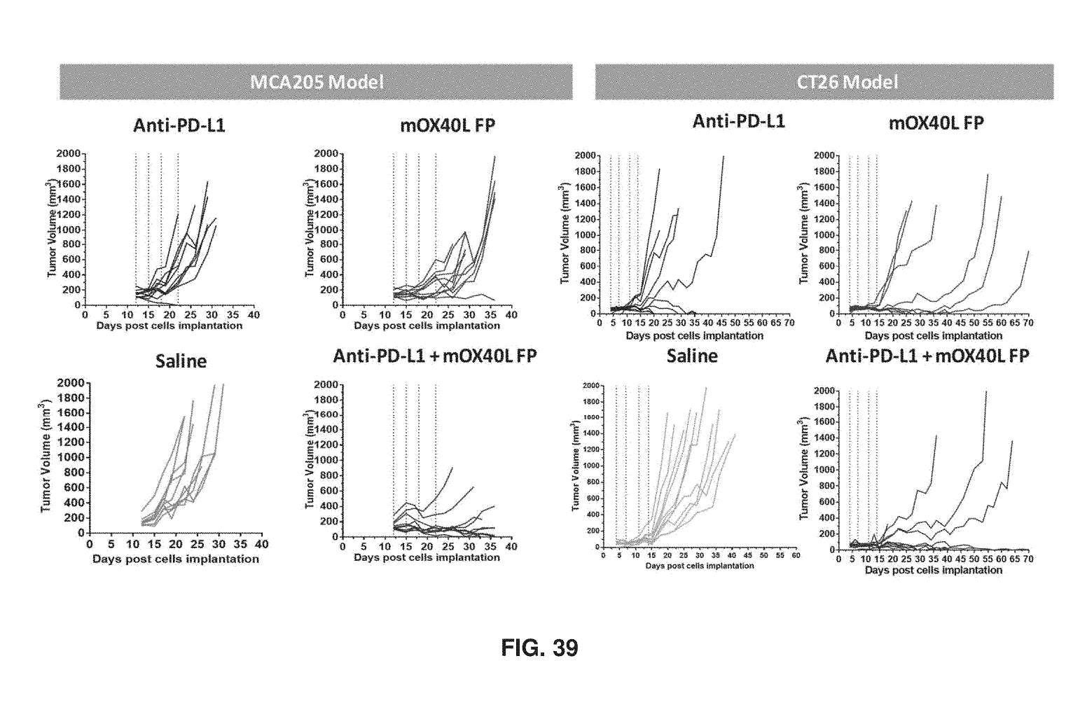

Ten C57BL/6 or Balb/c mice in each group were inoculated SC on Day 1 with MCA205 (left panels) cells or CT26 (right panels) cells, respectively. Control article (saline) and the test articles anti-PD-L1 clone 80 mAb (10 mg/kg), mOX40L FP (9.8 mg/kg), or the combination of 10 mg/kg anti-PD-L1 mAb and 9.8 mg/kg mOX40L FP were administered IP on Days 12, 15, 18, and 22 for MCA205 and on Days 4, 7, 11, and 14 for CT26. Individual tumor volumes over time are shown in the graphs in FIG. 39. IP=intraperitoneal; SC=subcutaneous.

Administration of mOX40L FP in combination with clone 80 resulted in greater antitumor activity than administration of control articles or the agents alone (FIG. 39). Thus, OX40 agonist and PDL1 antagonist therapy provide complementary antitumor benefit in preclinical models.

Bispecific molecules consisting of PDL1-binding moieties may increase retention time in PD-L1+ tumors as compared to bispecific molecules that do not bind to PD-L1. To test this hypothesis, an in vivo biodistribution study was conducted in mice.

Bispecific molecules were conjugated with a chelator (ITC-DTPA, Macrocyclis, Dallas, Tex.) to enable ¹¹¹Indium radiolabeling (Brom et al., 2012), aiming at a specific activity of about 600 MBq/mg. After purification through a desalting column (PD-10 EconoPac, BioRad), radiochemical purity (RCP) of the radiolabeled molecules was verified by instant thin layer chromatography to ensure a labelling efficacy with a RCP>95% and stability up to 4 hours at room temperature.

For the biodistribution studies, female nude mice (Envigo) were inoculated subcutaneously with U87-MG cancer cells (1×10⁷ cells in 0.1 mL), and were randomized into the different treatment groups with a mean tumor volume of 0.2 cm³. All randomized mice were intravenously injected with a single dose of radiolabeled molecule (20 μg/0.2 Mbq/kg body weight). Subgroups of animals were then humanely sacrificed at 1 hour, 1 day, and 4 days after radiolabel dosing. To generate biodistribution profiles, organs/tissue (i.e., blood, muscle, lungs, liver, spleen, kidneys, tumor, tail) were collected, weighed, and measured for levels of radioactivity using a gamma counter (Wizard, PerkinElmer) to calculate percentage injected dose (%ID) and %ID per gram of tissue. The biodistribution profiles illustrate the mean percentage of injected dose corrected per gram of tissue (±SEM), and compare the uptake of radiolabeled MEDI5615 and controls in the indicated tissues at 1 hour, 1 day, and 4 days after injection. n=5 per group except n=6 per group for “Day 4 BFP3.” Asterisks illustrate a significant difference (p<0.05) using a two-tailed t-test.

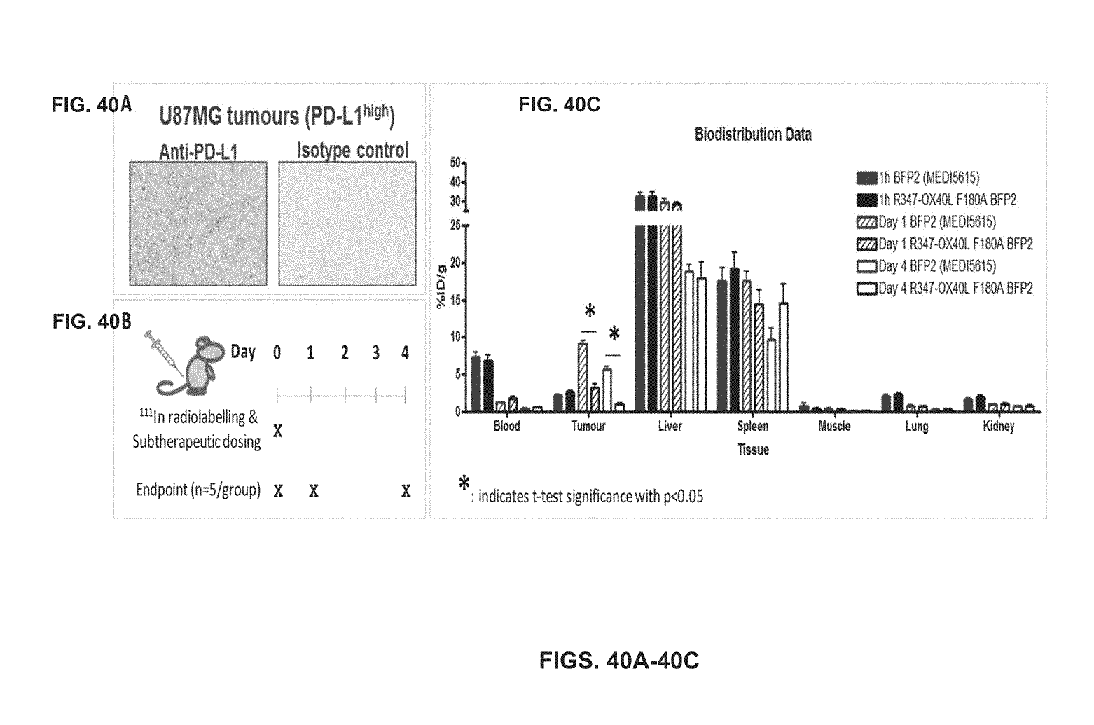

U87MG tumors expressed high levels of PD-L1 as determined by anti-PD-L1 specific immunohistochemistry (FIG. 40A), and were used in the biodistribution studies (FIG. 40B). Subtherapeutic amounts of radiolabeled bispecific molecules were injected into mice bearing U87MG tumors; one targeted PD-L1 and OX40 as an IgG4P BFP2 molecule (MEDI5615; 1×Fc domain), one targeted PD-L1 and OX40 as an IgG1 BFP3 molecule (1×Fc domain), and one was a control article that did not bind to either PD-L1 or OX40 (R347-OX40L F180 BFP2). The bispecific molecules were first detected in the blood after 1 hour and were rapidly cleared so that 1 day later little to no radiolabel could be detected in the blood. MEDI5615 and the control article also distributed rapidly to the liver and spleen independent of target-binding, and remained in these tissues through day 4 (FIG. 40C). In contrast, MEDI5615 penetrated and was retained in the tumor as compared to the control article whilst both molecules were cleared from the blood.

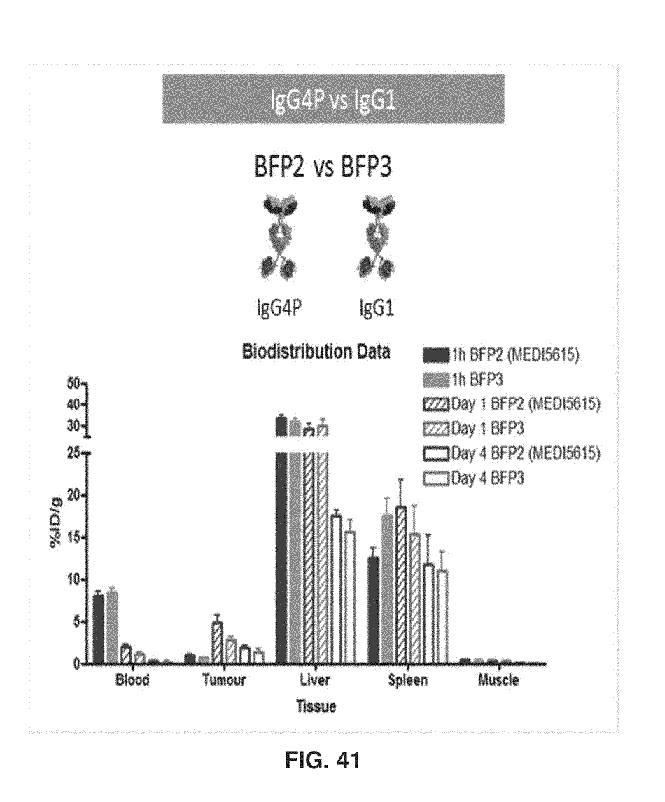

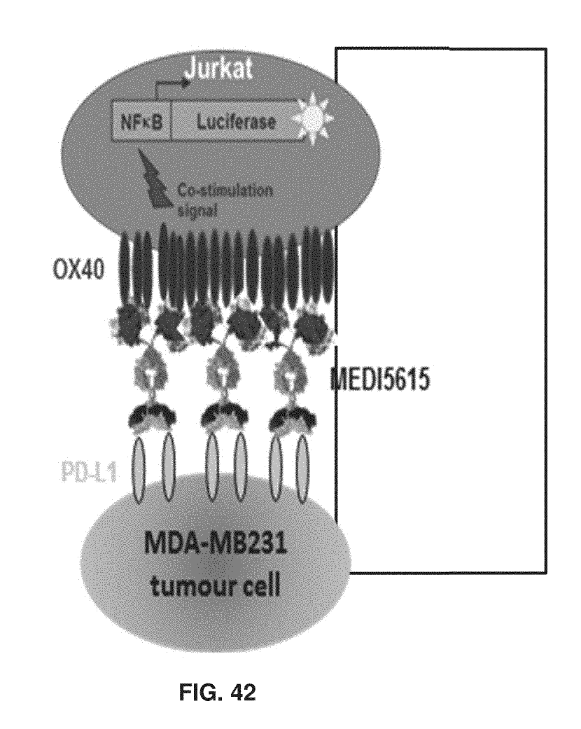

PD-L1/OX40L BFP3 molecules demonstrated an ability to remain in the tumor similarly to MEDI5615 (FIG. 41) suggesting that the tumor retention was independent of the Fc domain and molecule format. The observed differences in tumor update at days 1 and 4 between MEDI5615 and the PDL1/OX40L BFP3 molecules as compared to the control article suggests that tumor retention is mediated by the molecules binding to PD-L1.

### Example No. 20. Activity of Anti-PD-L1-OX40L BFP

The ability of MEDI5615 (PD-L1/OX40L BFP2 bispecific molecule) to activate signaling through human OX40 was assessed in a set of two-cell reporter bioactivity assays, using Jurkat NF-κB-luciferase T-cell reporter lines genetically engineered to express human OX40 (FIG. 42). PD-L1-mediated drug cross-linking occurred through MDA-MB231 cells that expressed cell surface PD-L1. Fcγ receptor-mediated drug cross-linking occurred through HEK293 cells engineered to express Fey receptor IIa (CD32A). T-cell activation was measured as increased luciferase activity in response to stimulation of the NF-κB signaling pathway downstream of primary human T-cell activation. NF-κB signaling occurs downstream of OX40 signaling, and has been reported to correlate with other measures of T-cell activation such as proliferation and cytokine release. Bioactivity was measured for soluble MEDI5615, as well as MEDI5615 incubated with MDA-MB231 cells that expressed cell surface PD-L1 or HEK293 cells engineered to express an individual Fcγ receptor.

Prior to use, OX40 Jurkat reporter cells were cultured in complete RPMI medium in a tissue culture incubator at a density of 0.5-1.5×10⁶ per mL. Cells were passaged the day prior to the bioassay at a density of 10⁶ cells per mL. OX40 Jurkat reporter cells, MDA-MB231 cells, and CD32A HEK cells were collected and pelleted. Bispecific molecules were serially diluted 3-fold in complete RPMI. OX40 reporter cells plus presenting cells were added to a 96 well plate at 100,000 cells per well. The bispecific molecule was added to cells in complete RPMI media, to a final concentration starting at 1 μg/mL and diluted, as described above. After 16-24 hours incubation time 100 μL reconstituted Steady-Glo® luciferase assay solution (Promega, Madison, Wis.) was added to each well and mixed to lyse cells and then incubated to equilibrate luciferase signal. Steady-Glo®/sample lysate (150 μL) was transferred from each well to a 96 well, white walled assay plate for detection and luminescence read using a Perkin Elmer Envision™ luminescence reader. GraphPad Prism for Windows (GraphPad Software, San Diego, Calif.), was used to plot the concentration of the bispecific molecule (x-axis is log10 of protein concentration) versus luminescence RLU (y-axis).

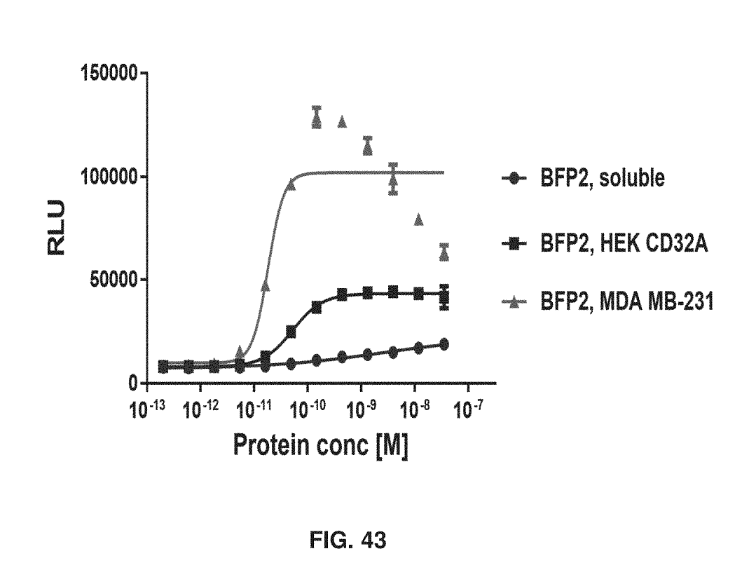

As seen in FIG. 43, MEDI5615 activated the OX40 signaling pathway, as measured by NF-κB signaling, in human OX40-expressing Jurkat T cells in the presence of cells that express Fcγ receptor (CD32A-expressing HEK293 cells) and PD-L1 (MDA-MB231 cells), with EC50 values of 52 pM and 18 pM, respectively. In the absence of cells that express PD-L1 or Fcγ receptors capable of cross-linking MEDI5615, minimal reporter cell-line activity was measured.

Next, the ability of MEDI5615 to overcome the suppressive function of natural CD4+ CD25+ Treg (nTreg) cells on effector CD4+CD25− T cell proliferation and to reduce the release of IL-10 from nTregs was tested in a human T cell co-culture assay.

Human CD4+ effector and Treg cells were isolated from PBMCs using a human Treg cell isolation kit as per manufacturer's instructions (Life Technologies, Paisley, UK). This process involved negative selection of total CD4+ cells by antibody labelling of non-CD4+ cells and then removal of antibody positive cells through use of magnetic bead-based depletion. Treg cells were separated from effector CD4+ cells by labelling with anti-CD25 followed by positive selection with magnetic beads, which were subsequently removed from the isolated cells.

Effector CD4+CD25− T cells were labelled with CFSE using the CellTrace™ CFSE cell proliferation kit (Life Technologies, Paisley, UK). Effector T cells and Treg cells were co-cultured for 4 days at 37° C. at a 1:1 or 1:2 ratio in wells of 96-well plates coated with anti-mouse CD3 mAbs and in the presence of soluble anti-mouse CD28 antibody mixed with control and test articles. Cells were restimulated with PMA plus ionomycin in the presence of brefeldin A for an additional 4 hours, fixed, and tested for IL-10 production by flow cytometry using intracellular cytokine staining methods. The percentage of divided effector CD4+ T cells and the percentage Treg cells producing IL-10 at the end of the assay was assessed by flow cytometry.

The percentage of divided effector T cells (CFSE low) was determined; non-viable (eFluor positive) cells and regulatory T cells (CFSE negative) were discriminated, and excluded from the analysis. The percentage of Treg cells producing IL10 was assessed following exclusion of non-viable and effector T cells (CD25 negative).

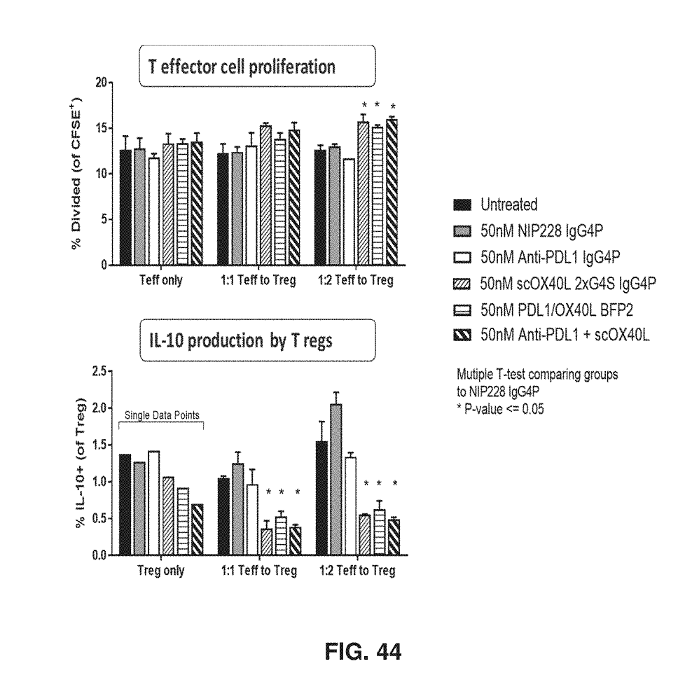

Effector T cells in the absence of Treg cells divided following culture with anti-CD3 and anti-CD28 (FIG. 44, top). The percentage of effector T cells entering the cell cycle did not increase upon the addition of the test articles. Test articles consisting of OX40 agonists (i.e., scOX40L 2×G4S IgG4P, MEDI5615, and anti-PD-L1 IgG4P+scOX40L 2×G4S IgG4P) statistically increased the percentage of divided effector T cells in the presence of Tregs at a 1:2 effector to Treg ratio as compared to control articles (i.e., untreated, NIP228 IgG4P, and anti-PD-L1 IgG4P). No increase in the percentage of effector T cells dividing was observed in cultures with a 1:1 effector to Treg ratio.

Test articles consisting of OX40 agonists significantly reduced the percentage of Tregs producing IL-10 in co-cultures with effector T cells as compared to control articles (FIG. 44, bottom). These results suggest that MEDI5615 functions similarly to OX40 agonists to overcome the suppressive activity and IL-10 production of Treg cells.

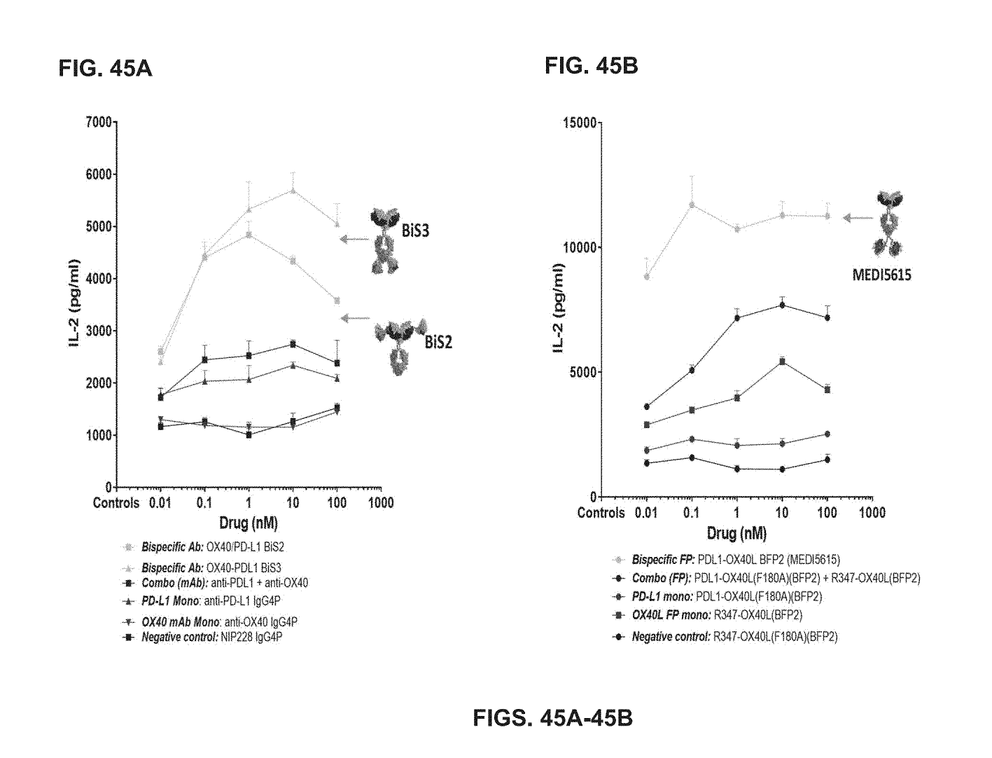

Next, the ability of PD-L1 and OX40-based bispecific molecules to co-stimulate human PBMC in the presence of the superantigen, staphylococcal enterotoxin B (SEB), was assessed. The anti-human CD3 (clone SK7) antibody was pre-coated to wells of a 96-well plate. Human PBMCs were isolated from healthy donors, cultured for 72 hours with anti-CD3, SEB (25 ng/mL), and the indicated test articles and the culture supernatants were tested for IL-2 (FIGS. 45A-B). BiS2 and BiS3 OX40/PD-L1 bispecific antibodies (FIG. 45A; see also U.S. patent application Ser. No. 15/588,271) and MEDI5615 (FIG. 45B) induced human PBMC to produce IL-2 in a concentration dependent manner as determined by an electrochemiluminescent ELISA. The amount of IL-2 produced by the bispecific molecules was greater than the amount of IL-2 produced by human PBMC cultures containing OX40 antibodies (anti-OX40 IgG4P), PD-L1 antibodies (anti-PD-L1 IgG4P), combinations of OX40 and PDL1 antibodies, control bispecific fusion proteins (PDL1-OX40 F180A BFP2, R347-OX40L BFP2) alone or in combination, and negative control articles (N1P228 IgG4P; R347-OX40L F180A BFP2).

Next, cell-based equilibrium binding assays were performed to measure the apparent affinity of MEDI5615 (PD-L1/OX40L BFP2) binding to human and cynomolgus monkey OX40 and PD-L1 expressed on the cell surface of engineered CHO cells.

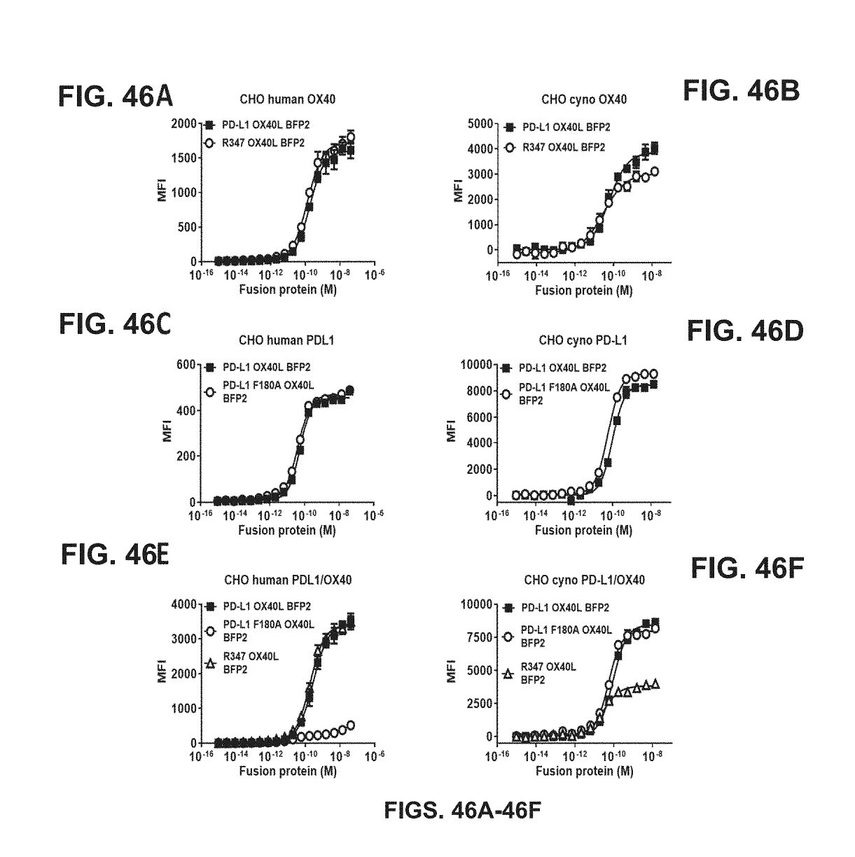

Test articles were diluted over 19 3-fold dilutions in series to CHO cells engineered to express human or cynomolgus monkey OX40, PD-L1 or both OX40 and PD-L1. Cells and test articles (n=3) were incubated for 1 hour at 4° C., washed three times with FACS buffer (PBS+2% heat inactivated newborn calf serum), incubated with AlexaFluor® 647-labeled goat anti-human IgG secondary antibody and propidium iodide (PI), washed with FACS buffer, and analyzed on a flow cytometry. After fluorescence compensation, live (PI negative), single cells were gated and the mean fluorescence intensity (MFI) of secondary antibody was determined to report the level of binding of each test article MFI of test article binding versus fusion protein concentration (M) was plotted to create binding curves from which apparent K_{D} and receptor occupancy values were determined. See FIGS. 46A-F.

The mean equilibrium dissociation constants (K_{D}) for the interaction of MEDI5615 with various CHO cells are reported in Table 4 below.

To determine the apparent K_{D} for test article binding to cells, a non-linear regression (curve fit) equation for one site (specific binding) was employed using the data in FIGS. 46A-F. The results revealed that the mean equilibrium dissociation constants (K_{D}) for the interaction of MEDI5615 with CHO cells that express cell surface human OX40 is 180 pM, that express cell surface human PD-L1 is 88 pM, and that express both human OX40 and human PD-L1 is 270 pM. The K_{D} for the interaction of MEDI5615 with CHO cells that express cell surface cynomolgus monkey OX40 is 56 pM, that express cell surface cynomolgus monkey PD-L1 is 110 pM, and that express both cynomolgus monkey OX40 and cynomolgus monkey PD-L1 is 99 pM. Similar results were obtained using control BFP2 molecules capable of binding only one antigen, OX40 or PDL1.

The 20%, 50%, and 90% human OX40 receptor occupancies at equilibrium for the interaction of MEDI5615 with various CHO cells are reported in Table 5.

ECx values were calculated using GraphPad Prism software from non-linear regression analysis using four-parameter fit sigmoidal dose-response curves of the data presented in FIGS. 46A-F.

For the determination of the concentration at which 20%, 50%, and 90% of receptors were occupied by test article, the concentration values (M) were first transformed using the equation X=log[X], and subsequently the ECAnything (ECf) was determined for f=20, f=50, and f=90 from sigmoidal dose-response (variable slope) binding curves using GraphPad Prism software. ECf is the concentration of test article that gives a response f percent of the way between the bottom and top asymptotes, and represents 20% (f=20), 50% (f=50), and 90% (f=90) receptor occupancy, where the top of the calculated curve represents 100% receptor occupancy.

The concentrations of MEDI5615 required to achieve 20%, 50%, or 90% human OX40 receptor occupancy at equilibrium on the engineered CHO cells were calculated to be 45 pM, 180 pM and 1600 pM, respectively. Additionally, the concentrations of MEDI5615 required to achieve 20%, 50%, or 90% cynomolgus monkey OX40 receptor occupancy at equilibrium on the engineered CHO cells were calculated to be 14 pM, 56 pM and 500 pM, respectively.

The concentrations of MEDI5615 required to achieve 20%, 50%, or 90% human PD-L1 occupancy at equilibrium on the engineered CHO cells were calculated to be 22 pM, 88 pM and 790 pM, respectively. Additionally, the concentrations of MEDI5615 required to achieve 20%, 50%, or 90% cynomolgus monkey PD-L1 occupancy at equilibrium on the engineered CHO cells were calculated to be 27 pM, 110 pM, and 950 pM, respectively.

The concentrations of MEDI5615 required to achieve 20%, 50%, or 90% human OX40 and PD-L1 occupancy at equilibrium on the engineered CHO cells were calculated to be 67 pM, 270 pM and 2,400 pM, respectively. Additionally, the concentrations of MEDI5615 required to achieve 20%, 50%, or 90% cynomolgus monkey OX40 and PD-L1 occupancy at equilibrium on the engineered CHO cells were calculated to be 25 pM, 99 pM and 890 pM, respectively.

Thus, MEDI5615 can bind to cells that express cell surface human and cynomolgus monkey OX40 and PDL1 individually or together.

### Example No. 21. Down-Regulation of PD-1 Protein in Activated Human PBMCS

In this study, the ability of BFPs to down-regulate PD-1 protein in activated human PBMCs was assessed.

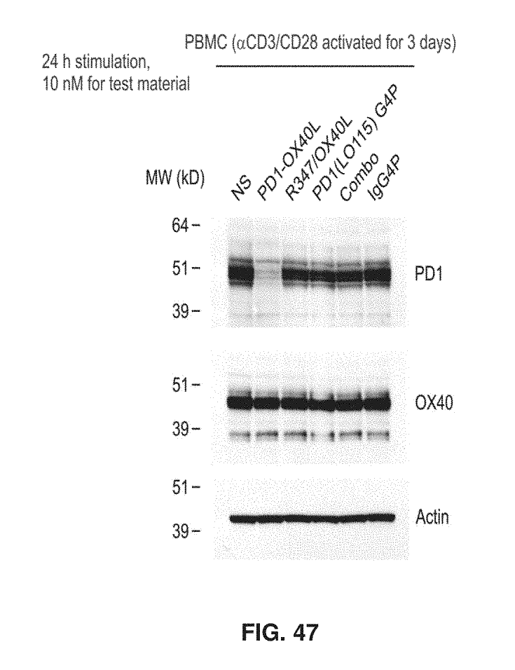

Stimulation and Western protocol: Freshly isolated human PBMCs were resuspended in complete RPMI1640 medium containing 1 μg/mL of anti-CD3 (Clone HIT3a, Biolegend) and anti-CD28 (Clone CD28.2, Biolegend) at 1 million cells/mL (see FIG. 47). After stimulation for 3 days, cells were collected, washed, and resuspended at 1 million/mL in fresh complete RPMI1640 media. Total 2 mL of cells were added to each well of a 6-well plate. Cells were stimulated with 10 nM of test material for 24 h. Cells were lysed in RIPA buffer with protease inhibitors and whole cell lysates were analyzed by immunoblotting. Antibodies used for Western blot are listed in Table 6.

TABLE 6
Western blot analysis to detect the
protein levels of PD1, OX40, and GITR

| antibody | Vendor | Cat# | Dilution |
|---|---|---|---|
| PD-1 (D4W2J) XP ® Rabbit | cell signaling | 86163S | 1:500 |
| mAb #86163 | | | |
| OX40 (D1S6L) Rabbit mAb | cell signaling | 15123S | 1:500 |
| #15123 | | | |
| Human GITR/TNFRSF18 | R&D systems | AB689-100 | 1:500 |
| Antibody | | | |
| anti-actin | Sigma | A2066-.2ML | 1:2000 |
| Peroxidase AffiniPure Donkey | Jackson | 711-035-152 | 1:4000 |
| Anti-Rabbit IgG (H + L) | Immunoresearch | | |
| Peroxidase AffiniPure Donkey | Jackson | 715-035-150 | 1:4000 |
| Anti-Mouse IgG (H + L) | Immunoresearch | | |

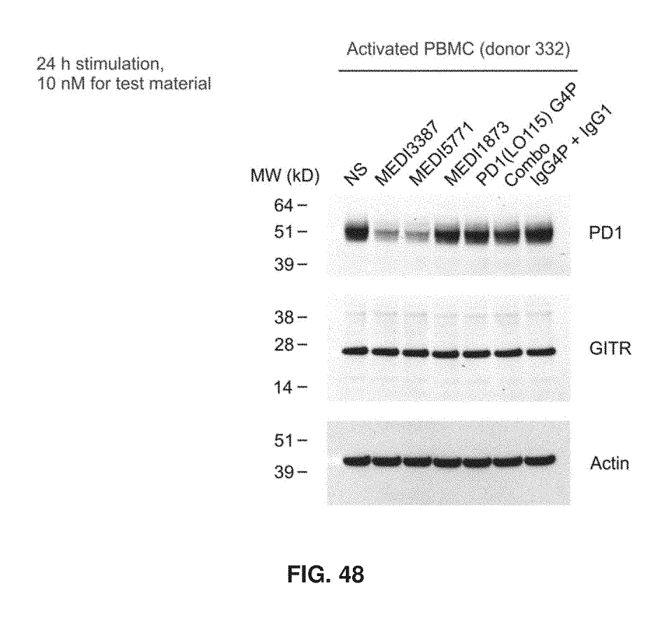

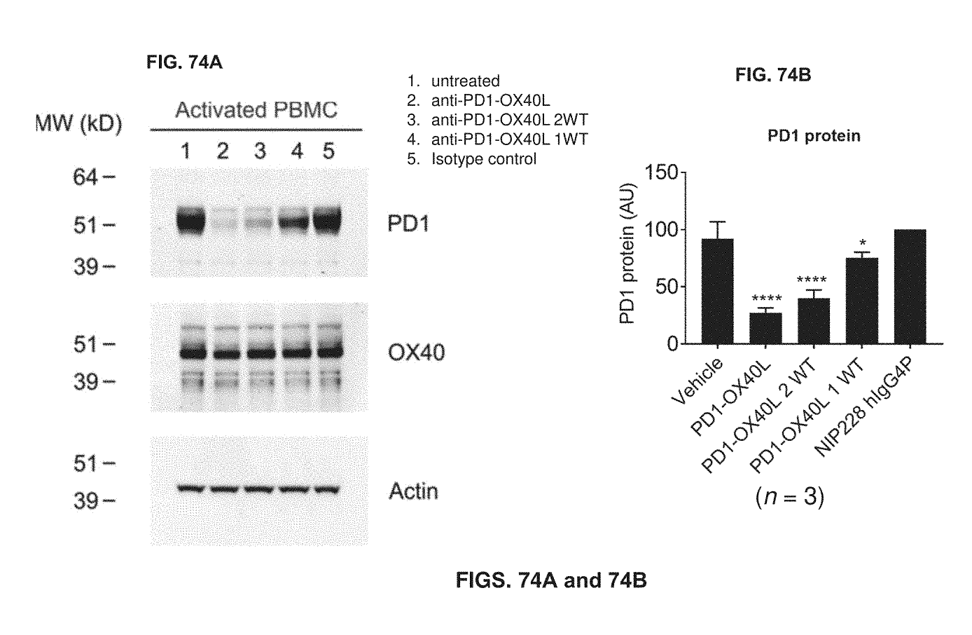

Results are shown in FIGS. 47, 48 and 74. Anti-PD1-OX40L BFP is shown to trigger degradation of PD1 protein in activated human PBMCs (FIG. 47) and anti-PD1-GITRL BFP (MEDI3387) is shown to trigger degradation of PD1 protein in activated human PBMCs (FIG. 48). We found that PD1/OX40L 2WT and 1WT also induced significant PD1 degradation compared to the isotype control antibody (FIG. 74). Therefore, inducing PD1 protein internalization and degradation is driven by OX40 internalization but can be can be independent of OX40 activation.

### Example No. 22. GITRL Activation of NF-KB

This assay utilizes the Jurkat NF-κB Luc FL hGITR clone 29 cell line where engagement of the GITR receptor by GITR ligand induces NFAT promoter driven luciferase activity.

Jurkat-Blue NF-κB/Luc FL hGITR clone 29 reporter T-lymphocytes transfected with GITR protein were maintained in RPMI-1640 plus Glutamax™ (Invitrogen) plus 10% HI-FBS (Invitrogen), 1% Pen/Strep (Invitrogen) media. Cells were harvested, adjusted to 1×10⁶ cells/mL, and added (in 50 μL) to the wells of a flat-bottomed 96-well plate (5×10⁴ cells/well; Falcon). Fifty microliters of 2×test reagents were added to each well. Steady-Glo® (Promega) buffer was defrosted at room temperature prior to use on the day of measurement in the dark. Following 4 hours and 40 minutes incubation the plates were allowed to equilibrate to room temperature for 20 minutes then 100 μL of room temperature reconstituted Steady-Glo® reagent was then added to each well in the 96 well plate and incubated for >10 minutes in a plate shaker prior to measurement. The luminescence readings were measured using the optimized ultrasensitive LUM 96 (opti) 0.1 second read on an Envision plate reader (Perkin Elmer).

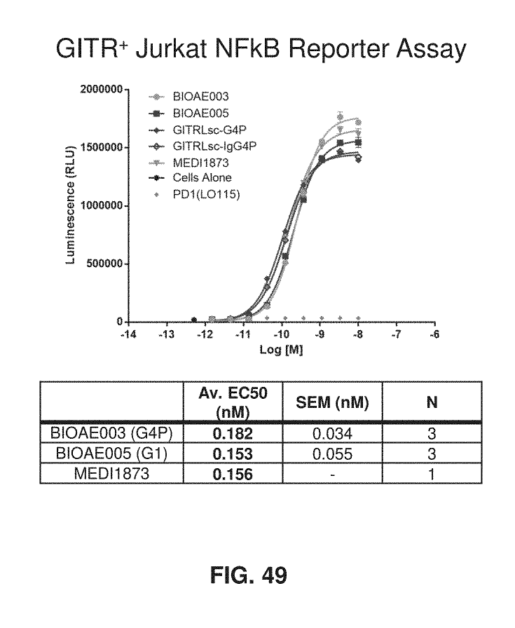

FIG. 49 demonstrates that anti-PD1-GITRL BFPs (MEDI3387 and MEDI5771) activated the NF-κB pathway in Jurkat cells transfected with GITR.

### Example No. 23. Octet Test of PD-1/GITRL Bispecific Concurrent Binding to BIO-HGITR & HPD-1.

The purpose of this experiment was to test the ability of the PD1/GITRL bispecific fusion proteins to simultaneously bind huPD-1 and huGITRL (GITR Ligand) using OCTET Bio-Layer Interferometry to determine interaction.

Materials used this protocol are outlined in Table 7 below.

TABLE 7
Octet assay test materials.

| Reagent | Supplier | Catalogue No. | Batch/Lot No. | Tube size | Storage |
|---|---|---|---|---|---|
| Bio-rhGITR/Fc | R&D | 689-GR-100 | CRS0615071 | 0.1 mg/mL | −80° C. |
| | Systems | | | in PBS + | |
| | Biotinylated in | | | 0.1% BSA | |
| | house 10 May | | | | |
| | 2016 R & D | | | | |
| | Systems | | | | |
| rhPD-1/Fc | R & D | 1086-PD-050 | FVQ0915111 | 50 μg | −20° C. |
| | Systems | | | | |
| rhB7-H1 (PD-L1)/Fc | R & D | 156-B7-100 | FVQ0915111 | 0.5 mg/mL | −20° C. |
| | Systems | | | in PBS | |
| rhGITRL | R & D | 6987-GL-025/CF | TSU0815071 | 0.5 mg/mL | −20° C. |
| | Systems | | | in BSA | |
| Hu IL-7 | Produced & | PS1062 | NA | 144.8 | −80° C. |
| FL_A_H_Biotin | biotinylated in | | | (2.98 mg/mL) | |
| | house (CPL) | | | | |
| NIP228 Human | In house | NA | SP09-410 | 71.0 | −20° C. |
| IgG1 4P | | | | (10.65 mg/mL) | |
| DPBS | GIBCO | 14190-169 | 1734676 | NA | RT |
| 30% BSA | SIGMA | A9576-50ML | SLBJ8273 | NA | RT |
| Tween-20 | SIGMA | P9416-50ML | SLBH9666V | NA | RT |
| High Precision | Fortébio | 18-5117 | 1511211 | NA | RT |
| Streptavidin | | | | | |
| (SAX) dip & | | | | | |
| read biosensors | | | | | |
| Tilted bottom | Fortébio | 18-5076 | 18-0019 | NA | RT |
| (TW384) | | | | | |
| Microplates | | | | | |
| Microplate 96 | Greiner | 655209 | EI5033KF | NA | RT |
| well PP Black | | | | | |

Biotinylated rhGITR/Fc was bound to Streptavidin sensors followed by association of the IO bispecific constructs. Following a brief dissociation phase, the constructs were then tested for their ability to simultaneously bind to rhPD-1/Fc.

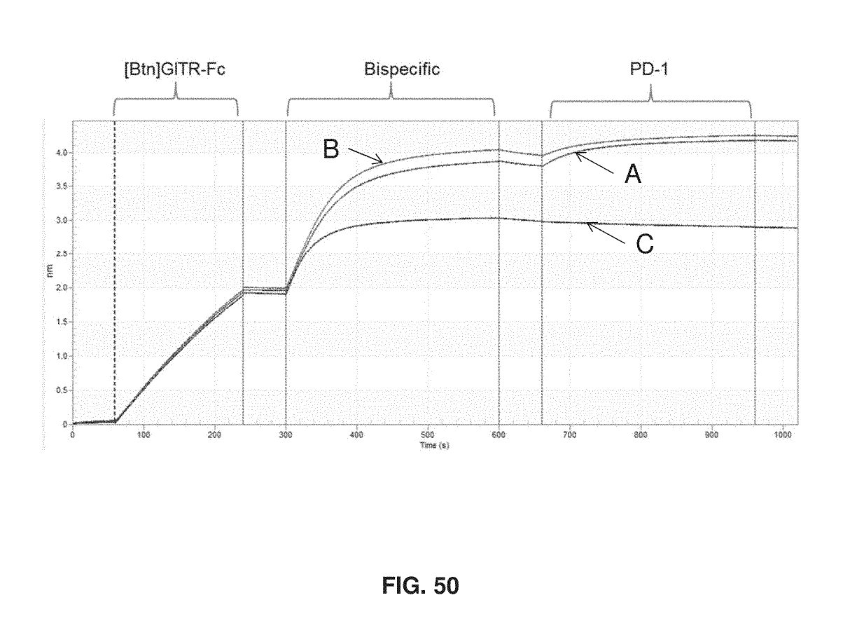

Concurrent binding of bispecific fusion proteins was assessed by Bio-Layer Interferometry (BLI) using the Octet RED384 system and Octet Data Analysis software version 9 (Pall ForteBio). High precision Streptavidin (SAX) dip and read biosensors were first equilibrated in assay buffer (1% BSA, 0.02% Tween 20 in Dulbecco's PBS) for 10 minutes. A baseline was established in assay buffer for one minute prior to capture of biotinylated recombinant human GITR/Fc (689-GR-100, R&D Systems) for 3 minutes. A second baseline was established in assay buffer for 30 seconds prior to capture of the bispecific fusion protein via the GITRL domain for 5 minutes. A dissociation/baseline was performed in assay buffer for one minute before capture of recombinant human PD-1/Fc (1086-PD-050, R&D Systems) for 5 minutes by the antibody component of the bispecific fusion protein. Results are shown in FIG. 50.

### Example No. 24. PD-1 Activity in Activation of NFAT

This assay utilizes CHO K1 OKT3-CD14 (low) hB7H1 (high) cl 2 cells as the antigen presenter cell and Jurkat NFAT Luc2 PD1 clone 3L-B9 cells as the anti-CD3 activated reporter cell line. Inhibition of the PD-1 to PD-L1 interaction by a PD-1 blocking antibody results in activation of the NFAT promoter driven luciferase expression.

Jurkat NFAT Luc2 PD1 clone 3L-B9 reporter T-lymphocytes transfected with PD-1 and CHO K1 OKT3-CD14 (low) hB7H1 (high) cl 2 cells were maintained in RPMI-1640 plus Glutamax (Invitrogen) plus 10% HI-FBS (Invitrogen), non-essential amino acids (Invitrogen) media. Cells were harvested, adjusted to 1×10⁶ cells/mL, and added (in 50 μL) to the wells of a flat-bottomed 96-well plate (5×10⁴ cells/well; Falcon). Fifty microliters of 2×test reagents were added to each well. Steady-Glo® (Promega) buffer was defrosted at room temperature prior to use on the day of measurement in the dark. Following 4 hours and 40 minutes incubation the plates were allowed to equilibrate to room temperature for 20 minutes then 100 μL of room temperature reconstituted Steady-Glo® reagent was then added to each well in the 96 well plate and incubated for >10 minutes in a plate shaker prior to measurement. The luminescence readings were measured using the optimized ultrasensitive LUM 96 (opti) 0.1 second read on an Envision plate reader (Perkin Elmer).

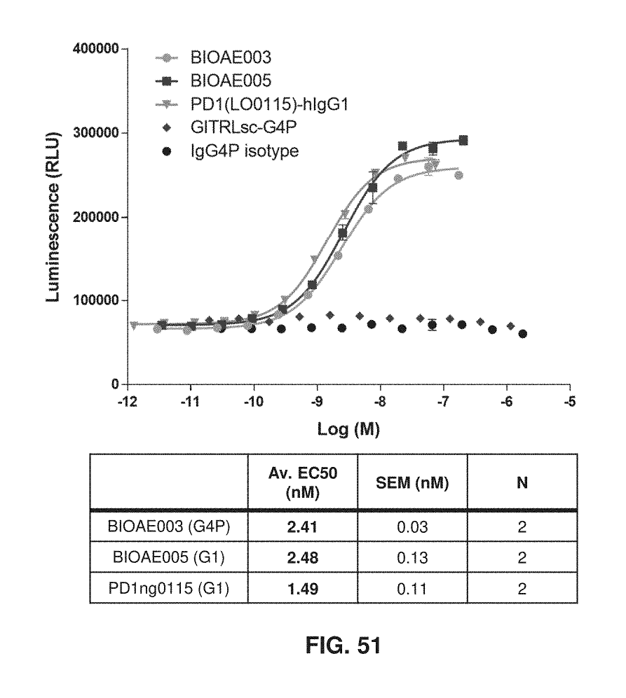

FIG. 51 demonstrates that anti-PD1-GITRL BFPs (MEDI3387 and MEDI5771) activated the NFAT pathway in Jurkat cells transfected with PD-1. These data in concert with those from Example 8 above demonstrate that MEDI3387 and MEDI5771 BFPs demonstrate concurrent binding to human PD-1 and GITR.

### Example No. 25. Anti-PD-1IgG_GITRL in vivo Assays

Mice and tumor models: Eight- to 10-week-old BALB/c or C57BL/6 female mice were obtained from Charles River UK Ltd. or Harlan Laboratories Inc. A 100 mL suspension of CT26 (ATCC) or B16F10 cells in PBS at a cell density of 5×10⁶ cells/mL or 5×10⁴ cells/mL was subcutaneously injected into the right flank of each animal. The B16F10 cell line was implanted in 50% PBS and 50% growth factor-reduced and phenol red-free Matrigel® (Corning). Cell lines were cultured to limited passage before implantation and were periodically screened to confirm the absence of mycoplasma. Cells were further authenticated via STR profiling (IDEXX Bioresearch) and screened for a panel of mouse viruses (Charles River).

Measurable tumors were randomized based on tumor volume into respective groups. The length (mm) and width (mm) of each tumor was measured with an electronic caliper 3 times per week. Volumes of tumors (mm³) were calculated based on the formula [length (mm)×width (mm)²]/2. Tumor growth responses were categorized as a response if there was no measurable tumor or a sustained tumor growth inhibition such that volume was less than 200 mm³ at the end of the study.

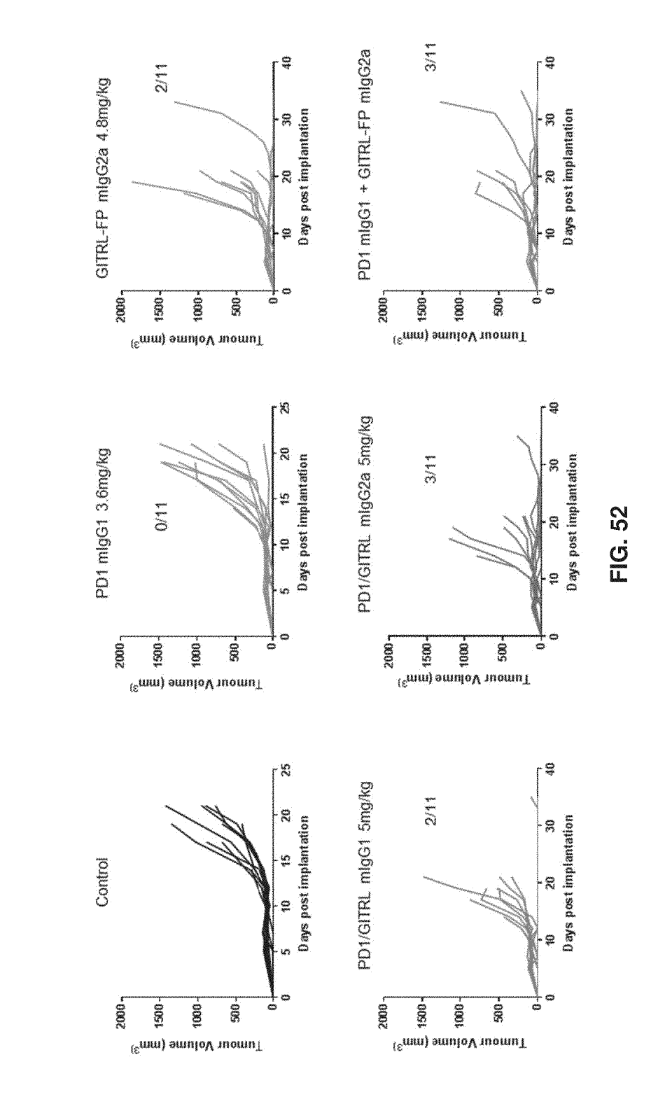

Power calculation was performed to determine group sizes for in vivo studies. Mice were dosed intraperitoneally with either mGITRL-FP, anti-programmed cell death protein-1 rIgG2a (PD-1, clone RMP1- 14, BioXCell), or anti-mouse-PD1/GITRL bi-specific mAb. Mice were dosed with different concentrations depending on the study, starting at day 6 after tumor cell inoculation or when the tumors reached a volume of 200 mm³. Results are shown in FIG. 52.

### Example No. 26. Anti-PD-1IGG_GITRL in vivo Assays

### Pharmacokinetic and Pharmacodynamic (PK/PD) Studies

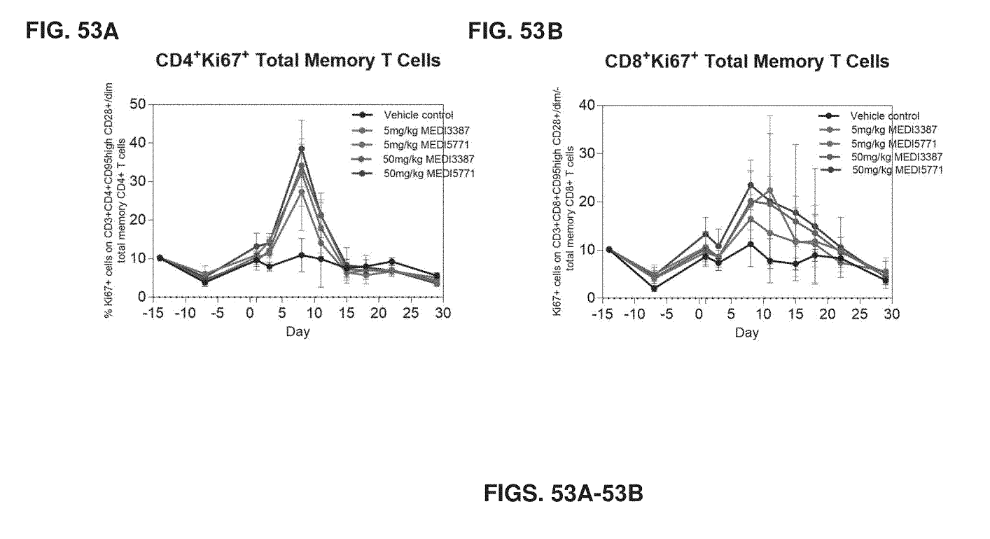

Cynomolgus monkey was considered to be a pharmacologically relevant nonclinical species to test the functional activity of PD-1/GITRL bispecific fusion proteins. The pharmacokinetics (PK) and pharmacodynamics PD-1/GITRL bispecific fusion proteins were assessed in a non-GLP (Good Laboratory Practices) study in cynomolgus monkeys. PK and PD (percent Ki67 positive CD4+ and CD8+ total memory T cells) were evaluated in cynomolgus monkeys (n=5; males) following a single intravenous (IV) dose over the dose range of 5 mg/kg to 50 mg/kg. Blood samples were collected pre-dose and on day 1, 3, 8, 11, 15, 18 22 and 29 post-dose being analyzed on the day of sample collection by flow cytometry. Ki67 results showed a dose-dependent increase in CD4+ and CD8+ total memory T cells (Ki67) (FIGS. 53A-B).

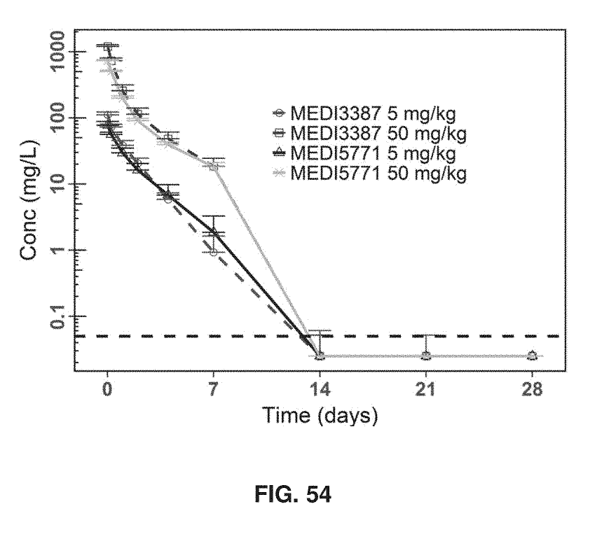

Blood samples were also collected 0.5, 6, 24, 48 and 96 hours post dose on Days 8, 15, 22 and 29 for PK evaluation. In summary, PD-1/GITRL bispecific fusion proteins resulted in approximately proportional increases in Cₘₐₓ and AUC_{0-inf} suggesting linear pharmacokinetics within this dose range (FIG. 54) with a short half-life of 1.12 to 2.19 days (see Table 8).

TABLE 8
Mean pharmacokinetic parameters of PD-1/GITRL bispecific
fusion proteins

| | Dose | | | | AUC_{0-inf} | |
|---|---|---|---|---|---|---|
| | (mg/ | t_{1/2} | Tₘₐₓ | Cₘₐₓ | (day · | Vss |
| | kg) | (day) | (h) | (mg/L) | mg/L) | (mL/kg) |
| MEDI3387 | 5 | 1.12 | 0.50 | 111 | 125 | 61.1 |
| | | (0.278) | (0.00) | (11.7) | (20.1) | (8.33) |
| MEDI3387 | 50 | 1.91 | 0.50 | 1200 | 1050 | 83.2 |
| | | (0.129) | (0.00) | (82.7) | (183) | (8.13) |
| MEDI3387 | 5 | 1.48 | 0.50 | 77.1 | 108 | 88.9 |
| | | (0.481) | (0.00) | (11) | (26.7) | (6.36) |
| MEDI3387 | 50 | 2.19 | 0.50 | 730 | 800 | 135 |
| | | (0.13) | (0.00) | (26.7) | (51.4) | (5.86) |

Values are presented as Mean (Standard Deviation).
AUCₗₐₛₜ = area under the concentration time curve up to the last measurable concentration;
AUC_{INF} = area under the concentration time curve up to infinite time;
Cₘₐₓ = maximum observed concentration;
CL: systemic clearance;
T_{1/2} = half-life;
Vss: terminal phase volume of distribution.

MEDI3387, mean Cₘₐₓ, values were 111 and 1200 mg/L, and mean AUC_{0-inf} values were 125 and 1050 mg·day/L for 5, and 50 mg/kg dose, respectively. Dose normalized AUC values were approximately similar for two dose groups. Mean AUC_{0-inf}/dose was 24.9, and 20.9 for the 5, and 50 mg/kg dose levels, respectively. MEDI5771, mean Cₘₐₓ values were 77.1 and 730 mg/L, and mean AUC_{0-inf} values were 108 and 800 mg·day/L for 5, and 50 mg/kg dose, respectively. Dose normalized AUC values were approximately similar for two dose groups. Mean AUC_{0-inf}/dose was 21.5, and 16.0 for the 5, and 50 mg/kg dose levels, respectively.

### Example No. 27. Anti-PD-1_IGG_GITRL BFPs Enhance T Cell Effector Function in a Primary Human T Cell Reactivation Assay

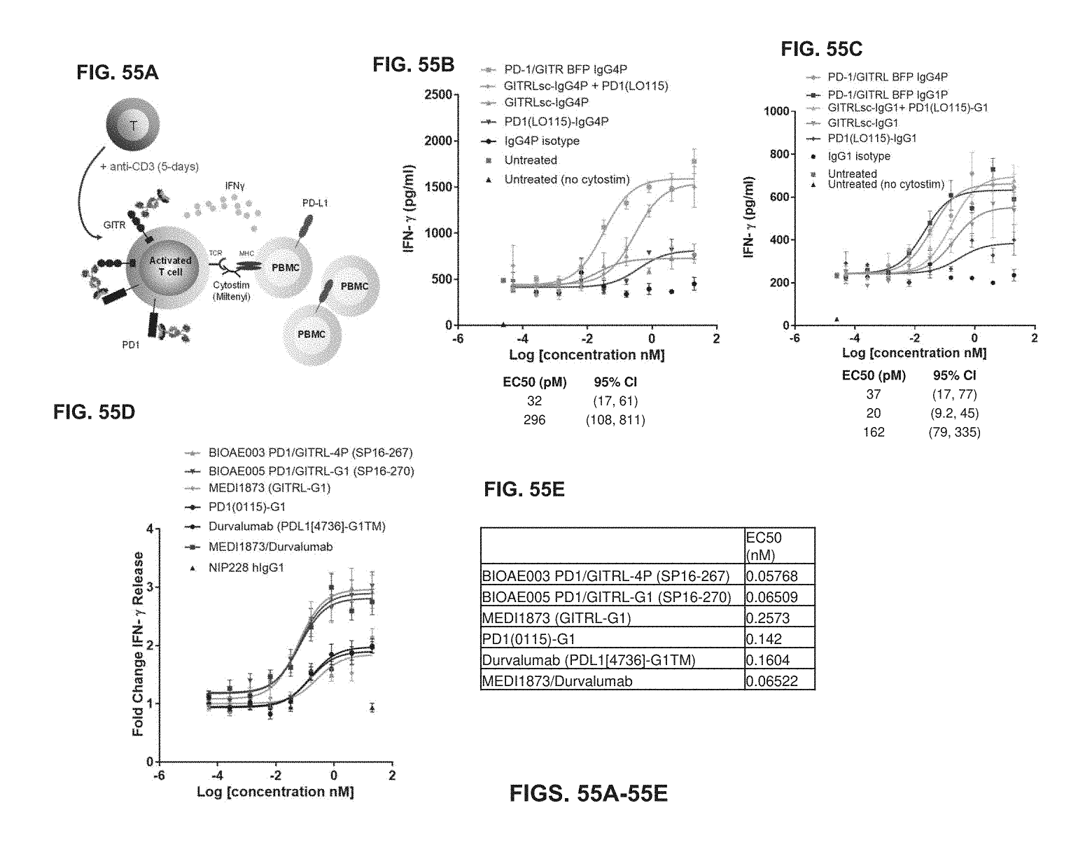

A schematic of the Cytostim T cell Reactivation Assay is shown in FIG. 55A.

PBMCs were prepared from Leukocyte cones (supplied by the NHSBT, Addenbrooke's Hospital) using Ficoll-Paque PLUS (GE Healthcare 17-1440-02), following the manufacturer's recommended protocol. T cells were isolated by negative selection from the donor PBMC using a T cell enrichment kit (Stemcell cat: 19051) following the manufacturer's recommended protocol. The T cells were then resuspended at a concentration of 1E6 cells/mL in T cell media (RPMI1640 with Glutamax™ [Gibco] supplemented with 5% human AB serum [Sigma] and 1% penicillin/streptomycin), and stimulated for 96 hours at 37° C., 5% CO₂, in 6 well tissue culture plates (Costar, cat: 3506) coated previously with an anti-human CD3 antibody (clone OKT3, Ebioscience cat: 16-0037-85). To coat the 6 well plates, 1 mL of PBS containing 0.2 μg of OKT3 was added to each well and incubated overnight at 4° C. Plates were washed twice with PBS prior to use.

T cells were then washed and incubated in fresh T cell media for a further 24 hours at 37° C., 5% CO2 without stimulation. These ‘rested’ T cells were subsequently mixed at a ratio of 1:4 with autologous PBMCs that had been depleted previously of T cells using a using EasySep™ Human CD3 Positive Selection Kit II (Stemcell, 17851) following the manufacturer's recommended protocol. The resulting cell mix was aliquoted onto 96 well U-bottomed tissue culture plates (Costar 8797BC) in T cell media, supplemented with human Cytostim (Miltenyi Biotec, 130-092-173) at a concentration of 1 in 400, together with test drugs or control mAbs. Each reaction had a total volume of 200 μL and contained 5E5 cells. Reactions were incubated for 72 hours at 37° C., 5% CO₂, after which supernatants were removed and tested subsequently for INF-γ release by ELISA.

Results are shown in FIGS. 55B-C. MEDI3387 and MEDI5771 are >4× more potent than a combination of monospecific (PD-1 and GITRL) molecules and therefore illustrate an unexpected synergistic effect due to the BFP format. The in vitro data described above (FIGS. 55D-E) demonstrate bioequivalence for MEDI3387 and MEDI5771 to a combination of either GITRL (MEDI1873)/Durva (MEDI4736) or MEDI1873/aPD-1 mAb (LO115). In vivo data demonstrate bioequivalence to a combination of MEDI1873/aPD-1 mAb.

### Example No. 28. Fluorescence Biodistribution of GITR in vivo

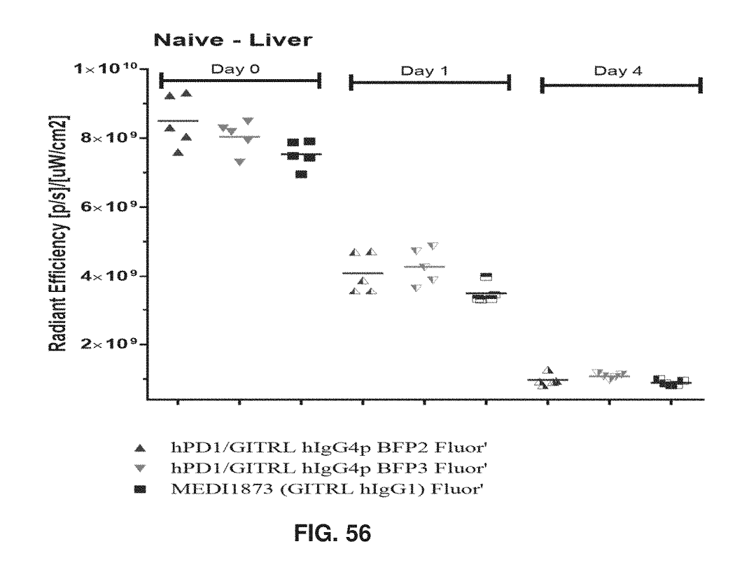

Female SCID mice, aged 6/8wks, were injected subcutaneously with human lung tumor cell line, NCI-H358 (5e5 cells/mouse), on day 0. A single intravenous administration of fluorescent (IRDye 800CW) labelled antibody (10 mg/kg in a dose volume of 100 μL/25 g) occurred at day 25, when tumor size was approximated 200-300 mm³. All three antibodies were labelled with IRDye 800CW, in accordance to the manufactory's protocol two weeks prior to in vivo injection. Five mice per treatment group were anaesthetised and imaged at 1 hour, 24 hours, and 96 hours post dose, using an IVIS spectrum. Optimal wavelength settings were selected for the fluorescent dye, and radiant efficiency values were determined for tumor and liver regions, using the PerkinElmer image analysis software. Results demonstrate that there is no significant difference between teliver biodistribution profiles for MEDI3387, MEDI5771 and MEDI1873 (FIG. 56).

### Example No. 29. Anti-PD-L1-TNF-α BFP Triggers Down-Regulation of Pd-L1 Protein on the Cell Surface of T24 Tumor Cells

T24 is a human urinary bladder transitional carcinoma cell line that constitutively expresses both TNF-α receptors and PD-L1. In this study, T24 cells were mixed with testing materials at a 10 nM concentration and incubated at 37° C. for 24 hrs. After incubation, free antibodies were removed by washing, and the amount of PD-L1 protein on cell surfaces was quantified with Qifikit (Agilent). Briefly, T24 cells post-treatment were reacted with unconjugated anti-PD-L1 (clone 29E.2A3) on ice for 30 minutes, followed by FITC conjugated F(ab)2 goat anti-mouse IgG for another 30 minutes. Mean fluorescence intensity of FITC signals was recorded, and the amount of PD-L1 antigen per cell on T24 cells was calculated following the manufacturer's instructions.

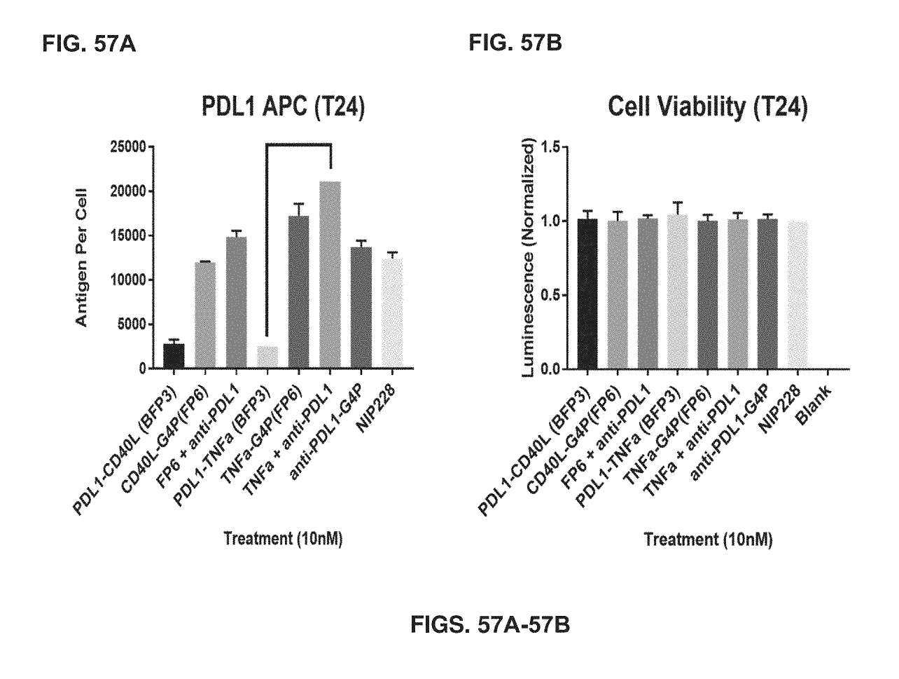

The effect on cell viability was assessed with CellTiter-Glo® cell viability kit (Promega). In this study, 10,000/well T24 cells were cultured in opaque-walled multiwell plates treated as described above and after incubation, T24 cells were mixed with diluted CellTiter-Glo® reagent. The contents were mixed for 2 minutes on an orbital shaker to induce cell lysis, and the plate was incubated at room temperature for 10 minutes to stabilize luminescent signal. Luminescence was recorded on a SpectraMax M5 plate reader. FIG. 57 demonstrates that anti-PD-L1-TNF-α BFP triggers down-regulation of PD-L1 protein in T24 tumor cells. This result demonstrates that another BFP molecule which replaces CD40 with another TNF-α family protein can trigger cell surface PD-L1 downregulation.

### Example No. 29. Anti-PD-L1-TNF-α BFP Triggers Down-Regulation oF PD-L1 Protein on the Cell Surface of T24 Tumor Cells.

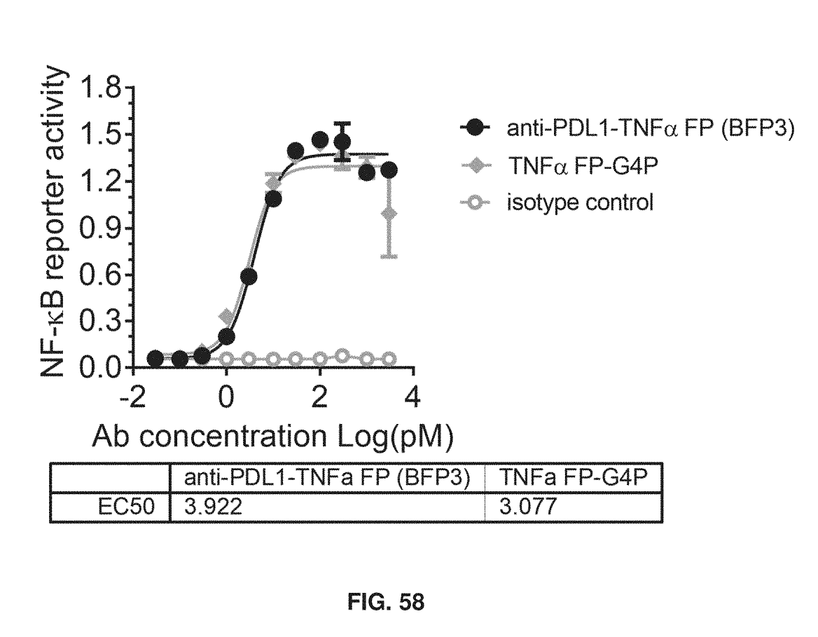

Another assay on THP1-blue cells was setup as described above in Example No. 4, except test reagents were replaced with anti-PDL1-TNF-α BFP3 and TNF-α FP and isotype control. FIG. 58 demonstrates anti-PDL1-TNF-α BFP activate NF-κB pathway on THP1 myeloid cells.

### Example No. 30. Anti-PD-1-OX40 BFP Drives Internalization

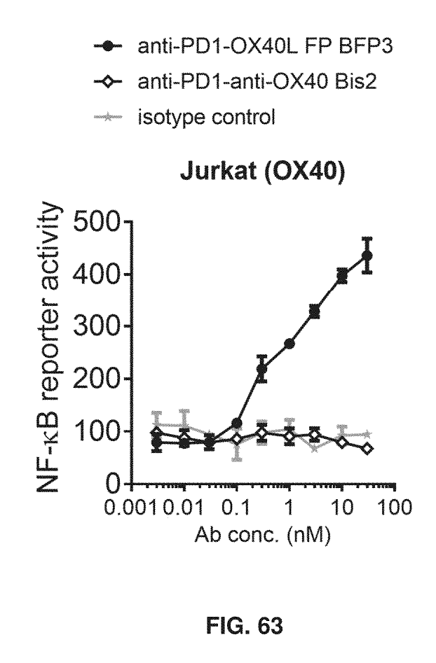

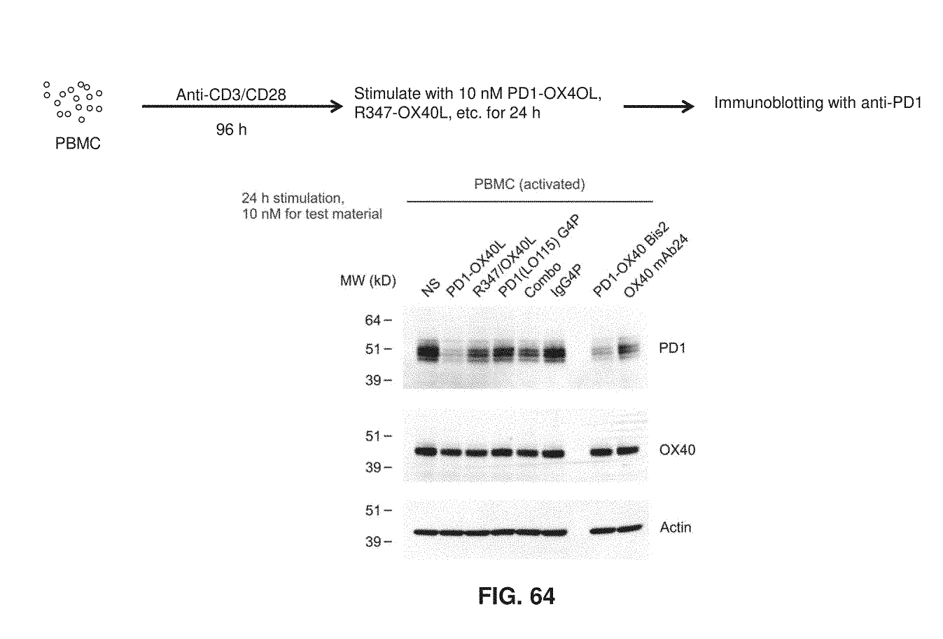

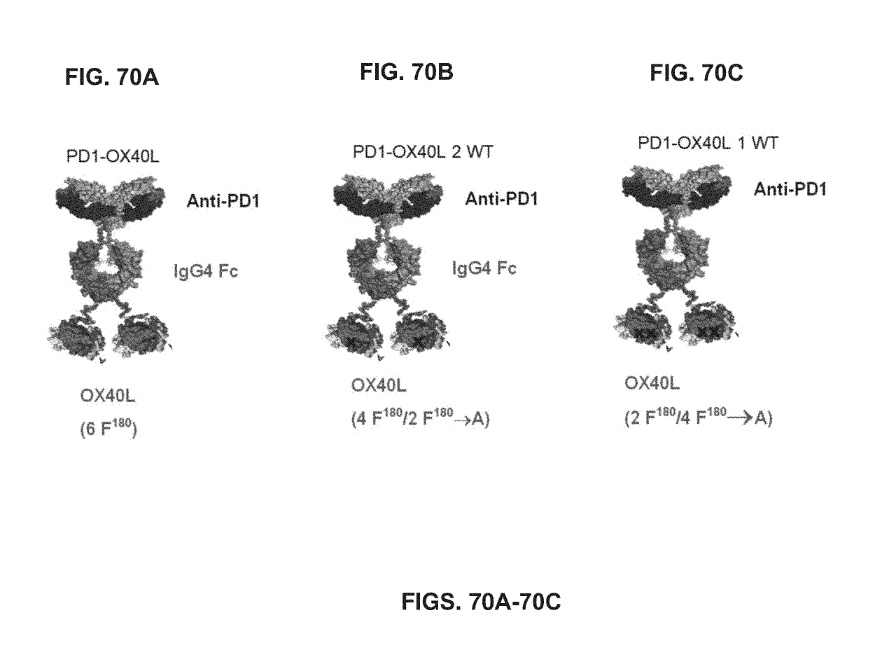

The studies described below show that an anti-PD-1-OX40 BFP can drive internalization. This is irrespective of whether the OX40 arm is an agonist or a non-agonist antibody. This finding is directly applicable to the autoimmune space. The results show that internalization of activating receptors can be driven using bispecifics built with non-agonist, but internalizing antibodies. See FIGS. 63-64 and 70-74. These results also show that degradation of PD-1 is independent of OX40 agonist function in this bispecific setting.

### Conclusions

MEDI7526 similar to CD40L FP, induced fast down-regulation of CD40. Interestingly, it was found that MEDI7526 also triggered rapid and robust down-regulation of PD-L1 on cell surface on multiple types of cells, including THP1, MDA-MB-231, and human monocytes. All of these cells express CD40. Moreover, MEDI7526 not only down-regulated PD-L1 on cell surface, but also significantly reduced total cellular amount of PD-L1 protein, suggesting forced internalization of PD-L1 may trigger its degradation. Moreover, anti-PD-L1-TNF-α BFP similarly triggered down-regulation of PD-L1 protein in T24 tumor cells. This result demonstrates that another BFP molecule which replaces CD40 with another TNF-α family protein can trigger cell surface PD-L1 downregulation. It is believed that additional BFP molecules can also similarly regulate PD-L1 down-regulation.

The data described herein from these novel, bispecific molecules, demonstrate that the combination of the two into one “scaffold” generates results that would be unachievable with combination therapy alone. MEDI7526 (BFP3), through its unique function of stimulating CD40 pathway and down-regulating PD-L1 expression, represents a promising therapeutic approach against cancer.

Similarly, anti-PD1-GITRL BFP (MEDI3387) and anti-PD1-OX40L BFP are shown to trigger degradation of PD1 protein in activated human PBMCs, which may provide another therapeutic option for fighting cancer.

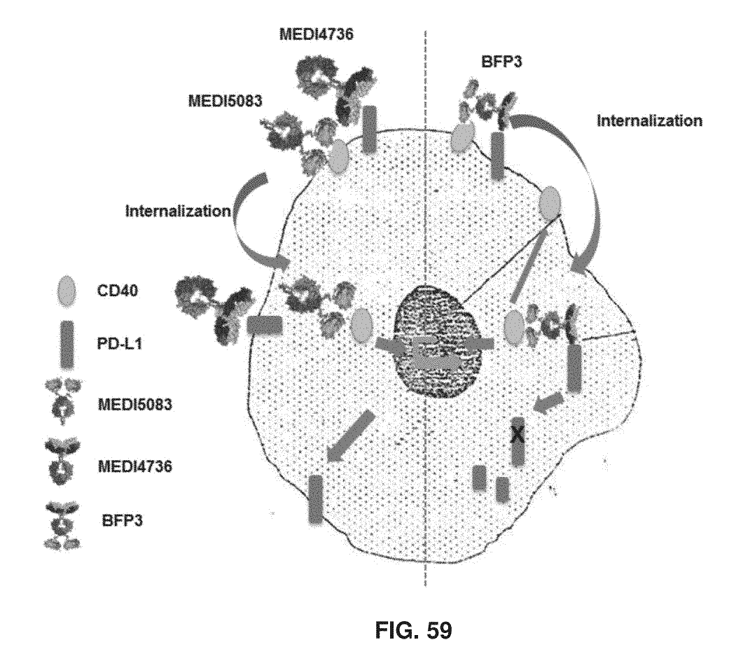

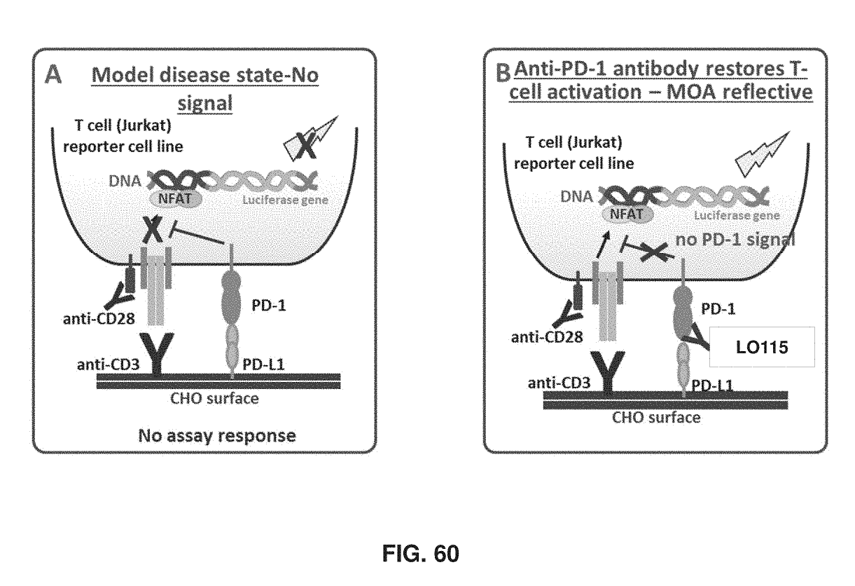

FIG. 59 illustrates the proposed MOA for MEDI7526 (BFP3). Specifically, by activating antigen presenting cells via CD40 ligation while simultaneously removing PD-L1 from the cell surface leads to the induction of IFN-γ, IL-12, and IL-10, but not TNF-α, or IL-6. The absence of induced TNF-α and IL-6 correlates with enhanced anti-tumor function and reduced weight loss, supporting the conclusion that MEDI7526 can induce enhanced anti-tumor responses with reduced toxicity.

Murine studies further confirm that MEDI7526 has the potential to treat liver tumors.

### Other Embodiments

From the foregoing description, it will be apparent that variations and modifications may be made to the disclosure described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

SEQUENCE ID NOS

| Sequence No. | Name |
|---|---|
| SEQ ID NO: 1 | α-PD-1 VH (heavy chain variable region) polypeptide |
| SEQ ID NO: 2 | α-PD-1 CH1 (heavy chain constant region 1) |
| | polypeptide |
| SEQ ID NO: 3 | α-PD-1 VL (light chain variable region) polypeptide |
| SEQ ID NO: 4 | α-PD-1 LC (light chain constant region) polypeptide |
| SEQ ID NO: 5 | α-PD-1 kappa light chain |
| SEQ ID NO: 6 | IgG1 Fc (hinge-CH2—CH3) polypeptide |
| SEQ ID NO: 7 | GITRL polypeptide |
| SEQ ID NO: 8 | BIOAE005/MEDI3387_HC (heavy chain) polypeptide |
| SEQ ID NO: 9 | IgG4 Fc (hinge_CH2_CH3) polypeptide |
| SEQ ID NO: 10 | BIOAE003/MEDI5771_HC (heavy chain) polypeptide |
| SEQ ID NO: 11 | BFP2-CD40L fusion protein (MEDI5083)-anti-PDL1 |
| | (MEDI4736 scFv)-G4P HC (no light chain b/c no |
| | Fab arms) |
| SEQ ID NO: 12 | BFP3-anti-PDL1 (MEDI4736)-CD40L fusion protein |
| | (MEDI5083)-G4P HC |
| SEQ ID NO: 13 | BFP3-anti-PDL1 (MEDI4736)-CD40L fusion protein |
| | (MEDI5083)-G4P LC (Same as LC of MEDI4736)/α- |
| | PD-L1 kappa light chain |
| SEQ ID NO: 14 | Murine BFP3-anti-muPDL1 (MEDI4736 Surrogate)- |
| | muCD40L fusion protein (MEDI5083 Surrogate)- |
| | muIgG1 D265A HC |
| SEQ ID NO: 15 | Murine BFP3-anti-muPDL1 (MEDI4736 Surrogate)- |
| | muCD40L fusion protein (MEDI5083 Surrogate)- |
| | muIgG1 D265A LC |
| SEQ ID NO: 16 | BFP3-anti-PDL1 (MEDI 4736)-TNF-α fusion |
| | protein-G4P HC |
| SEQ ID NO: 17 | BFP3-anti-PDL1 (MEDI 4736)-TNF-α fusion |
| | protein-G4P LC (same as LC of 4736)/α-PD-L1 |
| | kappa light chain |
| SEQ ID NO: 18 | BFP3-anti-PD1-OX40 ligand fusion protein-G4P HC |
| SEQ ID NO: 19 | BFP3-anti-PD1-OX40 ligand fusion protein-G4P LC |
| | (same as LC of LO115) |
| SEQ ID NO: 20 | CD40L sp\|P29965\|CD40L_HUMAN - Membrane |
| | bound form Cytoplasmic domain = 1-20; Signal |
| | anchor type II membrane protein region = 21-46; |
| | soluble form = 113-261 |
| SEQ ID NO: 21 | CD40L polypeptide - Soluble form |
| SEQ ID NO: 22 | CD40 polypeptide |
| SEQ ID NO: 23 | PD-L1 polypeptide |
| SEQ ID NO: 24 | Durvalumab VL polypeptide |
| SEQ ID NO: 25 | Durvalumab VH polypeptide |
| SEQ ID NO: 26 | Durvalumab VH CDR1 |
| SEQ ID NO: 27 | Durvalumab VH CDR2 |
| SEQ ID NO: 28 | Durvalumab VH CDR3 |
| SEQ ID NO: 29 | Durvalumab VL CDR1 |
| SEQ ID NO: 30 | Durvalumab VL CDR2 |
| SEQ ID NO: 31 | Durvalumab VL CDR3 |
| SEQ ID NO: 32 | PD-1 polypeptide |
| SEQ ID NO: 33 | 4x GGGGS repeat linker |
| SEQ ID NO: 34 | 3x GGGGS repeat linker |
| SEQ ID NO: 35 | 2x GGGGS repeat linker |
| SEQ ID NO: 36 | Variant linker |
| SEQ ID NO: 37 | BFP3-anti-PD1-OX40 1WT ligand fusion protein- |
| | G4P HC |
| SEQ ID NO: 38 | BFP3-anti-PD1-OX40 2WT ligand fusion protein- |
| | G4P HC |

SEQUENCES

| | |
|---|---|
| SEQ ID NO: 1 | EVQLVESGGGLVQPGGSLRLSCAASGFTFSD |
| | YGMHWVRQAPGKGLEWVAYISSGSYTIYSAD |
| | SVKGRFTISRDNAKNSLYLQMNSLRAEDTAV |
| | YYCARRAPNSFYEYYFDYWGQGTTVTVSS |
| SEQ ID NO: 2 | ASTKGPSVFPLAPSSKSTSGGTAALGCLVKD |
| | YFPEPVTVSWNSGALTSGVHTFPAVLQSSGL |
| | YSLSSVVTVPSSSLGTQTYICNVNHKPSNTK |
| | VDKRV |
| SEQ ID NO: 3 | QIVLTQSPATLSLSPGERATLSCSASSKHTN |
| | LYWSRHMYWYQQKPGQAPRLLIYLTSNRATG |
| | IPARFSGSGSGTDFTLTISSLEPEDFAVYYC |
| | QQWSSNPFTFGQGTKLEIK |
| SEQ ID NO: 4 | RTVAAPSVFIFPPSDEQLKSGTASVVCLLNN |
| | FYPREAKVQWKVDNALQSGNSQESVTEQDSK |
| | DSTYSLSSTLTLSKADYEKHKVYACEVTHQG |
| | LSSPVTKSFNRGEC |
| SEQ ID NO: 5 | QIVLTQSPATLSLSPGERATLSCSASSKHTN |
| | LYWSRHMYWYQQKPGQAPRLLIYLTSNRATG |
| | IPARFSGSGSGTDFTLTISSLEPEDFAVYYC |
| | QQWSSNPFTFGQGTKLEIKRTVAAPSVFIFP |
| | PSDEQLKSGTASVVCLLNNFYPREAKVQWKV |
| | DNALQSGNSQESVTEQDSKDSTYSLSSTLTL |
| | SKADYEKHKVYACEVTHQGLSSPVTKSFNRG |
| | EC |
| SEQ ID NO: 6 | EPKSCDKTHTCPPCPAPELLGGPSVFLFPPK |
| | PKDTLMISRTPEVTCVVVDVSHEDPEVKFNW |
| | YVDGVEVHNAKTKPREEQYNSTYRVVSVLTV |
| | LHQDWLNGKEYKCKVSNKALPAPIEKTISKA |
| | KGQPREPQVYTLPPSREEMTKNQVSLTCLVK |
| | GFYPSDIAVEWESNGQPENNYKTTPPVLDSD |
| | GSFFLYSKLTVDKSRWQQGNVFSCSVMHEAL |
| | HNHYTQKSLSLSPGK |
| SEQ ID NO: 7 | EPCMAKFGPLPSKWQMASSEPPCVNKVSDWK |
| | LEILQNGLYLIYGQVAPNANYNDVAPFEVRL |
| | YKNKDMIQTLTNKSKIQNVGGTYELHVGDTI |
| | DLIFNSEHQVLKDNTYWGIILLANPQFIS |
| SEQ ID NO: 8 | EVQLVESGGGLVQPGGSLRLSCAASGFTFSD |
| | YGMHWVRQAPGKGLEWVAYISSGSYTIYSAD |
| | SVKGRFTISRDNAKNSLYLQMNSLRAEDTAV |
| | YYCARRAPNSFYEYYFDYWGQGTTVTVSSAS |
| | TKGPSVFPLAPSSKSTSGGTAALGCLVKDYF |
| | PEPVTVSWNSGALTSGVHTFPAVLQSSGLYS |
| | LSSVVTVPSSSLGTQTYICNVNHKPSNTKVD |
| | KRVEPKSCDKTHTCPPCPAPELLGGPSVFLF |
| | PPKPKDTLMISRTPEVTCVVVDVSHEDPEVK |
| | FNWYVDGVEVHNAKTKPREEQYNSTYRVVSV |
| | LTVLHQDWLNGKEYKCKVSNKALPAPIEKTI |
| | SKAKGQPREPQVYTLPPSREEMTKNQVSLTC |
| | LVKGFYPSDIAVEWESNGQPENNYKTTPPVL |
| | DSDGSFFLYSKLTVDKSRWQQGNVFSCSVMH |
| | EALHNHYTQKSLSLSPGKGGGGSGGGGSGGG |
| | GSQLETAKEPCMAKFGPLPSKWQMASSEPPC |
| | VNKVSDWKLEILQNGLYLIYGQVAPNANYND |
| | VAPFEVRLYKNKDMIQTLTNKSKIQNVGGTY |
| | ELHVGDTIDLIFNSEHQVLKDNTYWGIILLA |
| | NPQFISGGGGSGGGGSEPCMAKFGPLPSKWQ |
| | MASSEPPCVNKVSDWKLEILQNGLYLIYGQV |
| | APNANYNDVAPFEVRLYKNKDMIQTLTNKSK |
| | IQNVGGTYELHVGDTIDLIFNSEHQVLKDNT |
| | YWGIILLANPQFISGGGGSGGGGSEPCMAKF |
| | GPLPSKWQMASSEPPCVNKVSDWKLEILQNG |
| | LYLIYGQVAPNANYNDVAPFEVRLYKNKDMI |
| | QTLTNKSKIQNVGGTYELHVGDTIDLIFNSE |
| | HQVLKDNTYWGIILLANPQFIS |
| SEQ ID NO: 9 | ESKYGPPCPPCPAPEFLGGPSVFLFPPKPKD |
| | TLMISRTPEVTCVVVDVSQEDPEVQFNWYVD |
| | GVEVHNAKTKPREEQFNSTYRVVSVLTVLHQ |
| | DWLNGKEYKCKVSNKGLPSSIEKTISKAKGQ |
| | PREPQVYTLPPSQEEMTKNQVSLTCLVKGFY |
| | PSDIAVEWESNGQPENNYKTTPPVLDSDGSF |
| | FLYSRLTVDKSRWQEGNVFSCSVMHEALHNH |
| | YTQKSLSLSLGK |
| SEQ ID NO: 10 | EVQLVESGGGLVQPGGSLRLSCAASGFTFSD |
| | YGMHWVRQAPGKGLEWVAYISSGSYTIYSAD |
| | SVKGRFTISRDNAKNSLYLQMNSLRAEDTAV |
| | YYCARRAPNSFYEYYFDYWGQGTTVTVSSAS |
| | TKGPSVFPLAPCSRSTSESTAALGCLVKDYF |
| | PEPVTVSWNSGALTSGVHTFPAVLQSSGLYS |
| | LSSVVTVPSSSLGTKTYTCNVDHKPSNTKVD |
| | KRVESKYGPPCPPCPAPEFLGGPSVFLFPPK |
| | PKDTLMISRTPEVTCVVVDVSQEDPEVQFNW |
| | YVDGVEVHNAKTKPREEQFNSTYRVVSVLTV |
| | LHQDWLNGKEYKCKVSNKGLPSSIEKTISKA |
| | KGQPREPQVYTLPPSQEEMTKNQVSLTCLVK |
| | GFYPSDIAVEWESNGQPENNYKTTPPVLDSD |
| | GSFFLYSRLTVDKSRWQEGNVFSCSVMHEAL |
| | HNHYTQKSLSLSLGKGGGGSGGGGSGGGGSQ |
| | LETAKEPCMAKFGPLPSKWQMASSEPPCVNK |
| | VSDWKLEILQNGLYLIYGQVAPNANYNDVAP |
| | FEVRLYKNKDMIQTLTNKSKIQNVGGTYELH |
| | VGDTIDLIFNSEHQVLKDNTYWGIILLANPQ |
| | FISGGGGSGGGGSEPCMAKFGPLPSKWQMAS |
| | SEPPCVNKVSDWKLEILQNGLYLIYGQVAPN |
| | ANYNDVAPFEVRLYKNKDMIQTLTNKSKIQN |
| | VGGTYELHVGDTIDLIFNSEHQVLKDNTYWG |
| | IILLANPQFISGGGGSGGGGSEPCMAKFGPL |
| | PSKWQMASSEPPCVNKVSDWKLEILQNGLYL |
| | IYGQVAPNANYNDVAPFEVRLYKNKDMIQTL |
| | TNKSKIQNVGGTYELHVGDTIDLIFNSEHQV |
| | LKDNTYWGIILLANPQFIS |
| SEQ ID NO: 11 | NPQIAAHVISEASSKTTSVLQWAEKGYYTMS |
| | NNLVTLENGKQLTVKRQGLYYIYAQVTFCSN |
| | REASSQAPFIASLWLKSPGRFERILLRAANT |
| | HSSAKPCGQQSIHLGGVFELQPGASVFVNVT |
| | DPSQVSHGTGFTSFGLLKLGGGGSGGGSQIA |
| | AHVISEASSKTTSVLQWAEKGYYTMSNNLVT |
| | LENGKQLTVKRQGLYYIYAQVTFCSNREASS |
| | QAPFIASLWLKSPGRFERILLRAANTHSSAK |
| | PCGQQSIHLGGVFELQPGASVFVNVTDPSQV |
| | SHGTGFTSFGLLKLGGGGSGGGSQIAAHVIS |
| | EASSKTTSVLQWAEKGYYTMSNNLVTLENGK |
| | QLTVKRQGLYYIYAQVTFCSNREASSQAPFI |
| | ASLWLKSPGRFERILLRAANTHSSAKPCGQQ |
| | SIHLGGVFELQPGASVFVNVTDPSQVSHGTG |
| | FTSFGLLKLGGGGSGGGGSGGGGSESKYGPP |
| | CPPCPAPEFLGGPSVFLFPPKPKDTLMISRT |
| | PEVTCVVVDVSQEDPEVQFNWYVDGVEVHNA |
| | KTKPREEQFNSTYRVVSVLTVLHQDWLNGKE |
| | YKCKVSNKGLPSSIEKTISKAKGQPREPQVY |
| | TLPPSQEEMTKNQVSLTCLVKGFYPSDIAVE |
| | WESNGQPENNYKTTPPVLDSDGSFFLYSRLT |
| | VDKSRWQEGNVFSCSVMHEALHNHYTQKSLS |
| | LSLGKGGGGSGGGGSEIVLTQSPGTLSLSPG |
| | ERATLSCRASQRVSSSYLAWYQQKPGQAPRL |
| | LIYDASSRATGIPDRFSGSGSGTDFTLTISR |
| | LEPEDFAVYYCQQYGSLPWTFGCGTKVEIKG |
| | GGGSGGGGSGGGGSGGGGSEVQLVESGGGLV |
| | QPGGSLRLSCAASGFTFSRYWMSWVRQAPGK |
| | CLEWVANIKQDGSEKYYVDSVKGRFTISRDN |
| | AKNSLYLQMNSLRAEDTAVYYCAREGGWFGE |
| | LAFDYWGQGTLVTVSS |
| SEQ ID NO: 12 | EVQLVESGGGLVQPGGSLRLSCAASGFTFSR |
| | YWMSWVRQAPGKGLEWVANIKQDGSEKYYVD |
| | SVKGRFTISRDNAKNSLYLQMNSLRAEDTAV |
| | YYCAREGGWFGELAFDYWGQGTLVTVSSAST |
| | KGPSVFPLAPCSRSTSESTAALGCLVKDYFP |
| | EPVTVSWNSGALTSGVHTFPAVLQSSGLYSL |
| | SSVVTVPSSSLGTKTYTCNVDHKPSNTKVDK |
| | RVESKYGPPCPPCPAPEFLGGPSVFLFPPKP |
| | KDTLMISRTPEVTCVVVDVSQEDPEVQFNWY |
| | VDGVEVHNAKTKPREEQFNSTYRVVSVLTVL |
| | HQDWLNGKEYKCKVSNKGLPSSIEKTISKAK |
| | GQPREPQVYTLPPSQEEMTKNQVSLTCLVKG |
| | FYPSDIAVEWESNGQPENNYKTTPPVLDSDG |
| | SFFLYSRLTVDKSRWQEGNVFSCSVMHEALH |
| | NHYTQKSLSLSLGKGGGGSGGGGSNPQIAAH |
| | VISEASSKTTSVLQWAEKGYYTMSNNLVTLE |
| | NGKQLTVKRQGLYYIYAQVTFCSNREASSQA |
| | PFIASLWLKSPGRFERILLRAANTHSSAKPC |
| | GQQSIHLGGVFELQPGASVFVNVTDPSQVSH |
| | GTGFTSFGLLKLGGGGSGGGSQIAAHVISEA |
| | SSKTTSVLQWAEKGYYTMSNNLVTLENGKQL |
| | TVKRQGLYYIYAQVTFCSNREASSQAPFIAS |
| | LWLKSPGRFERILLRAANTHSSAKPCGQQSI |
| | HLGGVFELQPGASVFVNVTDPSQVSHGTGFT |
| | SFGLLKLGGGGSGGGSQIAAHVISEASSKTT |
| | SVLQWAEKGYYTMSNNLVTLENGKQLTVKRQ |
| | GLYYIYAQVTFCSNREASSQAPFIASLWLKS |
| | PGRFERILLRAANTHSSAKPCGQQSIHLGGV |
| | FELQPGASVFVNVTDPSQVSHGTGFTSFGLL |
| | KL |
| SEQ ID NO: 13 | EIVLTQSPGTLSLSPGERATLSCRASQRVSS |
| | SYLAWYQQKPGQAPRLLIYDASSRATGIPDR |
| | FSGSGSGTDFTLTISRLEPEDFAVYYCQQYG |
| | SLPWTFGQGTKVEIKRTVAAPSVFIFPPSDE |
| | QLKSGTASVVCLLNNFYPREAKVQWKVDNAL |
| | QSGNSQESVTEQDSKDSTYSLSSTLTLSKAD |
| | YEKHKVYACEVTHQGLSSPVTKSFNRGEC |
| SEQ ID NO: 14 | QVKLLQSGAALVKPGDSLKMSCKASGYTFTD |
| | YLIHWVKQSHGKSLEWIGYINPYSGNTNYDE |
| | KFRSMATLTVDKSSSTAYMEFSRLTSEDSAI |
| | YYCARKDSSYIGGIWFAYWGQGTLVTVSSAS |
| | TTPPSVYPLAPGSAAQTNSMVTLGCLVKGYF |
| | PEPVTVTWNSGSLSSGVHTFPAVLQSDLYTL |
| | SSSVTVPSSTWPSETVTCNVAHPASSTKVDK |
| | KIVPRDCGCKPCICTVPEVSSVFIFPPKPKD |
| | VLTITLTPKVTCVVVAISKDDPEVQFSWFVD |
| | DVEVHTAQTQPREEQFNSTFRSVSELPIMHQ |
| | DWLNGKEFKCRVNSAAFPAPIEKTISKTKGR |
| | PKAPQVYTIPPPKEQMAKDKVSLTCMITDFF |
| | PEDITVEWQWNGQPAENYKNTQPIMDTDGSY |
| | FVYSKLNVQKSNWEAGNTFTCSVLHEGLHNH |
| | HTEKSLSHSPGKGGGGSGGGGSDPQIAAHVV |
| | SEANSNAASVLQWAKKGYYTMKSNLVMLENG |
| | KQLTVKREGLYYVYTQVTFCSNREPSSQRPF |
| | IVGLWLKPSSGSERILLKAANTHSSSQLCEQ |
| | QSVHLGGVFELQAGASVFVNVTEASQVIHRV |
| | GFSSFGLLKLGGGSGGSQIAAHVVSEANSNA |
| | ASVLQWAKKGYYTMKSNLVMLENGKQLTVKR |
| | EGLYYVYTQVTFCSNREPSSQRPFIVGLWLK |
| | PSSGSERILLKAANTHSSSQLCEQQSVHLGG |
| | VFELQAGASVFVNVTEASQVIHRVGFSSFGL |
| | LKLGGGSGGSQIAAHVVSEANSNAASVLQWA |
| | KKGYYTMKSNLVMLENGKQLTVKREGLYYVY |
| | TQVTFCSNREPSSQRPFIVGLWLKPSSGSER |
| | ILLKAANTHSSSQLCEQQSVHLGGVFELQAG |
| | ASVFVNVTEASQVIHRVGFSSFGLLKL |
| SEQ ID NO: 15 | GDTVLTQSPALAVSPGERVTISCWASESVST |
| | LTHWYQQKPGQQPKLLIYLASHLESGVPARF |
| | SGSGSGTDFTLTIDPVEADDTATYYCHQTWN |
| | NPPTFGAGTKLELTRADAAPTVSIFPPSSEQ |
| | LTSGGASVVCFLNNFYPKDINVKWKIDGSER |
| | QNGVLNSWTDQDSKDSTYSMSSTLTLTKDEY |
| | ERHNSYTCEATHKTSTSPIVKSFNRNEC |
| SEQ ID NO: 16 | EVQLVESGGGLVQPGGSLRLSCAASGFTFSR |
| | YWMSWVRQAPGKGLEWVANIKQDGSEKYYVD |
| | SVKGRFTISRDNAKNSLYLQMNSLRAEDTAV |
| | YYCAREGGWFGELAFDYWGQGTLVTVSSAST |
| | KGPSVFPLAPCSRSTSESTAALGCLVKDYFP |
| | EPVTVSWNSGALTSGVHTFPAVLQSSGLYSL |
| | SSVVTVPSSSLGTKTYTCNVDHKPSNTKVDK |
| | RVESKYGPPCPPCPAPEFLGGPSVFLFPPKP |
| | KDTLMISRTPEVTCVVVDVSQEDPEVQFNWY |
| | VDGVEVHNAKTKPREEQFNSTYRVVSVLTVL |
| | HQDWLNGKEYKCKVSNKGLPSSIEKTISKAK |
| | GQPREPQVYTLPPSQEEMTKNQVSLTCLVKG |
| | FYPSDIAVEWESNGQPENNYKTTPPVLDSDG |
| | SFFLYSRLTVDKSRWQEGNVFSCSVMHEALH |
| | NHYTQKSLSLSLGKGGGGSGGGGSSDKPVAH |
| | VVANPQAEGQLQWLNRRANALLANGVELRDN |
| | QLVVPSEGLYLIYSQVLFKGQGCPSTHVLLT |
| | HTISRIAVSYQTKVNLLSAIKSPCQRETPEG |
| | AEAKPWYEPIYLGGVFQLEKGDRLSAEINRP |
| | DYLDFAESGQVYFGIIALGGGGSGGGGSGGG |
| | GSSDKPVAHVVANPQAEGQLQWLNRRANALL |
| | ANGVELRDNQLVVPSEGLYLIYSQVLFKGQG |
| | CPSTHVLLTHTISRIAVSYQTKVNLLSAIKS |
| | PCQRETPEGAEAKPWYEPIYLGGVFQLEKGD |
| | RLSAEINRPDYLDFAESGQVYFGIIALGGGG |
| | SGGGGSGGGGSSDKPVAHVVANPQAEGQLQW |
| | LNRRANALLANGVELRDNQLVVPSEGLYLIY |
| | SQVLFKGQGCPSTHVLLTHTISRIAVSYQTK |
| | VNLLSAIKSPCQRETPEGAEAKPWYEPIYLG |
| | GVFQLEKGDRLSAEINRPDYLDFAESGQVYF |
| | GIIAL |
| SEQ ID NO: 17 | EIVLTQSPGTLSLSPGERATLSCRASQRVSS |
| | SYLAWYQQKPGQAPRLLIYDASSRATGIPDR |
| | FSGSGSGTDFTLTISRLEPEDFAVYYCQQYG |
| | SLPWTFGQGTKVEIKRTVAAPSVFIFPPSDE |
| | QLKSGTASVVCLLNNFYPREAKVQWKVDNAL |
| | QSGNSQESVTEQDSKDSTYSLSSTLTLSKAD |
| | YEKHKVYACEVTHQGLSSPVTKSFNRGEC |
| SEQ ID NO: 18 | EVQLVESGGGLVQPGGSLRLSCAASGFTFSD |
| | YGMHWVRQAPGKGLEWVAYISSGSYTIYSAD |
| | SVKGRFTISRDNAKNSLYLQMNSLRAEDTAV |
| | YYCARRAPNSFYEYYFDYWGQGTTVTVSSAS |
| | TKGPSVFPLAPCSRSTSESTAALGCLVKDYF |
| | PEPVTVSWNSGALTSGVHTFPAVLQSSGLYS |
| | LSSVVTVPSSSLGTKTYTCNVDHKPSNTKVD |
| | KRVESKYGPPCPPCPAPEFLGGPSVFLFPPK |
| | PKDTLMISRTPEVTCVVVDVSQEDPEVQFNW |
| | YVDGVEVHNAKTKPREEQFNSTYRVVSVLTV |
| | LHQDWLNGKEYKCKVSNKGLPSSIEKTISKA |
| | KGQPREPQVYTLPPSQEEMTKNQVSLTCLVK |
| | GFYPSDIAVEWESNGQPENNYKTTPPVLDSD |
| | GSFFLYSRLTVDKSRWQEGNVFSCSVMHEAL |
| | HNHYTQKSLSLSLGKGGGGSGGGGSGGGGSQ |
| | VSHRYPRIQSIKVQFTEYKKEKGFILTSQKE |
| | DEIMKVQNNSVIINCDGFYLISLKGYFSQEV |
| | NISLHYQKDEEPLFQLKKVRSVNSLMVASLT |
| | YKDKVYLNVTTDNTSLDDFHVNGGELILIHQ |
| | NPGEFCVLGGGGSGGGGSQVSHRYPRIQSIK |
| | VQFTEYKKEKGFILTSQKEDEIMKVQNNSVI |
| | INCDGFYLISLKGYFSQEVNISLHYQKDEEP |
| | LFQLKKVRSVNSLMVASLTYKDKVYLNVTTD |
| | NTSLDDFHVNGGELILIHQNPGEFCVLGGGG |
| | SGGGGSQVSHRYPRIQSIKVQFTEYKKEKGF |
| | ILTSQKEDEIMKVQNNSVIINCDGFYLISLK |
| | GYFSQEVNISLHYQKDEEPLFQLKKVRSVNS |
| | LMVASLTYKDKVYLNVTTDNTSLDDFHVNGG |
| | ELILIHQNPGEFCVL |
| SEQ ID NO: 19 | QIVLTQSPATLSLSPGERATLSCSASSKHTN |
| | LYWSRHMYWYQQKPGQAPRLLIYLTSNRATG |
| | IPARFSGSGSGTDFTLTISSLEPEDFAVYYC |
| | QQWSSNPFTFGQGTKLEIKRTVAAPSVFIFP |
| | PSDEQLKSGTASVVCLLNNFYPREAKVQWKV |
| | DNALQSGNSQESVTEQDSKDSTYSLSSTLTL |
| | SKADYEKHKVYACEVTHQGLSSPVTKSFNRG |
| | EC |
| SEQ ID NO: 20 | MIETYNQTSPRSAATGLPISMKIFMYLLTVF |
| | LITQMIGSALFAVYLHRRLDKIEDERNLHED |
| | FVFMKTIQRCNTGERSLSLLNCEEIKSQFEG |
| | FVKDIMLNKEETKKENSFEMQKGDQNPQIAA |
| | HVISEASSKTTSVLQWAEKGYYTMSNNLVTL |
| | ENGKQLTVKRQGLYYIYAQVTFCSNREASSQ |
| | APFIASLCLKSPGRFERILLRAANTHSSAKP |
| | CGQQSIHLGGVFELQPGASVFVNVTDPSQVS |
| | HGTGFTSFGLLKL |
| SEQ ID NO: 21 | MQKGDQNPQIAAHVISEASSKTTSVLQWAEK |
| | GYYTMSNNLVTLENGKQLTVKRQGLYYIYAQ |
| | VTFCSNREASSQAPFIASLCLKSPGRFERIL |
| | LRAANTHSSAKPCGQQSIHLGGVFELQPGAS |
| | VFVNVTDPSQVSHGTGFTSFGLLKL |
| SEQ ID NO: 22 | MVRLPLQCVLWGCLLTAVHPEPPTACREKQY |
| | LINSQCCSLCQPGQKLVSDCTEFTETECLPC |
| | GESEFLDTWNRETHCHQHKYCDPNLGLRVQQ |
| | KGTSETDTICTCEEGWHCTSEACESCVLHRS |
| | CSPGFGVKQIATGVSDTICEPCPVGFFSNVS |
| | SAFEKCHPWTSCETKDLVVQQAGTNKTDVVC |
| | GPQDRLRALVVIPIIFGILFAILLVLVFIKK |
| | VAKKPTNKAPHPKQEPQEINFPDDLPGSNTA |
| | APVQETLHGCQPVTQEDGKESRISVQERQ |
| SEQ ID NO: 23 | MRIFAVFIFMTYWHLLNAPYNKINQRILVVD |
| | PVTSEHELTCQAEGYPKAEVIWTSSDHQVLS |
| | GKTTTTNSKREEKLFNVTSTLRINTTTNEIF |
| | YCTFRRLDPEENHTAELVIPELPLAHPPNER |
| | THLVILGAILLCLGVALTFIFRLRKGRMMDV |
| | KKCGIQDTNSKKQSDTHLEET |
| SEQ ID NO: 24 | EIVLTQSPGTLSLSPGERATLSCRASQRVSS |
| | SYLAWYQQKPGQAPRLLIYDASSRATGIPDR |
| | FSGSGSGTDFTLTISRLEPEDFAVYYCQQYG |
| | SLPWTFGQGTKVEIK |
| SEQ ID NO: 25 | EVQLVESGGGLVQPGGSLRLSCAASGFTFSR |
| | YWMSWVRQAPGKGLEWVANIKQDGSEKYYVD |
| | SVKGRFTISRDNAKNSLYLQMNSLRAEDTAV |
| | YYCAREGGWFGELAFDYWGQGTLVTVSS |
| SEQ ID NO: 26 | GFTFSRYWMS |
| SEQ ID NO: 27 | NIKQDGSEKYYVDSVKG |
| SEQ ID NO: 28 | EGGWFGELAFDY |
| SEQ ID NO: 29 | RASQRVSSSYLA |
| SEQ ID NO: 30 | DASSRAT |
| SEQ ID NO: 31 | QQYGSLPWT |
| SEQ ID NO: 32 | MQIPQAPWPVVWAVLQLGWRPGWFLDSPDRP |
| | WNPPTFSPALLVVTEGDNATFTCSFSNTSES |
| | FVLNWYRMSPSNQTDKLAAFPEDRSQPGQDC |
| | RFRVTQLPNGRDFHMSVVRARRNDSGTYLCG |
| | AISLAPKAQIKESLRAELRVTERRAEVPTAH |
| | PSPSPRPAGQFQTLVVGVVGGLLGSLVLLVW |
| | VLAVICSRAARGTIGARRTGQPLKEDPSAVP |
| | VFSVDYGELDFQWREKTPEPPVPCVPEQTEY |
| | ATIVFPSGMGTSSPARRGSADGPRSAQPLRP |
| | EDGHCSWPL |
| SEQ ID NO: 33 | GGGGSGGGGSGGGGSGGGGS |
| SEQ ID NO: 34 | GGGGSGGGGSGGGGS |
| SEQ ID NO: 35 | GGGGSGGGGS |
| SEQ ID NO: 36 | GGGGSGGGS |
| SEQ ID NO: 37 | EVQLVESGGGLVQPGGSLRLSCAASGFTFSD |
| | YGMHWVRQAPGKGLEWVAYISSGSYTIYSAD |
| | SVKGRFTISRDNAKNSLYLQMNSLRAEDTAV |
| | YYCARRAPNSFYEYYFDYWGQGTTVTVSSAS |
| | TKGPSVFPLAPCSRSTSESTAALGCLVKDYF |
| | PEPVTVSWNSGALTSGVHTFPAVLQSSGLYS |
| | LSSVVTVPSSSLGTKTYTCNVDHKPSNTKVD |
| | KRVESKYGPPCPPCPAPEFLGGPSVFLFPPK |
| | PKDTLMISRTPEVTCVVVDVSQEDPEVQFNW |
| | YVDGVEVHNAKTKPREEQFNSTYRVVSVLTV |
| | LHQDWLNGKEYKCKVSNKGLPSSIEKTISKA |
| | KGQPREPQVYTLPPSQEEMTKNQVSLTCLVK |
| | GFYPSDIAVEWESNGQPENNYKTTPPVLDSD |
| | GSFFLYSRLTVDKSRWQEGNVFSCSVMHEAL |
| | HNHYTQKSLSLSLGKGGGGSGGGGSGGGGSQ |
| | VSHRYPRIQSIKVQFTEYKKEKGFILTSQKE |
| | DEIMKVQNNSVIINCDGFYLISLKGYFSQEV |
| | NISLHYQKDEEPLFQLKKVRSVNSLMVASLT |
| | YKDKVYLNVTTDNTSLDDFHVNGGELILIHQ |
| | NPGEACVLGGGGSGGGGSQVSHRYPRIQSIK |
| | VQFTEYKKEKGFILTSQKEDEIMKVQNNSVI |
| | INCDGFYLISLKGYFSQEVNISLHYQKDEEP |
| | LFQLKKVRSVNSLMVASLTYKDKVYLNVTTD |
| | NTSLDDFHVNGGELILIHQNPGEACVLGGGG |
| | SGGGGSQVSHRYPRIQSIKVQFTEYKKEKGF |
| | ILTSQKEDEIMKVQNNSVIINCDGFYLISLK |
| | GYFSQEVNISLHYQKDEEPLFQLKKVRSVNS |
| | LMVASLTYKDKVYLNVTTDNTSLDDFHVNGG |
| | ELILIHQNPGEFCVL |
| SEQ ID NO: 38 | EVQLVESGGGLVQPGGSLRLSCAASGFTFSD |
| | YGMHWVRQAPGKGLEWVAYISSGSYTIYSAD |
| | SVKGRFTISRDNAKNSLYLQMNSLRAEDTAV |
| | YYCARRAPNSFYEYYFDYWGQGTTVTVSSAS |
| | TKGPSVFPLAPCSRSTSESTAALGCLVKDYF |
| | PEPVTVSWNSGALTSGVHTFPAVLQSSGLYS |
| | LSSVVTVPSSSLGTKTYTCNVDHKPSNTKVD |
| | KRVESKYGPPCPPCPAPEFLGGPSVFLFPPK |
| | PKDTLMISRTPEVTCVVVDVSQEDPEVQFNW |
| | YVDGVEVHNAKTKPREEQFNSTYRVVSVLTV |
| | LHQDWLNGKEYKCKVSNKGLPSSIEKTISKA |
| | KGQPREPQVYTLPPSQEEMTKNQVSLTCLVK |
| | GFYPSDIAVEWESNGQPENNYKTTPPVLDSD |
| | GSFFLYSRLTVDKSRWQEGNVFSCSVMHEAL |
| | HNHYTQKSLSLSLGKGGGGSGGGGSGGGGSQ |
| | VSHRYPRIQSIKVQFTEYKKEKGFILTSQKE |
| | DEIMKVQNNSVIINCDGFYLISLKGYFSQEV |
| | NISLHYQKDEEPLFQLKKVRSVNSLMVASLT |
| | YKDKVYLNVTTDNTSLDDFHVNGGELILIHQ |
| | NPGEACVLGGGGSGGGGSQVSHRYPRIQSIK |
| | VQFTEYKKEKGFILTSQKEDEIMKVQNNSVI |
| | INCDGFYLISLKGYFSQEVNISLHYQKDEEP |
| | LFQLKKVRSVNSLMVASLTYKDKVYLNVTTD |
| | NTSLDDFHVNGGELILIHQNPGEFCVLGGGG |
| | SGGGGSQVSHRYPRIQSIKVQFTEYKKEKGF |
| | ILTSQKEDEIMKVQNNSVIINCDGFYLISLK |
| | GYFSQEVNISLHYQKDEEPLFQLKKVRSVNS |
| | LMVASLTYKDKVYLNVTTDNTSLDDFHVNGG |
| | ELILIHQNPGEFCVL |

1. A bispecific fusion protein, comprising:
a single chain fusion protein comprising a first binding region specific for a first cell surface target, an Fc monomer, and a second binding region specific for a second cell surface target,
wherein the first binding region and the second binding region are covalently linked to the Fc monomer via a peptide linker, and
wherein the bispecific fusion protein is capable of binding the first cell surface target and the second cell surface target at the same time.

2. The bispecific fusion protein of claim 1, wherein at least one of the first binding region and the second binding region is a Fab fragment or a receptor ligand.

3. The bispecific protein of claim 2, wherein the Fab fragment is an anti-PD-1 Fab fragment.

4. The bispecific fusion protein of claim 2, wherein the Fab fragment is an anti-PD-L1 Fab fragment.

5. The bispecific fusion protein according to claim 1, wherein the Fc monomer is an IgG1 Fc monomer.

6. The bispecific fusion protein of claim 5, wherein the IgG1 Fc monomer comprises an amino acid sequence having at least about 85% amino acid sequence identity to SEQ ID NO: 6.

7. The bispecific fusion protein according to claim 1, wherein the Fc monomer is an IgG4 Fc monomer.

8. The bispecific fusion protein of claim 7, wherein the IgG4 Fc monomer comprises an amino acid sequence having at least about 85% amino acid sequence identity to SEQ ID NO: 9.

9. The bispecific fusion protein of claim 1, wherein the Fc monomer comprises a hinge region.

10. The bispecific fusion protein of claim 1, wherein the Fc monomer comprises a human Fc amino acid sequence.

11. The bispecific fusion protein of claim 1, wherein at least one of the one or more ligand subunits is GITRL.

12. The bispecific fusion protein of claim 1, wherein at least one of the one or more ligand subunits is OX40L.

13. The bispecific fusion protein of claim 1, wherein at least one of the one or more ligand subunits is CD40L.

14. The bispecific fusion protein of claim 13, wherein the CD40L ligand subunit comprises a Trp residue at position 194.

15. The bispecific fusion protein of claim 14, wherein the Trp residue at position 194 is a C→W substitution.

16. The bispecific fusion protein of claim 1, wherein at least one of the one or more ligand subunits is TNF-α.

17. The bispecific fusion protein of claim 1, wherein at least one of the one or more ligand subunits is CD137L.

18. The bispecific fusion protein of claim 1, wherein the Fab fragment is linked to the N-terminus of the Fc monomer.

19. The bispecific fusion protein of claim 18, wherein the one or more ligand subunits is linked to the C-terminus of the Fc monomer.

20. The bispecific fusion protein of claim 1, wherein the Fab fragment is linked to the C-terminus of the Fc monomer.

21. The bispecific fusion protein of claim 20, wherein the one or more ligand subunits is linked to the N-terminus of the Fc monomer.

22. The bispecific fusion protein of claim 1, wherein the single chain fusion protein comprises a plurality of ligand subunits.

23. The bispecific fusion protein of claim 22, wherein the plurality of ligand subunits comprises 2, 3, 4, 5, 6, 7, 8, 9, or 10 ligand subunits.

24. The bispecific fusion protein of claim 1, wherein the single chain fusion protein comprises 3 ligand subunits.

25. The bispecific fusion protein of claim 24, wherein the ligand subunits form a homotrimer with 3 ligand subunits linked serially from N-terminus to C-terminus.

26. The bispecific fusion protein of claim 1, wherein the peptide linker comprises about 9 to about 20 amino acids.

27. The bispecific fusion protein of claim 26, wherein the peptide linker comprises about 9 to about 15 amino acids.

28. The bispecific fusion protein of claim 27, wherein the peptide linker comprises about 9 amino acids.

29. The bispecific fusion protein of claim 1, wherein the peptide linker comprises one or more glycine (Gly) or serine (Ser) residues.

30. The bispecific fusion protein of claim 1, wherein the peptide linker is GGGGSGGGGSGGGGSGGGGS (SEQ ID NO: 39), GGGGSGGGGSGGGGS (SEQ ID NO: 40), GGGGSGGGGS (SEQ ID NO:
35), or GGGGSGGGS (SEQ IN NO: 41).

31. A dimer comprising two bispecific fusion proteins selected from the bispecific fusion proteins of claim 1, wherein the dimer is formed via interaction of the Fc monomers.

32. A method of enhancing an anti-tumor immune response in a subject comprising administering to the subject the isolated fusion protein or dimer of claim 1.

33. The method of claim 32, wherein the subject has cancer.

34. The method of claim 33, wherein the method enhances one or more of an immune response and/or an anti-cancer response.

35. The method of claim 34, wherein the method results in a reduced toxicity compared to treatment with parental reagents of the isolated fusion protein or dimer.

36. A method of treating cancer comprising treating a patient in need thereof with the bispecific fusion protein of claim 13.

37. The method of claim 36 further comprising treatment with chemotherapy.

38. The method of claim 36, wherein the cancer is liver cancer.


SEQUENCE LISTING (43 sequences)

SEQ ID NO: 1 - protein, 122 aa, Artificial Sequence
  Description of Artificial Sequence Synthetic polypeptide
EVQLVESGGGLVQPGGSLRLSCAASGFTFSDYGMHWVRQAPGKGLEWVAYISSGSYTIYSADSVKGRFTISRDNAKNSLYLQMNSLRAEDTAVYYCARRAPNSFYEYYFDYWGQGTTVTVSS

SEQ ID NO: 2 - protein, 98 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 3 - protein, 112 aa, Artificial Sequence
  Description of Artificial Sequence Synthetic polypeptide
QIVLTQSPATLSLSPGERATLSCSASSKHTNLYWSRHMYWYQQKPGQAPRLLIYLTSNRATGIPARFSGSGSGTDFTLTISSLEPEDFAVYYCQQWSSNPFTFGQGTKLEIK

SEQ ID NO: 4 - protein, 107 aa, Artificial Sequence [sequence omitted]

SEQ ID NO: 5 - protein, 219 aa, Artificial Sequence
  Description of Artificial Sequence Synthetic polypeptide
QIVLTQSPATLSLSPGERATLSCSASSKHTNLYWSRHMYWYQQKPGQAPRLLIYLTSNRATGIPARFSGSGSGTDFTLTISSLEPEDFAVYYCQQWSSNPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 232 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 122 aa, Artificial Sequence
  Description of Artificial Sequence Synthetic polypeptide
EPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKDNTYWGIILLANPQFIS

SEQ ID NO: 8 - protein, 859 aa, Artificial Sequence
  Description of Artificial Sequence Synthetic polypeptide
EVQLVESGGGLVQPGGSLRLSCAASGFTFSDYGMHWVRQAPGKGLEWVAYISSGSYTIYSADSVKGRFTISRDNAKNSLYLQMNSLRAEDTAVYYCARRAPNSFYEYYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQLETAKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKDNTYWGIILLANPQFISGGGGSGGGGSEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKDNTYWGIILLANPQFISGGGGSGGGGSEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKDNTYWGIILLANPQFIS

SEQ ID NO: 9 - protein, 229 aa, Homo sapiens
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 856 aa, Artificial Sequence
  Description of Artificial Sequence Synthetic polypeptide
EVQLVESGGGLVQPGGSLRLSCAASGFTFSDYGMHWVRQAPGKGLEWVAYISSGSYTIYSADSVKGRFTISRDNAKNSLYLQMNSLRAEDTAVYYCARRAPNSFYEYYFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSQLETAKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKDNTYWGIILLANPQFISGGGGSGGGGSEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKDNTYWGIILLANPQFISGGGGSGGGGSEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKDNTYWGIILLANPQFIS

SEQ ID NO: 11 - protein, 946 aa, Artificial Sequence
  Description of Artificial Sequence Synthetic polypeptide
NPQIAAHVISEASSKTTSVLQWAEKGYYTMSNNLVTLENGKQLTVKRQGLYYIYAQVTFCSNREASSQAPFIASLWLKSPGRFERILLRAANTHSSAKPCGQQSIHLGGVFELQPGASVFVNVTDPSQVSHGTGFTSFGLLKLGGGGSGGGSQIAAHVISEASSKTTSVLQWAEKGYYTMSNNLVTLENGKQLTVKRQGLYYIYAQVTFCSNREASSQAPFIASLWLKSPGRFERILLRAANTHSSAKPCGQQSIHLGGVFELQPGASVFVNVTDPSQVSHGTGFTSFGLLKLGGGGSGGGSQIAAHVISEASSKTTSVLQWAEKGYYTMSNNLVTLENGKQLTVKRQGLYYIYAQVTFCSNREASSQAPFIASLWLKSPGRFERILLRAANTHSSAKPCGQQSIHLGGVFELQPGASVFVNVTDPSQVSHGTGFTSFGLLKLGGGGSGGGGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSEIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGCGTKVEIKGGGGSGGGGSGGGGSGGGGSEVQLVESGGGLVQPGGSLRLSCAASGFTFSRYWMSWVRQAPGKCLEWVANIKQDGSEKYYVDSVKGRFTISRDNAKNSLYLQMNSLRAEDTAVYYCAREGGWFGELAFDYWGQGTLVTVSS

SEQ ID NO: 12 - protein, 901 aa, Artificial Sequence
  Description of Artificial Sequence Synthetic polypeptide
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSNPQIAAHVISEASSKTTSVLQWAEKGYYTMSNNLVTLENGKQLTVKRQGLYYIYAQVTFCSNREASSQAPFIASLWLKSPGRFERILLRAANTHSSAKPCGQQSIHLGGVFELQPGASVFVNVTDPSQVSHGTGFTSFGLLKLGGGGSGGGSQIAAHVISEASSKTTSVLQWAEKGYYTMSNNLVTLENGKQLTVKRQGLYYIYAQVTFCSNREASSQAPFIASLWLKSPGRFERILLRAANTHSSAKPCGQQSIHLGGVFELQPGASVFVNVTDPSQVSHGTGFTSFGLLKLGGGGSGGGSQIAAHVISEASSKTTSVLQWAEKGYYTMSNNLVTLENGKQLTVKRQGLYYIYAQVTFCSNREASSQAPFIASLWLKSPGRFERILLRAANTHSSAKPCGQQSIHLGGVFELQPGASVFVNVTDPSQVSHGTGFTSFGLLKL

SEQ ID NO: 13 - protein, 215 aa, Artificial Sequence
  Description of Artificial Sequence Synthetic polypeptide
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 895 aa, Artificial Sequence
  Description of Artificial Sequence Synthetic polypeptide
QVKLLQSGAALVKPGDSLKMSCKASGYTFTDYLIHWVKQSHGKSLEWIGYINPYSGNTNYDEKFRSMATLTVDKSSSTAYMEFSRLTSEDSAIYYCARKDSSYIGGIWFAYWGQGTLVTVSSASTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGKGGGGSGGGGSDPQIAAHVVSEANSNAASVLQWAKKGYYTMKSNLVMLENGKQLTVKREGLYYVYTQVTFCSNREPSSQRPFIVGLWLKPSSGSERILLKAANTHSSSQLCEQQSVHLGGVFELQAGASVFVNVTEASQVIHRVGFSSFGLLKLGGGSGGSQIAAHVVSEANSNAASVLQWAKKGYYTMKSNLVMLENGKQLTVKREGLYYVYTQVTFCSNREPSSQRPFIVGLWLKPSSGSERILLKAANTHSSSQLCEQQSVHLGGVFELQAGASVFVNVTEASQVIHRVGFSSFGLLKLGGGSGGSQIAAHVVSEANSNAASVLQWAKKGYYTMKSNLVMLENGKQLTVKREGLYYVYTQVTFCSNREPSSQRPFIVGLWLKPSSGSERILLKAANTHSSSQLCEQQSVHLGGVFELQAGASVFVNVTEASQVIHRVGFSSFGLLKL

SEQ ID NO: 15 - protein, 214 aa, Artificial Sequence
  Description of Artificial Sequence Synthetic polypeptide
GDTVLTQSPALAVSPGERVTISCWASESVSTLTHWYQQKPGQQPKLLIYLASHLESGVPARFSGSGSGTDFTLTIDPVEADDTATYYCHQTWNNPPTFGAGTKLELTRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 16 - protein, 935 aa, Artificial Sequence
  Description of Artificial Sequence Synthetic polypeptide
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIALGGGGSGGGGSGGGGSSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIALGGGGSGGGGSGGGGSSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 17 - protein, 215 aa, Artificial Sequence; identical to SEQ ID NO: 13
  Description of Artificial Sequence Synthetic polypeptide

SEQ ID NO: 18 - protein, 883 aa, Artificial Sequence
  Description of Artificial Sequence Synthetic polypeptide
EVQLVESGGGLVQPGGSLRLSCAASGFTFSDYGMHWVRQAPGKGLEWVAYISSGSYTIYSADSVKGRFTISRDNAKNSLYLQMNSLRAEDTAVYYCARRAPNSFYEYYFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGGGGSGGGGSQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGGGGSGGGGSQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 19 - protein, 219 aa, Artificial Sequence; identical to SEQ ID NO: 5
  Description of Artificial Sequence Synthetic polypeptide

SEQ ID NO: 20 - protein, 261 aa, Homo sapiens
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 21 - protein, 149 aa, Homo sapiens
MQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 22 - protein, 277 aa, Homo sapiens
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

SEQ ID NO: 23 - protein, 176 aa, Homo sapiens
MRIFAVFIFMTYWHLLNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 24 - protein, 108 aa, Artificial Sequence
  Description of Artificial Sequence Synthetic polypeptide
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIK

SEQ ID NO: 25 - protein, 121 aa, Artificial Sequence
  Description of Artificial Sequence Synthetic polypeptide
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSS

SEQ ID NO: 26 - protein, 10 aa, Artificial Sequence
  Description of Artificial Sequence Synthetic peptide
GFTFSRYWMS

SEQ ID NO: 27 - protein, 17 aa, Artificial Sequence
  Description of Artificial Sequence Synthetic peptide
NIKQDGSEKYYVDSVKG

SEQ ID NO: 28 - protein, 12 aa, Artificial Sequence
  Description of Artificial Sequence Synthetic peptide
EGGWFGELAFDY

SEQ ID NO: 29 - protein, 12 aa, Artificial Sequence
  Description of Artificial Sequence Synthetic peptide
RASQRVSSSYLA

SEQ ID NO: 30 - protein, 7 aa, Artificial Sequence
  Description of Artificial Sequence Synthetic peptide
DASSRAT

SEQ ID NO: 31 - protein, 9 aa, Artificial Sequence
  Description of Artificial Sequence Synthetic peptide
QQYGSLPWT

SEQ ID NO: 32 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 33 - protein, 20 aa, Artificial Sequence
  Description of Artificial Sequence Synthetic peptide
GGGGSGGGGSGGGGSGGGGS

SEQ ID NO: 34 - protein, 15 aa, Artificial Sequence
  Description of Artificial Sequence Synthetic peptide
GGGGSGGGGSGGGGS

SEQ ID NO: 35 - protein, 10 aa, Artificial Sequence
  Description of Artificial Sequence Synthetic peptide
GGGGSGGGGS

SEQ ID NO: 36 - protein, 9 aa, Artificial Sequence
  Description of Artificial Sequence Synthetic peptide
GGGGSGGGS

SEQ ID NO: 37 - protein, 883 aa, Artificial Sequence
  Description of Artificial Sequence Synthetic polypeptide
EVQLVESGGGLVQPGGSLRLSCAASGFTFSDYGMHWVRQAPGKGLEWVAYISSGSYTIYSADSVKGRFTISRDNAKNSLYLQMNSLRAEDTAVYYCARRAPNSFYEYYFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEACVLGGGGSGGGGSQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEACVLGGGGSGGGGSQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 38 - protein, 883 aa, Artificial Sequence
  Description of Artificial Sequence Synthetic polypeptide
EVQLVESGGGLVQPGGSLRLSCAASGFTFSDYGMHWVRQAPGKGLEWVAYISSGSYTIYSADSVKGRFTISRDNAKNSLYLQMNSLRAEDTAVYYCARRAPNSFYEYYFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEACVLGGGGSGGGGSQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGGGGSGGGGSQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 39 - protein, 20 aa, Artificial Sequence
  Description of Artificial Sequence Synthetic peptide
GGGGSGGGGSGGGGSGGGGS

SEQ ID NO: 40 - protein, 15 aa, Artificial Sequence
  Description of Artificial Sequence Synthetic peptide
GGGGSGGGGSGGGGS

SEQ ID NO: 41 - protein, 10 aa, Artificial Sequence
  Description of Artificial Sequence Synthetic peptide
GGGGSGGGGS

SEQ ID NO: 42 - protein, 9 aa, Artificial Sequence
  Description of Artificial Sequence Synthetic peptide
GGGGSGGGS

SEQ ID NO: 43 - protein, 5 aa, Artificial Sequence
  Description of Artificial Sequence Synthetic peptide
GGGGS